(19) United States
(12) Patent Application Publication
(10) Pub. No.: US 2025/0059295 A1
(43) Pub. Date: Feb. 20, 2025

(54) STEAP2 DIRECTED T-CELL ENGAGERS AND COMPOSITIONS THEREOF

(71) Applicant: MedImmune, LLC, Gaithersburg, MD (US)

(72) Inventors: Zachary BRITTON, Gaithersburg, MD (US); Sao Fong CHEUNG, Gaithersburg, MD (US); Jeong Min HAN, Gaithersburg, MD (US); Yariv MAZOR, Gaithersburg, MD (US); Suzanne SITNIKOVA, Cambridge (GB); Dewald van DYK, Gaithersburg, MD (US); Even WALSENG, Gaithersburg, MD (US); Chunning YANG, Gaithersburg, MD (US); Simon DOVEDI, Cambridge (GB); Saso CEMERSKI, Gaithersburg, MD (US); Natalie BURROWS, Cambridge (GB); Nikolaos IOANNOU, Cambridge (GB); Oisin HUHN, Cambridge (GB)

(21) Appl. No.: 18/631,627

(22) Filed: Apr. 10, 2024

Related U.S. Application Data
(60) Provisional application No. 63/495,547, filed on Apr. 11, 2023.

(51) Int. Cl. C07K 16/30; A61K 39/00; C07K 16/28

(52) U.S. Cl. CPC C07K 16/3069; C07K 16/2809; A61K 2039/505; C07K 2317/31; C07K 2317/522; C07K 2317/524; C07K 2317/526; C07K 2317/565

(57) ABSTRACT

Provided are T ell engaging molecules that bind an antigen on a non-immune cell and an antigen on an immune cell. The T cell engaging molecules can bind STEAP2 on a cancer cell and, e.g., a CD3 on a T cell. The T cell engaging molecules can also bind STEAP2 on a cancer cell and, e.g., a CD8 on a T cell. Alternatively, the T cell engaging molecules can bind STEAP2 on a cancer cell and both CD3 and CD8 on a T cell.

### CROSS-REFERENCE TO RELATED APPLICATIONS

This application claims the benefit of U.S. Provisional Patent Application No. 63/495,547, filed Apr. 11, 2023, which is incorporated herein by reference in its entirety.

### REFERENCE TO SEQUENCE LISTING SUBMITTED ELECTRONICALLY

The content of the electronically submitted sequence listing (Name: STEAP2TED-100-US-NP_ST26.xml; Size: 182,494 bytes; and Date of Creation: Apr. 10, 2024), filed with the application, is incorporated herein by reference in its entirety.

### BACKGROUND

Multivalent binding molecules, which recognize two or more different epitopes, are of interest in diagnostic and therapeutic approaches. However, their generation presents challenges. Promiscuous pairing of heavy and light chains expressed in one cell can result in the production of several different molecules, with only one pairing being the desired one and the remaining pairings resulting in non-functional or monospecific molecules.

Various strategies have been developed in an attempt to overcome this problem and encourage the correct assembly of the desired multivalent molecule. However, with the introduction of each further binding arm the possibility for mispairings increases.

Therefore, there is a need for additional mechanisms to improve pairing of polypeptide chains in multivalent molecules and facilitate their efficient production.

### BRIEF SUMMARY

Provided is a T cell engaging molecule comprising: (a) an antigen binding arm that binds an epitope on human six transmembrane epithelial antigen of prostate-2 (STEAP2) and comprises a heavy chain comprising a heavy chain variable domain (VH) comprising a variable heavy chain complementarity determining region 1 (VH-CDR1) selected from SEQ ID NOs: 1, 9, 17, 103, 111, 127, 135, and 143; a VH-CDR2 selected from SEQ ID NOs: 2, 10, 18, 104, 112, 128, 136, and 144; a VH-CDR3 selected from SEQ ID NOs: 3, 11, 19, 94, 96, 98, 105, 113, 129, 137, and 145; a heavy chain CH1 domain; and a light chain comprising a light chain variable domain (VL) comprising a variable light chain complementarity determining region 1 (VL-CDR1) selected from SEQ ID NOs: 4, 12, 20, 100, 108, 130, 138, and 146; a VL-CDR2 selected from SEQ ID NOs: 5, 13, 21, 101, 109, 131, 139, and 147; a VL-CDR3 selected from SEQ ID NOs: 6, 14, 22, 102, 110, 132, 140, and 148; and a light chain constant domain; (b) a first T cell binding arm comprising a heavy chain comprising a heavy chain variable domain (VH) comprising a VH-CDR1 selected from SEQ ID NOs: 36, 40, and 44; a VH-CDR2 selected from SEQ ID NOs: 37, 41, and 45; a VH-CDR3 selected from SEQ ID NOs: 38, 42, and 46; and a heavy chain CH1 domain; and a light chain comprising a light chain variable domain (VL) comprising a VL-CDR1 selected from SEQ ID NOs: 27 and 31; a VL-CDR2 selected from SEQ ID NOs: 28 and 32; a VL-CDR3 selected from SEQ ID NOs: 29 and 33; and a light chain constant domain; and (c) an Fc domain comprising a first Fc region and a second Fc region, each Fc region comprising a CH2 domain and a CH3 domain; said Fc domain further comprising at least one modification to promote heterodimerization.

In some aspects, provided is a trivalent T cell engaging molecule comprising the T cell engaging molecule as described and further comprising (d) a second T cell binding arm that binds to cluster of differentiation 8 (CD8) and comprises a heavy chain comprising a heavy chain variable domain (VH) comprising a VH-CDR1 set forth in SEQ ID NO: 48, a VH-CDR2 set forth in SEQ ID NO: 49, and a VH-CDR3 set forth in SEQ ID NO: 50, and a heavy chain CH1 domain; and a light chain comprising a light chain variable domain (VL) comprising a VL-CDR1 set forth in SEQ ID NO: 51, a VL-CDR2 set forth in SEQ ID NO: 52, and a VL-CDR3 set forth in SEQ ID NO: 53, and a light chain constant domain.

In some aspects, the heavy chain of the second T cell binding arm is attached to the heavy chain of the first T cell binding arm through a linker.

In some aspects, the heavy chain of the second T cell binding arm is attached to the heavy chain of the antigen binding arm through a linker.

In some aspects, the linker comprises the amino acid sequence of SEQ ID NO: 89.

In some aspects, the linker comprises from 1 to about 10 copies of SEQ ID NO: 89.

In some aspects, the linker comprises 2 copies of SEQ ID NO: 89.

In some aspects, one of the two CH3 domains of the T cell engaging molecule or trivalent T cell engaging molecule comprises a knob mutation, and the other of the two CH3 domain comprises a hole mutation.

In some aspects, the CH1 domain and the light chain constant domain of one or more of each of the (a) antigen binding arm, (b) the first T cell binding arm, and (d) the second T cell binding arm further comprises a charge pair substitution comprising a first charged amino acid substitution in the CH1 domain and a second charged amino acid substitution in the light chain constant domain, wherein the first charged amino acid substitution and the second charged amino acid substitution have an opposite charge.

In some aspects, the light chain constant domain of one or more of each of (a), (b), and (d) of the T cell engaging molecule or trivalent T cell engaging molecule is a lambda light chain constant domain (CLλ) and wherein the charge pair is a lambda charge pair comprising a positively charged amino acid residue selected from arginine, lysine or histidine, and a negatively charged amino acid residue selected from aspartic acid, glutamic acid, serine or threonine, wherein the numbering is according to the EU index and wherein the charged amino acids of the lambda charge pair are located at one or more of the following positions:
(i) position 117 in the CLλ and position 141 in the CH1 domain;
(ii) position 117 in the CLλ and position 185 in the CH1 domain;
(iii) position 119 in the CLλ and position 128 in the CH1 domain;
(iv) position 134 in the CLλ and position 128 in the CH1 domain;
(v) position 134 in the CLλ and position 145 in the CH1 domain;
(vi) position 134 in the CLλ and position 183 in the CH1 domain;
(vii) position 136 in the CLλ and position 185 in the CH1 domain;
(viii) position 178 in the CLλ and position 173 in the CH1 domain; and/or
(ix) position 117 in the CLλ and position 187 in the CH1 domain.

In some aspects, the charged amino acids of the lambda charge pair of the T cell engaging molecule or trivalent T cell engaging molecule are as follows:
(i) the charged amino acid at position 117 is arginine and the charged amino acid at position 141 is aspartic acid; the charged amino acid at position 117 is arginine and the charged amino acid at position 141 is glutamic acid; the charged amino acid at position 117 is arginine and the charged amino acid at position 141 is serine; the charged amino acid at position 117 is arginine and the charged amino acid at position 141 is threonine; the charged amino acid at position 117 is lysine and the charged amino acid at position 141 is aspartic acid; the charged amino acid at position 117 is lysine and the charged amino acid at position 141 is glutamic acid; the charged amino acid at position 117 is lysine and the charged amino acid at position 141 is serine; or the charged amino acid at position 117 is lysine and the charged amino acid at position 141 is threonine;
(ii) the charged amino acid at position 117 is arginine and the charged amino acid at position 185 is aspartic acid; the charged amino acid at position 117 is arginine and the charged amino acid at position 185 is glutamic acid; the charged amino acid at position 117 is arginine and the charged amino acid at position 185 is serine; the charged amino acid at position 117 is arginine and the charged amino acid at position 185 is threonine; the charged amino acid at position 117 is lysine and the charged amino acid at position 185 is aspartic acid; the charged amino acid at position 117 is lysine and the charged amino acid at position 185 is glutamic acid; the charged amino acid at position 117 is lysine and the charged amino acid at position 185 is serine; or the charged amino acid at position 117 is lysine and the charged amino acid at position 185 is threonine;
(iii) the charged amino acid at position 119 is arginine and the charged amino acid at position 128 is aspartic acid; the charged amino acid at position 119 is arginine and the charged amino acid at position 128 is glutamic acid; the charged amino acid at position 119 is arginine and the charged amino acid at position 128 is serine; the charged amino acid at position 119 is arginine and the charged amino acid at position 128 is threonine; the charged amino acid at position 119 is lysine and the charged amino acid at position 128 is aspartic acid; the charged amino acid at position 119 is lysine and the charged amino acid at position 128 is glutamic acid; the charged amino acid at position 119 is lysine and the charged amino acid at position 128 is serine; or the charged amino acid at position 119 is lysine and the charged amino acid at position 128 is threonine;
(iv) the charged amino acid at position 134 is arginine and the charged amino acid at position 128 is aspartic acid; the charged amino acid at position 134 is arginine and the charged amino acid at position 128 is glutamic acid; the charged amino acid at position 134 is arginine and the charged amino acid at position 128 is serine; the charged amino acid at position 134 is arginine and the charged amino acid at position 128 is threonine; the charged amino acid at position 134 is lysine and the charged amino acid at position 128 is aspartic acid; the charged amino acid at position 134 is lysine and the charged amino acid at position 128 is glutamic acid; the charged amino acid at position 134 is lysine and the charged amino acid at position 128 is serine; or the charged amino acid at position 134 is lysine and the charged amino acid at position 128 is threonine;
(v) the charged amino acid at position 134 is arginine and the charged amino acid at position 145 is aspartic acid; the charged amino acid at position 134 is arginine and the charged amino acid at position 145 is glutamic acid; the charged amino acid at position 134 is arginine and the charged amino acid at position 145 is serine; the charged amino acid at position 134 is arginine and the charged amino acid at position 145 is threonine; the charged amino acid at position 134 is lysine and the charged amino acid at position 145 is aspartic acid; the charged amino acid at position 134 is lysine and the charged amino acid at position 145 is glutamic acid; the charged amino acid at position 134 is lysine and the charged amino acid at position 145 is serine; or the charged amino acid at position 134 is lysine and the charged amino acid at position 145 is threonine;
(vi) the charged amino acid at position 134 is arginine and the charged amino acid at position 183 is aspartic acid; the charged amino acid at position 134 is arginine and the charged amino acid at position 183 is glutamic acid; the charged amino acid at position 134 is arginine and the charged amino acid at position 183 is serine; the charged amino acid at position 134 is arginine and the charged amino acid at position 183 is threonine; the charged amino acid at position 134 is lysine and the charged amino acid at position 183 is aspartic acid; the charged amino acid at position 134 is lysine and the charged amino acid at position 183 is glutamic acid; the charged amino acid at position 134 is lysine and the charged amino acid at position 183 is serine; or the charged amino acid at position 134 is lysine and the charged amino acid at position 183 is threonine;
(vii) the charged amino acid at position 136 is arginine and the charged amino acid at position 185 is aspartic acid; the charged amino acid at position 136 is arginine and the charged amino acid at position 185 is glutamic acid; the charged amino acid at position 136 is arginine and the charged amino acid at position 185 is serine; the charged amino acid at position 136 is arginine and the charged amino acid at position 185 is threonine; the charged amino acid at position 136 is lysine and the charged amino acid at position 185 is aspartic acid; the charged amino acid at position 136 is lysine and the charged amino acid at position 185 is glutamic acid; the charged amino acid at position 136 is lysine and the charged amino acid at position 185 is serine; or the charged amino acid at position 136 is lysine and the charged amino acid at position 185 is threonine;
(viii) the charged amino acid at position 178 is arginine and the charged amino acid at position 173 is aspartic acid; the charged amino acid at position 178 is arginine and the charged amino acid at position 173 is glutamic acid; the charged amino acid at position 178 is arginine and the charged amino acid at position 173 is serine; the charged amino acid at position 178 is arginine and the charged amino acid at position 173 is threonine; the charged amino acid at position 178 is lysine and the charged amino acid at position 173 is aspartic acid; the charged amino acid at position 178 is lysine and the charged amino acid at position 173 is glutamic acid; the charged amino acid at position 178 is lysine and the charged amino acid at position 173 is serine; or the charged amino acid at position 178 is lysine and the charged amino acid at position 173 is threonine; and/or
(ix) the charged amino acid at position 117 is arginine and the charged amino acid at position 187 is aspartic acid; the charged amino acid at position 117 is arginine and the charged amino acid at position 187 is glutamic acid; the charged amino acid at position 117 is arginine and the charged amino acid at position 187 is serine; the charged amino acid at position 117 is arginine and the charged amino acid at position 187 is threonine; the charged amino acid at position 117 is lysine and the charged amino acid at position 187 is aspartic acid; the charged amino acid at position 117 is lysine and the charged amino acid at position 187 is glutamic acid; the charged amino acid at position 117 is lysine and the charged amino acid at position 187 is serine; or the charged amino acid at position 117 is lysine and the charged amino acid at position 187 is threonine.

In some aspects, the light chain constant domain of one or more of each of (a), (b), and (d) is a kappa light chain constant domain (CLκ) and wherein the charge pair is a kappa charge pair comprising a positively charged amino acid residue selected from arginine, lysine or histidine, and a negatively charged amino acid residue selected from aspartic acid, glutamic acid, serine or threonine, wherein the numbering is according to the EU index and wherein the kappa charge pair is located at position 133 in the CLκ and position 183 in the CH1 domain.

In some aspects, the charged amino acids of the kappa charge pair of the T cell engaging molecule or trivalent T cell engaging molecule are as follows: (i) the charged amino acid at position 133 is glutamic acid and the charged amino acid at position 183 is lysine; or (ii) the charged amino acid at position 133 is lysine and the charged amino acid at position 183 is glutamic acid.

In some aspects, the light chain constant domain of one of (a), (b), and (d) is a CLλ and wherein the charge pair is a lambda charge pair and the light chain constant domain of a second and a third of (a), (b), and (d) is a CLλ or a CLκ and wherein the charge pair is a lambda or a kappa charge pair and the second of (a), (b), and (d) comprises a charged amino acid in the CH1 domain of a same charge as a charged amino acid in the CH1 domain of the third of (a), (b), and (d).

In some aspects, the light chain constant domain of one of (a), (b), and (d) is a CLλ and wherein the charge pair is a lambda charge pair and the light chain constant domain of a second and a third of (a), (b), and (d) is a CLκ and wherein the charge pair is a kappa charge pair and the second of (a), (b), and (d) comprises a charged amino acid in the CH1 domain of a same charge as a charged amino acid in the CH1 domain of the third of (a), (b), and (d).

In some aspects, the CH1 domain of (a), (b), and/or (d) of the T cell engaging molecule or trivalent T cell engaging molecule is capable of being linked to the light chain constant domain through an engineered disulfide link.

In some aspects, the CH1 domain of (b) and (d) of the T cell engaging molecule or trivalent T cell engaging molecule is capable of being linked to the light chain constant domain through an engineered disulfide link and the CH1 domain of (a) is linked to the light chain constant domain through a native disulfide link.

In some aspects, the CH1 domain of the T cell engaging molecule or trivalent T cell engaging molecule that is capable of being linked to the light chain constant domain through an engineered disulfide link comprises:
(i) a substitution of a native cysteine to a non-cysteine amino acid,
(ii) a substitution of a native non-cysteine amino acid to a cysteine;
   and the light chain constant domain comprises
(i) a substitution of a native cysteine to a non-cysteine amino acid,
(ii) a substitution of a native non-cysteine amino acid to a cysteine; and
   wherein the substituted cysteine of the light chain constant domain and the substituted cysteine of the CH1 domain can form a disulfide bond.

In some aspects, the CH1 domain of the T cell engaging molecule or trivalent T cell engaging molecule that is capable of being linked to the light chain constant domain through an engineered disulfide link comprises:
(i) a substitution of a native cysteine to a non-cysteine amino acid at position 220,
(ii) a substitution of a native non-cysteine amino acid to a cysteine at position 126;
   and the light chain constant domain comprises:
(i) a substitution of a native cysteine to a non-cysteine amino acid at position 212,
(ii) a substitution of a native non-cysteine amino acid to a cysteine at position 122,
   wherein the cysteine at position 126 of the CH1 domain and the cysteine at position 122 of the light chain constant domain can form a disulfide bond; wherein the numbering is according to the EU index.

In some aspects, the CH1 domain of the T cell engaging molecule or trivalent T cell engaging molecule that is capable of being linked to the light chain constant domain through an engineered disulfide link comprises:
(i) a substitution of a native cysteine to a non-cysteine amino acid at position 220,
(ii) a substitution of a native non-cysteine amino acid to a cysteine at position 126;
   and the light chain constant domain comprises:
(i) a substitution of a native cysteine to a non-cysteine amino acid at position 214,
(ii) a substitution of a native non-cysteine amino acid to a cysteine at position 121,
   wherein the cysteine at position 126 of the CH1 domain and the cysteine at position 121 of the light chain constant domain can form a disulfide bond; wherein the numbering is according to the EU index.

In some aspects, the antigen binding arm of the T cell engaging molecule or trivalent T cell engaging molecule comprises a VH comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 15, 23, 93, 95, 97, 106, 114, 133, 141, and 149.

In some aspects, the antigen binding arm of the T cell engaging molecule or trivalent T cell engaging molecule is attached to the Fc domain and comprises a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 66, 67, 69, 72, 73, and 76.

In some aspects, the antigen binding arm of the T cell engaging molecule or trivalent T cell engaging molecule comprises a VL comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 16, 24, 107, 115, 134, 142, and 150.

In some aspects, the antigen binding arm of the T cell engaging molecule or trivalent T cell engaging molecule comprises a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 63.

In some aspects, the VL of the T cell binding arm of the T cell engaging molecule or the VL of the first or second T cell binding arm of the trivalent T cell engaging molecule comprises a VL-CDR1 selected from SEQ ID NOs: 27 and 31; a VL-CDR2 selected from SEQ ID NOs: 28 and 32; and a VL-CDR3 selected from SEQ ID NOs: 29 and 33; and the VH comprises a VH-CDR1 selected from SEQ ID NOs: 36, 40, and 44; a VH-CDR2 selected from SEQ ID NOs: 37, 41 and 45; and a VH-CDR3 selected from SEQ ID NOs: 38, 42, and 46.

In some aspects, the VL of the T cell binding arm of the T cell engaging molecule or the VL of the first or second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30 or 34.

In some aspects, the light chain constant domain of the T cell engaging molecule or the light chain constant domain of the first or second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 60 or 61.

In some aspects, the VH of the T cell binding arm of the T cell engaging molecule or the VH of the first or second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NOs: 39, 43, or 47.

In some aspects, the heavy chain of the T cell binding arm of the T cell engaging molecule or the first or second T cell binding arm of the trivalent T cell engaging molecule is attached to the Fc domain and comprises a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 74, 75, 77, 78, and 79.

In some aspects, the VH of the second T cell binding arm of the trivalent T cell engaging molecule comprises a VH-CDR1 set forth in SEQ ID NO: 48; a VH-CDR2 set forth in SEQ ID NO: 49; a VH-CDR3 set forth in SEQ ID NO: 50; and the VL comprises a VL-CDR1 set forth in SEQ ID NO: 51; a VL-CDR2 set forth in SEQ ID NO: 52; and a VL-CDR3 set forth in SEQ ID NO: 53.

In some aspects, the VL of the second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54.

In some aspects, the constant light chain domain of the second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 64.

In some aspects, the VH of the second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56.

In some aspects, the CH1 domain of the second T cell binding arm of the trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 57, 66, 67, 69, 74, 75, 77, 78, and 79.

In some aspects, the antigen binding arm of the T cell engaging molecule or trivalent T cell engaging molecule binds an epitope on an extracellular loop of STEAP2.

In some aspects, the T cell engaging molecule comprises SEQ ID NOs: 25, 26, 35, 81, and 92. In some aspects, the trivalent T cell engaging molecule comprises SEQ ID NOs: 25, 26, 35, 55 and 58.

In some aspects, provided is a trivalent T cell engaging molecule comprising: (a) a first antigen binding arm and a second antigen binding arm that each bind an epitope on human six transmembrane epithelial antigen of prostate-2 (STEAP2) and each comprise a heavy chain comprising a heavy chain variable domain (VH) comprising a variable heavy chain complementarity determining region 1 (VH-CDR1) selected from SEQ ID NOs: 1, 9, 17, 103, 111, 127, 135, and 143; a VH-CDR2 selected from SEQ ID NOs: 2, 10, 18, 104, 112, 128, 136, and 144; a VH-CDR3 selected from SEQ ID NOs: 3, 11, 19, 94, 96, 98, 105, 113, 129, 137, and 145; a heavy chain CH1 domain; and a light chain comprising a light chain variable domain (VL) comprising a variable light chain complementarity determining region 1 (VL-CDR1) selected from SEQ ID NOs: 4, 12, 20, 100, 108, 130, 138, and 146; a VL-CDR2 selected from SEQ ID NOs: 5, 13, and 21; a VL-CDR3 selected from SEQ ID NOs: 6, 14, 22, 102, 110, 132, 140, and 148; and a light chain constant domain; (b) a first T cell binding arm that binds to cluster of differentiation 3 (CD3) and comprises a heavy chain comprising a VH comprising a VH-CDR1 selected from SEQ ID NOs: 36, 40, and 44; a VH-CDR2 selected from SEQ ID NOs: 37, 41, and 45; a VH-CDR3 selected from SEQ ID NOs: 38, 42, and 46; and a heavy chain CH1 domain; and a light chain comprising a VL comprising a VL-CDR1 selected from SEQ ID NOs: 27 and 31; a VL-CDR2 selected from SEQ ID NOs: 28 and 32; a VL-CDR3 selected from SEQ ID NOs: 29 and 33; and a light chain constant domain; and (c) an Fc domain comprising a first Fc region and a second Fc region, each Fc region comprising a CH2 domain and a CH3 domain, and further comprising at least one modification to promote heterodimerization; and wherein the heavy chain of the first antigen binding arm and the heavy chain of the first T cell binding arm are attached to the Fc domain, and the heavy chain of the second antigen binding arm is attached to the heavy chain of the first T cell binding arm.

In some aspects, provided is a tetravalent T cell engaging molecule comprising the trivalent T cell engaging molecule described above and further comprising (d) a second T cell binding arm that binds to cluster of differentiation 8 (CD8) and comprises a heavy chain comprising a variable heavy domain (VHH) comprising a VH-CDR1 set forth in SEQ ID NO: 84; a VH-CDR2 set forth in SEQ ID NO: 85; and a VH-CDR3 set forth in SEQ ID NO: 86.

In some aspects, the heavy chain of the second T cell binding arm of the tetravalent T cell engaging molecule is attached to the heavy chain of the first antigen binding arm.

In some aspects, the heavy chain of the second antigen binding arm of the trivalent or tetravalent T cell engaging molecule is attached to the heavy chain of the first T cell binding arm through a linker.

In some aspects, the heavy chain of the second T cell binding arm of the tetravalent T cell engaging molecule is attached to the heavy chain of the first antigen binding arm through a linker.

In some aspects, the linker comprises the amino acid sequence of SEQ ID NO: 89.

In some aspects, the linker comprises from 1 to about 10 copies of SEQ ID NO: 89.

In some aspects, the linker comprises 2 copies of SEQ ID NO: 89.

In some aspects, one of the two CH3 domains of the trivalent or tetravalent T cell engaging molecule comprises a knob mutation, and the other of the two CH3 domain comprises a hole mutation.

In some aspects, the CH1 domain and the light chain constant domain of one or more of each of the (a) first antigen binding arm or second antigen binding arm, and (b) the first T cell binding arm, comprises a charge pair substitution comprising a first charged amino acid substitution in the CH1 domain and a second charged amino acid substitution in the light chain constant domain, wherein the first charged amino acid substitution and the second charged amino acid substitution have an opposite charge.

In some aspects, the light chain constant domain of one or more of each of (a) the first antigen binding arm or the second antigen binding arm, and (b) the first T cell binding arm is a lambda light chain constant domain (CLλ) and the charge pair is a lambda charge pair comprising a positively charged amino acid residue selected from arginine, lysine or histidine, and a negatively charged amino acid residue selected from aspartic acid, glutamic acid, serine or threonine, wherein the numbering is according to the EU index and wherein the lambda charge pair is located at one or more of the following positions:
(i) position 117 in the CLλ and position 141 in the CH1 domain;
(ii) position 117 in the CLλ and position 185 in the CH1 domain;
(iii) position 119 in the CLλ and position 128 in the CH1 domain;
(iv) position 134 in the CLλ and position 128 in the CH1 domain;
(v) position 134 in the CLλ and position 145 in the CH1 domain;
(vi) position 134 in the CLλ and position 183 in the CH1 domain;
(vii) position 136 in the CLλ and position 185 in the CH1 domain;
(viii) position 178 in the CLλ and position 173 in the CH1 domain; and
(ix) position 117 in the CLλ and position 187 in the CH1 domain.

In some aspects, the charged amino acids of the lambda charge pair of the trivalent or tetravalent T cell engaging molecule are as follows:
(i) the charged amino acid at position 117 is arginine and the charged amino acid at position 141 is aspartic acid; the charged amino acid at position 117 is arginine and the charged amino acid at position 141 is glutamic acid; the charged amino acid at position 117 is arginine and the charged amino acid at position 141 is serine; the charged amino acid at position 117 is arginine and the charged amino acid at position 141 is threonine; the charged amino acid at position 117 is lysine and the charged amino acid at position 141 is aspartic acid; the charged amino acid at position 117 is lysine and the charged amino acid at position 141 is glutamic acid; the charged amino acid at position 117 is lysine and the charged amino acid at position 141 is serine; or the charged amino acid at position 117 is lysine and the charged amino acid at position 141 is threonine;
(ii) the charged amino acid at position 117 is arginine and the charged amino acid at position 185 is aspartic acid; the charged amino acid at position 117 is arginine and the charged amino acid at position 185 is glutamic acid; the charged amino acid at position 117 is arginine and the charged amino acid at position 185 is serine; the charged amino acid at position 117 is arginine and the charged amino acid at position 185 is threonine; the charged amino acid at position 117 is lysine and the charged amino acid at position 185 is aspartic acid; the charged amino acid at position 117 is lysine and the charged amino acid at position 185 is glutamic acid; the charged amino acid at position 117 is lysine and the charged amino acid at position 185 is serine; or the charged amino acid at position 117 is lysine and the charged amino acid at position 185 is threonine;
(iii) the charged amino acid at position 119 is arginine and the charged amino acid at position 128 is aspartic acid; the charged amino acid at position 119 is arginine and the charged amino acid at position 128 is glutamic acid; the charged amino acid at position 119 is arginine and the charged amino acid at position 128 is serine; the charged amino acid at position 119 is arginine and the charged amino acid at position 128 is threonine; the charged amino acid at position 119 is lysine and the charged amino acid at position 128 is aspartic acid; the charged amino acid at position 119 is lysine and the charged amino acid at position 128 is glutamic acid; the charged amino acid at position 119 is lysine and the charged amino acid at position 128 is serine; or the charged amino acid at position 119 is lysine and the charged amino acid at position 128 is threonine;
(iv) the charged amino acid at position 134 is arginine and the charged amino acid at position 128 is aspartic acid; the charged amino acid at position 134 is arginine and the charged amino acid at position 128 is glutamic acid; the charged amino acid at position 134 is arginine and the charged amino acid at position 128 is serine; the charged amino acid at position 134 is arginine and the charged amino acid at position 128 is threonine; the charged amino acid at position 134 is lysine and the charged amino acid at position 128 is aspartic acid; the charged amino acid at position 134 is lysine and the charged amino acid at position 128 is glutamic acid; the charged amino acid at position 134 is lysine and the charged amino acid at position 128 is serine; or the charged amino acid at position 134 is lysine and the charged amino acid at position 128 is threonine;
(v) the charged amino acid at position 134 is arginine and the charged amino acid at position 145 is aspartic acid; the charged amino acid at position 134 is arginine and the charged amino acid at position 145 is glutamic acid; the charged amino acid at position 134 is arginine and the charged amino acid at position 145 is serine; the charged amino acid at position 134 is arginine and the charged amino acid at position 145 is threonine; the charged amino acid at position 134 is lysine and the charged amino acid at position 145 is aspartic acid; the charged amino acid at position 134 is lysine and the charged amino acid at position 145 is glutamic acid; the charged amino acid at position 134 is lysine and the charged amino acid at position 145 is serine; or the charged amino acid at position 134 is lysine and the charged amino acid at position 145 is threonine;
(vi) the charged amino acid at position 134 is arginine and the charged amino acid at position 183 is aspartic acid; the charged amino acid at position 134 is arginine and the charged amino acid at position 183 is glutamic acid; the charged amino acid at position 134 is arginine and the charged amino acid at position 183 is serine; the charged amino acid at position 134 is arginine and the charged amino acid at position 183 is threonine; the charged amino acid at position 134 is lysine and the charged amino acid at position 183 is aspartic acid; the charged amino acid at position 134 is lysine and the charged amino acid at position 183 is glutamic acid; the charged amino acid at position 134 is lysine and the charged amino acid at position 183 is serine; or the charged amino acid at position 134 is lysine and the charged amino acid at position 183 is threonine;
(vii) the charged amino acid at position 136 is arginine and the charged amino acid at position 185 is aspartic acid; the charged amino acid at position 136 is arginine and the charged amino acid at position 185 is glutamic acid; the charged amino acid at position 136 is arginine and the charged amino acid at position 185 is serine; the charged amino acid at position 136 is arginine and the charged amino acid at position 185 is threonine; the charged amino acid at position 136 is lysine and the charged amino acid at position 185 is aspartic acid; the charged amino acid at position 136 is lysine and the charged amino acid at position 185 is glutamic acid; the charged amino acid at position 136 is lysine and the charged amino acid at position 185 is serine; or the charged amino acid at position 136 is lysine and the charged amino acid at position 185 is threonine;
(viii) the charged amino acid at position 178 is arginine and the charged amino acid at position 173 is aspartic acid; the charged amino acid at position 178 is arginine and the charged amino acid at position 173 is glutamic acid; the charged amino acid at position 178 is arginine and the charged amino acid at position 173 is serine; the charged amino acid at position 178 is arginine and the charged amino acid at position 173 is threonine; the charged amino acid at position 178 is lysine and the charged amino acid at position 173 is aspartic acid; the charged amino acid at position 178 is lysine and the charged amino acid at position 173 is glutamic acid; the charged amino acid at position 178 is lysine and the charged amino acid at position 173 is serine; or the charged amino acid at position 178 is lysine and the charged amino acid at position 173 is threonine; and/or
(ix) the charged amino acid at position 117 is arginine and the charged amino acid at position 187 is aspartic acid; the charged amino acid at position 117 is arginine and the charged amino acid at position 187 is glutamic acid; the charged amino acid at position 117 is arginine and the charged amino acid at position 187 is serine; the charged amino acid at position 117 is arginine and the charged amino acid at position 187 is threonine; the charged amino acid at position 117 is lysine and the charged amino acid at position 187 is aspartic acid; the charged amino acid at position 117 is lysine and the charged amino acid at position 187 is glutamic acid; the charged amino acid at position 117 is lysine and the charged amino acid at position 187 is serine; or the charged amino acid at position 117 is lysine and the charged amino acid at position 187 is threonine.

In some aspects, the light chain constant domain of one or more of each of (a) the first antigen binding arm or the second antigen binding arm, and (b) the first T cell binding arm is a kappa light chain constant domain (CLκ) and the charge pair is a kappa charge pair comprising a positively charged amino acid residue selected from arginine, lysine or histidine, and a negatively charged amino acid residue selected from aspartic acid, glutamic acid, serine or threonine, wherein the numbering is according to the EU index and wherein the kappa charge pair is located at position 133 in the CLκ and position 183 in the CH1.

In some aspects, the charged amino acids of the kappa charge pair of the trivalent or tetravalent T cell engaging molecule are as follows: (i) the charged amino acid at position 133 is glutamic acid and the charged amino acid at position 183 is lysine; or (ii) the charged amino acid at position 133 is lysine and the charge amino acid at position 183 is glutamic acid.

In some aspects, the light chain constant domain of one of (a), (b), and (d) is a CLλ and wherein the charge pair is a lambda charge pair and the light chain constant domain of a second and a third of (a), (b), and (d) is a CLλ or CLκ and wherein the charge pair is a lambda or a kappa charge pair and the second of (a), (b), and (d) comprises a charged amino acid in the CH1 domain of a same charge as a charged amino acid in the CH1 domain of the third of (a), (b), and (d).

In some aspects, the light chain constant domain of one of (a), (b), and (d) is a CLλ and wherein the charge pair is a lambda charge pair and the light chain constant domain of a second and a third of (a), (b), and (d) is a CLκ and wherein the charge pair is a kappa charge pair and the second of (a), (b), and (d) comprises a charged amino acid in the CH1 domain of a same charge as a charged amino acid in the CH1 domain of the third of (a), (b), and (d).

In some aspects, the CH1 domain of (a) and/or (b) of the trivalent T cell engaging molecule or the CH1 domain of (a), (b), and/or (d) of the tetravalent T cell engaging molecules is capable of forming a disulfide link to the light chain constant domain of (a) and/or (b) of the trivalent T cell engaging molecule or the light chain of (a), (b), and/or (d) of the tetravalent T cell engaging molecule through an engineered disulfide link.

In some aspects, the CH1 domain of (b) of the trivalent or tetravalent T cell engaging molecule is capable of forming a disulfide link to the light chain constant domain through an engineered disulfide link and the CH1 domain of (a) is linked to the light chain constant domain through a native disulfide link.

In some aspects, CH1 domain of (b) and (d) of the tetravalent T cell engaging molecule are capable of forming a disulfide link to the light chain constant domain of (b) and (d) through an engineered disulfide link and the CH1 domain of (a) is linked to the light chain constant domain of (a) through a native disulfide link.

In some aspects, the CH1 domain of the first T cell binding arm of the trivalent or tetravalent T cell engaging molecule comprises
(i) a substitution of a native cysteine to a non-cysteine amino acid,
(ii) a substitution of a native non-cysteine amino acid to a cysteine; and
   the light chain constant domain of the second T cell binding arm comprises:
(i) a substitution of a native cysteine to a non-cysteine amino acid,
(ii) a substitution of a native non-cysteine amino acid to a cysteine;
   wherein the substituted cysteine of the CH1 domain of the first T cell binding arm and the substituted cysteine of the light chain constant domain of the first T cell binding arm can form a disulfide bond.

In some aspects, the CH1 domain of the first T cell binding arm of the trivalent or tetravalent T cell engaging molecule comprises:
(i) a substitution of a native cysteine to a non-cysteine amino acid at position 220,
(ii) a substitution of a native non-cysteine amino acid to a cysteine at position 126;
   and the light chain constant domain comprises:
(i) a substitution of a native cysteine to a non-cysteine amino acid at position 212,
(ii) a substitution of a native non-cysteine amino acid to a cysteine at position 122,
   wherein the cysteine at position 126 of the CH1 domain and the cysteine at position 122 of the light chain constant domain can form a disulfide bond; wherein the numbering is according to the EU index.

In some aspects, the light chains of the first and second antigen binding arms of the trivalent or tetravalent T cell engaging molecule comprise a native cysteine and the CH1 domain of the first and second antigen binding arms comprise a native cysteine, wherein the native cysteine of the CH1 domain of the first antigen binding arm and the native cysteine of the light chain constant domain of the first antigen binding arm can form a disulfide bond and the native cysteine of the CH1 of the second antigen binding arm and the native cysteine of the light chain constant domain of the second antigen binding arm can form a disulfide bond.

In some aspects, the VH of the first antigen binding arm of the trivalent or tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 7, 15, 23, 95, 97, 99, 106, 114, 133, 141, and 149.

In some aspects, the CH1 of the first antigen binding arm of the trivalent or tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 67 and 93.

In some aspects, the VL of the first antigen binding arm of the trivalent or tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 8, 16, 24, 107, 115, 134, 142, and 150.

In some aspects, the light chain constant domain of the first antigen binding arm of the trivalent or tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 63.

In some aspects, the VL of the first T cell binding arm of the trivalent or tetravalent T cell engaging molecule comprises a VL-CDR1 set forth in SEQ ID NO: 27 or 31; a VL-CDR2 set forth in SEQ ID NO: 28 or 32; a VL-CDR3 set forth in SEQ ID NO: 29 or 33; and the VH of the first T cell binding arm comprises a VH-CDR1 set forth in SEQ ID NO: 36, 40, or 44; a VH-CDR2 set forth in SEQ ID NO: 37, 41 or 45; and a VH-CDR3 set forth in SEQ ID NO: 38, 42, or 46.

In some aspects, the VL of the first T cell binding arm of the trivalent or tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30 or 34.

In some aspects, the VH of the first T cell binding arm of the trivalent or tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NOs: 39, 43, and 47.

In some aspects, the VH of the second T cell binding arm of the tetravalent T cell engaging molecule comprises a VH-CDR1 set forth in SEQ ID NO: 84; a VH-CDR2 set forth in SEQ ID NO: 85; and a VH-CDR3 set forth in SEQ ID NO: 86.

In some aspects, the VH of the second T cell binding arm of the tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56 or 83.

In some aspects, a tetravalent T cell engaging molecule comprising the trivalent T cell engaging molecule further comprises (d) a second T cell binding arm. In some aspects, the second T cell binding arm comprises a VL comprising a VL-CDR1 set forth in SEQ ID NO: 51; a VL-CDR2 set forth in SEQ ID NO: 52; and a VL-CDR3 set forth in SEQ ID NO: 53, and a VH comprising a VH-CDR1 set forth in SEQ ID NO: 48; a VH-CDR2 set forth in SEQ ID NO: 49; and a VH-CDR3 set forth in SEQ ID NO: 50. In some aspects, the VL of the second T cell binding arm comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54, and wherein the VH of the second T cell binding arm comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56. In some aspects, the trivalent or tetravalent T cell engaging molecule binds an epitope on an extracellular loop of STEAP2.

In some aspects, the trivalent or tetravalent T cell engaging molecule comprises a heavy chain comprising the heavy chain constant region of the second antigen binding arm and the first T cell binding arm comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 81. In some aspects, the heavy chain comprising the heavy chain constant region of the first antigen binding arm and the second T cell binding arm comprises an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 87. In some aspects, the trivalent T cell engaging molecule comprises SEQ ID NO: 25, 35, 81, 25, and 93. In some aspects, the tetravalent T cell engaging molecule comprises SEQ ID NOs: 25, 35, 81, 25, and 87. In some aspects, the tetravalent T cell engaging molecule comprises SEQ ID NOs: 151, 152, 153, 154, and 152. In some aspects, the tetravalent T cell engaging molecule comprises SEQ ID NO: 151, two copies of SEQ ID NO: 152, SEQ ID NO: 153, and SEQ ID NO: 154.

In some aspects, the T cell engaging molecule is encoded by one or more nucleic acid(s). In some aspects, a vector comprises the nucleic acid(s). In some aspects, an isolated host cell comprises the nucleic acid(s) or the vector. In other aspects, the T cell engaging molecule is formulated with a pharmaceutically acceptable carrier. The present disclosure also concerns the treatment of disease. In some aspects, the disclosure concerns a method of treating a disease in a patient in need thereof, the method comprising administering to the patient an effective amount of the T cell engaging molecule. In some aspects, the disease is cancer. In some aspects, the disease is prostate cancer. In some aspects, the T cell engaging molecule is formulated for use as a medicament. In some aspects, the T cell engaging molecule is for use in the treatment of cancer. In some aspects, the T cell engaging molecule is used in the manufacture of a medicament for the treatment of cancer.

### BRIEF DESCRIPTION OF THE DRAWINGS/FIGURES

FIG. 1A shows a schematic of a T cell engaging molecule (TED). FIG. 1B shows a schematic of a T cell engaging molecule (TED2). FIG. 1C shows a schematic of a trivalent T cell engaging molecule (TED3). FIG. 1D shows a schematic of a tetravalent T cell engaging molecule (TED4).

FIG. 2 shows a ribbon diagram of a kappa and a CH1 chain interacting surface of a binding arm of a T cell engager.

FIG. 3 shows a ribbon diagram of a lambda and a CH1 chain interacting surface of a binding arm of a T cell engager.

FIG. 4A shows binding of trivalent STEAP2 T cell engager molecules TED3 30D12 G96P/CD3 K29-SN75_3 and TED3 30D12 G96P/CD3 K29-SN75_V12 to C4-2 cells compared to TED3 Nip228_V12 controls. FIG. 4B shows CD8 binding of STEAP2 trivalent T cell engaging molecules TED3 30D12 G96P/CD3 K29-SN75_3 and TED3 30D12 G96P/CD3 K29-SN75_V12. FIG. 4C shows binding of tetravalent STEAP2 T cell engager molecule 40A3LO12-TED4-K17/E75 to C4-2 cells. FIG. 4D shows CD8 binding of STEAP2 tetravalent T cell engaging molecule 40A3LO12-TED4-K17/E7. FIG. 4E shows cytotoxicity of STEAP2 trivalent T cell engager molecules 30D12-TED3 K29/SN75 and 30D12-TED3 K29/SN75 V12 compared to 30D12-TED3-control-CD3, 30D12-TED3-control-CD3 V12, and TED3 NIP228/control-CD3/CD8 V12 controls. FIG. 4F shows CD8+ T cell activation by trivalent STEAP2 T cell engaging molecules 30D12-TED3 K29/SN75 and 30D12-TED3 K29/SN75 V12 compared to 30D12-TED3-control-CD3, 30D12-TED3-control-CD3 V12, and TED3 NIP228/control-CD3/CD8 V12 controls. FIG. 4G shows CD4+ T cell activation by trivalent STEAP2 T cell engaging molecules 30D12-TED3 K29/SN75 and 30D12-TED3 K29/SN75 V12 compared to 30D12-TED3 control-CD3, 30D12-TED3 control-CD3 V12, and TED3 NIP228/control-CD3/CD8 V12 controls. FIG. 4H shows STEAP2 T cell engaging molecule 40A3LO12-TED2-K29/SN75 demonstrated high cytotoxicity against high STEAP2 expressing cells LNCap, C4-2, VCaP, 22Rv1 cells and low cytotoxicity against low STEAP2 expressing DU145 cells n. FIG. 4I shows trivalent STEAP2 T cell engaging molecule 30D12-TED3-K29/SN75 demonstrated high cytotoxicity against high STEAP2 expressing cells LNCap, C4-2, VCaP, 22Rv1 cells and low cytotoxicity against low STEAP2 expressing DU145 cells. FIG. 4J shows the EC50 of the STEAP2 T cell engaging molecules and the trivalent STEAP2 T cell engaging molecule in cell lines with different levels of STEAP2 expression. FIG. 4K shows STEAP2 receptor density on different cell lines. FIG. 4L shows cytotoxicity and IL-6 and TNFα cytokine release induced by STEAP2 T cell engaging molecule 40A3LO12-TED2-K29/SN75 and trivalent STEAP2 T cell engaging molecule 30D12-TED3-K29/SN75 in LNCaP, C4-2 and VCaP cell lines.

FIG. 5A shows activation of CD4 and CD8 T cells by a STEAP2 T cell engaging molecule (TED) and a trivalent (TED3) STEAP2 CD8-guided T cell engaging molecule. FIG. 5B shows T-cell activation by a bivalent STEAP2 T cell engaging molecule (TED2) and various tetravalent (TED4) STEAP2 CD8-guided T cell engaging molecules. FIG. 5C shows that 30D12-TED3-K29/SN75 triggered reduced IL-6 release compared to 30D12-TED-K29/SN75 molecule at similar levels of cytotoxicity. FIG. 5D shows lower levels of IL-6 release were also observed for all variants of TED4 T cell engagers at similar levels of cytotoxicity compared to 40A3LO12-TED2-K29/SN75.

FIG. 6A shows the percent suppression of cytolysis at different Treg to T effector cell ratios by TED2 T cell engaging molecule and a TED3 trivalent STEAP2 T cell engaging molecule. FIG. 6B shows Treg activation of untreated cells and cells treated with a T cell engaging molecule (TED) and a trivalent (TED3) STEAP2 CD8-guided T cell engaging molecule.

FIG. 7A shows fluorescence of mKate-labeled C4-2 spheroid tumors over time in the absence (control) and presence of trivalent STEAP2 T cell engaging molecules (TED3) at increasing concentrations. FIG. 7B showed tumor cell killing of the mKate2-expressing C4-2 spheroids by PBMCs was also observed with all variants of TED4 T cell engagers and was increased compared to 40A3LO12-TED2-K29/SN75.

FIG. 8A shows tumor volume in human PBMC engrafted mice implanted with LNCaP tumor cells and either untreated or treated with untargeted trivalent T cell engaging molecules (NIP228-TED3-K29/SN75 V12) or trivalent (30D12-TED3-K29/SN75 V12) STEAP2 CD8-guided T cell engager molecules. FIG. 8B shows tumor volume in human PBMC engrafted mice implanted with C4-2 tumor cells and either untreated or treated with TED2 T cell engaging molecule or trivalent STEAP2 CD8-guided T cell engaging molecules. FIG. 8C shows bioluminescence images of mice engrafted with human PBMC and i.v. injected with luciferase-expressing C4-2 tumor cells and either untreated (PBS) or treated with trivalent STEAP2 CD8-guided T cell engaging molecules. FIG. 8D shows a graph of the bioluminescence of the mice in FIG. 8C.

FIG. 9 shows TNF-α levels in mice implanted with luciferase-expressing C4-2 tumor cells, and engrafted with human PBMC from two different donors and either untreated (PBS) or treated with STEAP2 T cell engaging molecules with a CD3 control binding arm (30D12-TED-CD3 control), STEAP2 T cell engaging molecules with a modified CD3 binding arm (30D12-TED-KN29/SN75), or trivalent STEAP2 CD8-guided T cell engaging molecules with a modified CD3 binding arm (30D12-TED3-K29/SN75 V12).

FIG. 10 shows tumor volume in mice subcutaneously implanted with a mix of human PBMCs and C4-2 tumor cells and either untreated (PBS) or treated with 40A3LO12-TED2-K29/SN75 or 30D12-TED3-K29/SN75 V12 molecules.

FIG. 11 shows tumor volume in human immune stem cell-engrafted mice subcutaneously implanted with C4-2 tumor cells and either untreated or treated with 30D12-TED3-K29/SN75 V12 molecules.

FIG. 12 shows the correlation between EC50 of cytolysis of tetravalent STEAP2 T cell engaging molecule 40A3LO12-TED4-K17/E75 and level of STEAP2 cell surface expression (antigen binding capacity, ABC) across 8 tumor cell lines.

FIG. 13 shows that addition of regulatory T cells (Treg) into co-cultures of effector T cells and tumor cells reduces granzyme B production in CD8 T cells in response to a non-CD8-guided T cell engaging molecule but no impact of Tregs in seen in response to CD8-guided 40A3LO12-TED4-K17/E75.

FIG. 14 shows sustained cytotoxicity of PBMCs after up to 4 rounds of co-culture with tumor cells in the presence of 40A3LO12-TED4-K17/E75.

FIG. 15 shows cytotoxicity of STEAP2-negative cells occurs only when co-cultured with various amounts of STEAP2-positive cells.

FIGS. 16A and B show cytotoxicity of a prostate cancer patient-derived xenograft organoid when co-cultured with T cells in the presence of 40A3LO12-TED4-K17/E75. No cytotoxicity was observed in response to an untargeted NIP228-TED4-K17/E75 molecule.

FIG. 16C shows increased activation of CD8+ T cells compared to CD4+ T cells at the end of this assay.

FIG. 17 shows increased IFNg release in primary prostate tumour slices treated with 40A3LO12-TED4-K17/E75.

FIG. 18A shows tumor volume in human PBMC engrafted mice implanted with 22Rv1 tumor cells and either untreated or treated with untargeted T cell engaging molecules (NIP228-TED4-K17/E75) or tetravalent (40A3LO12-TED4-K17/E75) STEAP2 CD8-guided T cell engager molecules. FIG. 18B shows unchanged levels of CD69 and CD25 on T cells in the blood of these animals after receiving 3 doses of 40A3LO12-TED4-K17/E75. FIG. 18C shows increased levels of CD69 and CD25 on CD8+ T cells but not CD4+ T cells in the tumour of these animals after receiving 3 doses of 40A3LO12-TED4-K17/E75.

FIG. 19 shows tumor burden (as measured by bioluminescent imaging) in human PBMC engrafted mice implanted intratibially with luciferase-expressing C4-2 tumor cells and either untreated or treated with untargeted T cell engaging molecules (NIP228-TED4-K17/E75) or tetravalent (40A3LO12-TED4-K17/E75) STEAP2 CD8-guided T cell engager molecules.

FIG. 20 shows tumor volume in human PBMC engrafted mice implanted with fragments of a prostate cancer patient-derived xenograft model and either untreated or treated with tetravalent (40A3LO12-TED4-K17/E75) STEAP2 CD8-guided T cell engager molecules.

### DETAILED DESCRIPTION

Provided are T cell engaging molecules that bind an antigen on a target cell, e.g., human six transmembrane epithelial antigen of prostate-2 (STEAP2) on a cancer cell, and a T cell antigen, e.g., a cluster of differentiation 3 (CD3) and/or CD8 protein. Further provided are methods of making and using the T cell engaging molecules. In some aspects, the T cell engaging molecules bind one or more antigens, e.g. one or more epitopes on a STEAP2 of a cancer cell, and one or more T cell proteins, e.g., a CD3 and/or CD8. Without wanting to be bound by theory, it is hypothesized that the number and efficacy of target protein and T cell interactions afforded by the T cell engaging molecules described herein enables an increase in target cell killing while reducing the risk of inducing a cytokine release syndrome.

### i. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002, CRC Press; The Dictionary of Cell and Molecular Biology, 3^{rd} ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

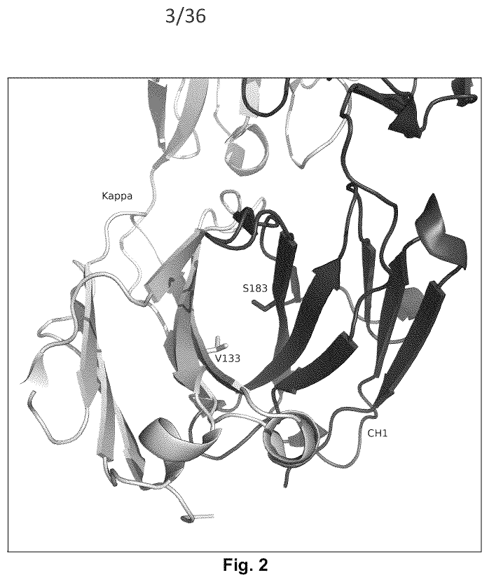

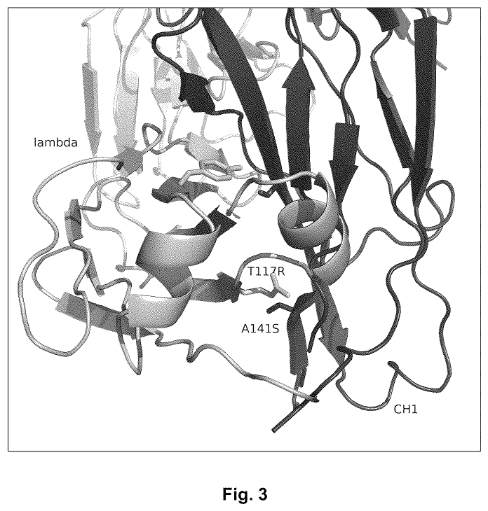

The term “T cell engaging molecule,” as used herein, refers to any molecule that can target an antigen on a target cell, e.g., a STEAP2 on a cancer cell, and also bind a protein expressed on a T cell, e.g., a CD3 and/or CD8 protein. A T cell engaging molecule can comprise at least one antigen binding portion, or antigen binding arm, and at least one T cell binding portion, or T cell binding arm. The term “antigen binding portion” and “antigen binding arm” are used interchangeably. A T cell engaging molecule can include portions of monoclonal antibodies, chimeric antibodies, humanized antibodies and human antibodies. For example, T cell engaging molecules can comprise an antigen binding portion or arm of an antibody, e.g., an anti-STEAP2 antibody and/or an anti-CD3 antibody and/or an anti-CD8 antibody and include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR); (vii) a combination of two or more isolated CDRs which can optionally be joined by a synthetic linker; or (viii) single chain Fv (scFv). Antibody fragments are obtained using conventional techniques known to those with skill in the art, and the fragments are screened for utility in the same manner as are intact antibodies. Antigen binding portions or arms can be produced by recombinant DNA techniques, or by enzymatic or chemical cleavage of intact immunoglobulins. Ribbon diagrams of a kappa and a CH1 chain interacting surface of a binding arm of a T cell engager and of a lambda and a CH1 chain interacting surface of a binding arm of a T cell engager are shown in Figures. 2 and 3, respectively.

The terms “antigen binding arm” and “antigen binding portion,” as used herein, refer to the part of a molecule that binds to all or part of the target epitope and generally comprises six complementarity-determining regions (CDRs); three in the VH region: HCDR1, HCDR2 and HCDR3, and three in the VL region: LCDR1, LCDR2, and LCDR3. The six CDRs together define the paratope of the antigen binding portion or arm, which is the part of the antigen binding portion or arm which binds to the target epitope. The antigen binding portion or arm can also comprises just the three heavy chain CDRs. The term “epitope” as used herein refers to the part of an antigen that an antigen binding arm binds to. A monoclonal monospecific IgG antibody molecule contains two antigen binding arms, each of which are able to bind the same epitope (i.e. it is bivalent for a single epitope).

An example of a bispecific antibody format that incorporates some of these modifications to improve efficient production of these molecules is a “DuetMab” described in Mazor 2015 and WO 2013/096291. DuetMab antibody molecules uses knobs-into-holes technology for heterodimerization of two distinct heavy chains and increases the efficacy of cognate heavy and light chain pairing by replacing the native disulfide bond in one of the CH1-CL interfaces with an engineered disulfide bond.

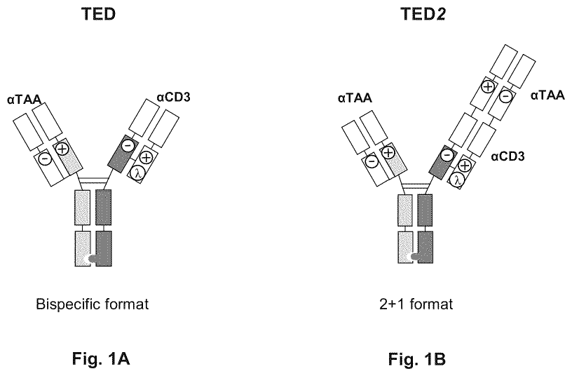

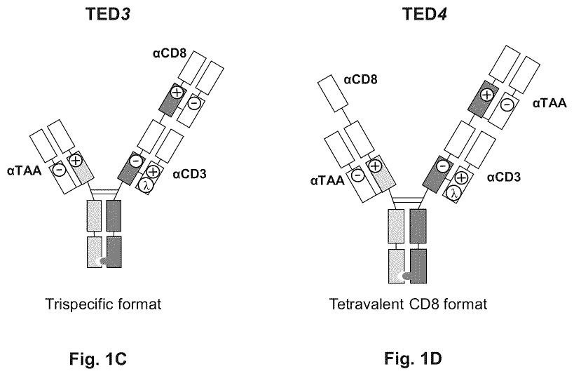

The term “valent,” as used herein, refers to the presence of a specified number of antigen binding portions or arms in the T cell engaging molecule that bind an epitope. For example, the term “trivalent,” as used herein, refers to a T cell engaging molecule that has three portions or arms that bind an antigen. The term “tetravalent,” as used herein, refers to a T cell engaging molecule that has four portions or arms that bind an antigen. The antigen binding portions or arms of a trivalent and/or tetravalent T cell engaging molecule can bind to a same antigenic molecule, can bind a same epitope on an antigenic molecule, can bind different epitopes on a same antigenic molecule and/or can bind different epitopes on different antigenic molecules. In some aspects of a bivalent molecule described herein, a single antigen binding domain binds to CD3 and a second antigen binding domain binds to another target, and is referred to as a Bispecific T-cell Engager DuetMab (“TED”; see FIG. 1A). In some aspects of a trivalent molecule described herein, a single antigen binding domain binds to CD3 in the trivalent antibody, and two additional antigen binding domains bind to other targets, and is referred to as a Bispecific T-cell Engager DuetMab (“TED2”; see FIG. 1B). In some aspects of a trivalent molecule described herein, a single antigen binding domain binds to CD3 in the trivalent antibody, a single antigen binding domain binds to CD8, and a third antigen binding domain binds to another target, which is referred to as a Trispecific T-cell engager DuetMab (“TED3”; see FIG. 1C). In some aspects of a tetravalent molecule described herein, a single antigen binding domain binds to CD3 in the tetravalent molecule, a single antigen binding domain binds to CD8, and a third and fourth antigen binding domain each bind to another target, or the same target, and may be referred to as a Tetraspecific T-cell engager DuetMab (“TED4”; see FIG. 1D). In some aspects, the TED4 format comprises two antigen binding domains that are capable of binding to the same target.

A natural evolution of bispecific antibodies has been the introduction of trispecific antibodies, which The term “linker,” as used herein, refers to a peptide chain of at least two amino acids, e.g., a glycine and/or a serine that connects two polypeptide chains, e.g., a heavy chain of an antigen binding arm and a heavy chain of a T cell binding arm. The linker can comprise one or more glycine and/or serine amino acids. The terms “linked” and “attached” and “fused” as used herein, refer to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production. For example, a linker comprises at least one “GGGGS.”

In some aspects, the linkage between a binding domain and an Fc domain includes a standard hinge region. In some aspects, the hinge region is an IgG1 hinge comprising the sequence DKTHTCPPCPAPE (SEQ ID NO: 155) between the CH1 domain of the antigen binding arm and the CH2 domain of the Fc region.

The phrase “capable of being disulfide linked,” as used herein, refers to two polypeptide chains that contain at least one cysteine each in a location such that a disulfide bridge can be formed between the two polypeptide chains when they are present in a T cell engaging molecule.

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant domain. The light chain constant domain is comprised of one domain (abbreviated herein as CL). The VH and VL regions can be further subdivided into regions of hypervariability, termed complementarity determining regions (CDR), interspersed with regions that are more conserved, termed framework regions (FR). The VH region and VL region comprise framework regions (FRs) on either side of each CDR, which provide a scaffold for the CDRs. From N-terminus to C-terminus, VH regions comprise the following structure: N term-[HFR1]-[HCDR1]-[HFR2]-[HCDR2]-[HFR3]-[HCDR3]-[HFR4]-C term; and VL regions comprise the following structure: N term-[LFR1]-[LCDR1]-[LFR2]-[LCDR2]-[LFR3]-[LCDR3]-[LFR4]-C term. An antibody can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Antibodies, e.g., IgG1, exist in several allotypes, which differ from each other in at most a few amino acids. Antibodies include, by way of example, both naturally-occurring and non-naturally-occurring antibodies; monoclonal and polyclonal antibodies; chimeric and humanized antibodies; human and nonhuman antibodies and wholly synthetic antibodies.

The variable regions of the heavy and light chains contain a binding domain (also called a paratope) that interacts with an antigen molecule (or an epitope on the antigen molecule, e.g., an epitope on a CD3 molecule). The constant regions of the antibodies can mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU numbering (Edelman, 2007). A light chain associates with a VH and CH1 in the heavy chain to form an “antigen binding arm” and the variable domains in the antigen binding arm interact to form the paratope that binds the antigen. Light chains in natural antibodies are either “lambda (λ)” or “kappa (κ)” light chains, which differ in terms of their amino acid sequence. Light chains of the trivalent and/or tetravalent T cell engager molecules described herein can also be chimeric light chains, e.g. contain a CLλ and a VLκ.

Antibodies and methods for their construction and use are well-known in the art and are described in, for example, Holliger & Hudson, Nature Biotechnology 23(9):1126-1136 (2005). In view of today's techniques in relation to monoclonal antibody technology, antibodies can be prepared to most targets. It is possible to take monoclonal and other antibody molecules and use techniques of recombinant DNA technology to produce other antibody, chimeric molecules, and/or T cell engaging molecules. Such techniques may involve introducing CDRs or variable regions of one antibody into a different antibody molecule or introducing attaching CDRs or variable regions to an antibody molecule.

As used herein, the term “affinity” refers to a measure of the strength of the binding of an antigen or target (such as an epitope) to its cognate binding domain (such as a paratope). As used herein, the term “avidity” refers to the overall stability of the complex between a population of epitopes and paratopes (i.e., antigens and antigen binding arms).

The term “epitope” refers to a site on an antigen (e.g., STEAP2, CD3 or CD8) to which a trivalent and/or tetravalent T cell engaging molecule can bind. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “binds to the same epitope” with reference to two or more antigen binding moieties means that the antigen binding moieties bind to the same segment of amino acid residues. Antigen binding moieties that “compete with another antibody for binding to a target” refer to antigen binding moieties that inhibit (partially or completely) the binding of the other antibody to the target.

As used herein, the terms “specific binding,” “selective binding,” “selectively binds,” and “specifically binds,” refer to an antigen binding portion or arm binding to an epitope on a predetermined antigen. Typically, the antigen binding portion or arm (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using a predetermined antigen, e.g., human STEAP2 or CD3 or CD8, as the analyte and the T cell engaging molecule as the ligand, or Scatchard analysis of binding of the T cell engaging molecule to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, a T cell engaging molecule that “specifically binds to human STEAP2” refers to an antigen binding portion or arm that binds to human STEAP2 with a K_{D} of 10⁻⁷ M or less, such as approximately less than 10⁻⁸ M, 10⁻⁹ M or 10⁻¹⁰ M or even lower.

The term “polypeptide,” as used herein, is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with, any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, disulfide bond formation, proteolytic cleavage, or modification by non-naturally occurring amino acids. The term “peptide,” as used herein, encompasses full length peptides and fragments, variants or derivatives thereof. A “peptide” as used herein can be part of a fusion polypeptide comprising additional components such as, e.g., an albumin or PEG moiety, to increase half-life. A peptide as used herein can also be derivatized in a number of different ways. A peptide can comprise modifications including e.g., conjugation of a palmitoyl group.

The term “conservative amino acid substitution,” as used herein, refers to a substitution of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Further, a predicted nonessential amino acid residue in an antigen binding arm can be replaced with another amino acid residue from the same side chain family.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The term “nucleic acid molecule,” as used herein, is intended to include DNA molecules and RNA molecules. A nucleic acid molecule can be single-stranded or double-stranded, and can be cDNA.

The terms “polynucleotide,” and “nucleic acid molecule,” as used herein, are intended to include DNA molecules and RNA molecules. A polynucleotide or nucleic acid molecule can be single-stranded or double-stranded, and can be cDNA.

The term “promoter,” as used herein, refers to a DNA sequence recognized by the machinery of a cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible expression by external signals or agents; such elements can be located in the 5′ or 3′ regions of the native gene. In some aspects, the promoter can be a constitutively active promoter, a cell-type specific promoter, or an inducible promoter.

The term “IRES,” as used herein, refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). Under translational control of an IRES translation proceeds in a cap-independent manner.

The term “termination signal sequence,” as used herein, can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The terms “operatively linked,” “operatively inserted,” “operatively positioned,” “under control” or “under transcriptional control,” as used herein, mean that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into a cell is positioned adjacent a DNA sequence which directs transcription and translation of the introduced DNA (i.e., facilitates the production of, e.g., a polypeptide encoded by a DNA of interest).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

The terms “subject,” “individual,” or “patient,” as used herein, refer to any organism to which a composition disclosed herein, e.g., a T cell engaging molecule can be administered, e.g., for experimental, diagnostic, prophylactic, and/or therapeutic purposes. Typical subjects include any animal (e.g., mammals such as mice, rats, rabbits, non-human primates, and humans). Mammalian subjects include, for example, humans, non-human primates, dogs, cats, guinea pigs, rabbits, rats, mice, horses, cattle, bears, and so on. A subject can seek or be in need of treatment, require treatment, be receiving treatment, be receiving treatment in the future, or be a human or animal who is under care by a trained professional for a particular disease or condition.

The terms “treat,” “treatment,” or “treatment of,” as used herein, refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

The term “immune response” is as understood in the art generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

The term “immunotherapy,” as used herein refers to the treatment of a subject afflicted with, or at risk of contracting or suffering a recurrence of, a disease by a method comprising inducing, enhancing, suppressing or otherwise modifying the immune system or an immune response.

The term “cancer,” as used herein, refers to a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream.

The terms “effective amount,” “therapeutically effective amount,” and a “sufficient amount” of, e.g., a T cell engaging molecule or a composition described herein refer to a quantity sufficient to, when administered to a subject including a human effect beneficial or desired results, including alleviation of symptoms; diminishment of the extent of a condition, disorder, or disease; attainment of a stabilized (i.e., not worsening) state of a condition, disorder, or disease; delay in onset or slowing of a condition, disorder, or disease progression; amelioration of a condition, disorder, or disease state or remission (whether partial or total), whether detectable or undetectable; amelioration of at least one measurable physical parameter, not necessarily discernible by a patient; or enhancement or improvement of a condition, disorder, or disease. In some aspects, treatment includes eliciting a clinically significant response without excessive levels of side effects. As such, a “therapeutically effective amount” or synonyms thereof depend on the context in which they are applied. In some aspects, a therapeutically effective amount of an agent (e.g., a T cell engaging molecule or a composition described herein) is an amount that results in a beneficial or desired result in a subject as compared to a control that does not receive the agent. The amount of a given agent (e.g., a T cell engaging molecule or a composition) will vary depending upon various factors, such as the given agent, the pharmaceutical formulation, the route of administration, the type of disease or disorder, the identity of the subject (e.g., age, sex, and/or weight) or host being treated, and the like.

The term “prophylactically effective amount,” as used herein, refers to an amount of an agent (e.g., a T cell engaging molecule or a composition) that delays, forestalls the onset, or blocks the onset or development or progression of a condition or disease for a period of time, including weeks, months, or years. The prophylactically effective amount can vary depending on the characteristics of an agent; how the agent is administered; the degree of risk of disease; and the history, age, weight, family history, genetic makeup of a subject; the types of preceding or concomitant treatments, if any; and other individual characteristics of the patient to be treated.

As used herein, the terms “ug” and “uM” are used interchangeably with “μg” and “μM,” respectively.

Various aspects described herein are described in further detail in the following subsections.

### II. Antigen Binding Arm

An antigen binding arm of the T cell engaging molecules described herein binds STEAP2. In some aspects, a T cell engaging molecule comprises one STEAP2 binding arm. In some aspects, a T cell engaging molecule binds two STEAP2 antigens.

In some aspects, an antigen binding arm of a T cell engaging molecule described herein can be of any format including an Fab, Fab′, F(ab′)2, Fd, Fv, single-chain fragment variable (scFv), single chain antibody, VHH, vNAR, nanobody (single-domain antibody), or any combination thereof. In some aspects, the antigen binding arm comprises a Fab.

In some aspects, an antigen binding arm comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3.

In some aspects, an antigen binding arm comprises a VH-CDR1 comprising an amino acid sequence selected from SEQ ID Nos: 1, 9, 17, 103, 111, 127, 135, and 143. In some aspects, an antigen binding arm comprises a VH-CDR2 comprising an amino acid sequence selected from SEQ ID Nos: 2, 10, 18, 104, 112, 128, 136, and 144. In some aspects, an antigen binding arm comprises a VH-CDR3 comprising an amino acid sequence selected from SEQ ID Nos: 3, 11, 19, 94, 96, 98, 105, 113, 129, 137, and 145.

In some aspects, an antigen binding arm comprises a VL-CDR1 comprising an amino acid sequence selected from SEQ ID Nos: 4, 12, 20, 100, 108, 130, 138, and 146. In some aspects, an antigen binding arm comprises a VL-CDR2 comprising an amino acid sequence selected from SEQ ID Nos: 5, 13, 21, 101, 109, 131, 139, and 147. In some aspects, an antigen binding arm comprises a VL-CDR3 comprising an amino acid sequence selected from SEQ ID Nos: 6, 14, 22, 102, 110, 132, 140, and 148.

In some aspects, an antigen binding arm comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, an antigen binding arm comprises a VH-CDR1 comprising the amino acid sequence set forth in SEQ ID NO: 9, a VH-CDR2 comprising the amino acid sequence set forth in SEQ ID NO: 10, a VH-CDR3 comprising the amino acid sequence set forth in SEQ ID NO: 11, a VL-CDR1 comprising the amino acid sequence set forth in SEQ ID NO: 12, a VL-CDR2 comprising the amino acid sequence set forth in SEQ ID NO: 13, and a VL-CDR3 comprising the amino acid sequence set forth in SEQ ID NO: 14.

In some aspects, an antigen binding arm comprises a VH-CDR1 comprising the amino acid sequence set forth in SEQ ID NO: 17, a VH-CDR2 comprising the amino acid sequence set forth in SEQ ID NO: 18, a VH-CDR3 comprising the amino acid sequence set forth in SEQ ID NO: 19, a VL-CDR1 comprising the amino acid sequence set forth in SEQ ID NO: 20, a VL-CDR2 comprising the amino acid sequence set forth in SEQ ID NO: 21, and a VL-CDR3 comprising the amino acid sequence set forth in SEQ ID NO: 22.

In some aspects, an antigen binding arm comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 94, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, an antigen binding arm comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 96, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, an antigen binding arm comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 98, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, an antigen binding arm comprises a VH-CDR1 comprising the amino acid sequence set forth in SEQ ID NO: 103, a VH-CDR2 comprising the amino acid sequence set forth in SEQ ID NO: 104, a VH-CDR3 comprising the amino acid sequence set forth in SEQ ID NO: 105, a VL-CDR1 comprising the amino acid sequence set forth in SEQ ID NO: 100, a VL-CDR2 comprising the amino acid sequence set forth in SEQ ID NO: 101, and a VL-CDR3 comprising the amino acid sequence set forth in SEQ ID NO: 102.

In some aspects, an antigen binding arm comprises a VH-CDR1 comprising the amino acid sequence set forth in SEQ ID NO: 111, a VH-CDR2 comprising the amino acid sequence set forth in SEQ ID NO: 112, a VH-CDR3 comprising the amino acid sequence set forth in SEQ ID NO: 113, a VL-CDR1 comprising the amino acid sequence set forth in SEQ ID NO: 108, a VL-CDR2 comprising the amino acid sequence set forth in SEQ ID NO: 109, and a VL-CDR3 comprising the amino acid sequence set forth in SEQ ID NO: 110.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 7. In some aspects, an antigen binding arm comprises a VH comprising an amino acid sequence set forth in SEQ ID NO: 7.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 15. In some aspects, an antigen binding arm comprises a VH comprising an amino acid sequence set forth in SEQ ID NO: 15.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 23. In some aspects, an antigen binding arm comprises a VH comprising an amino acid sequence set forth in SEQ ID NO: 23.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 95. In some aspects, an antigen binding arm comprises a VH comprising an amino acid sequence set forth in SEQ ID NO: 95.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 97. In some aspects, an antigen binding arm comprises a VH comprising an amino acid sequence set forth in SEQ ID NO: 97.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 99. In some aspects, an antigen binding arm comprises a VH comprising an amino acid sequence set forth in SEQ ID NO: 99.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 106. In some aspects, an antigen binding arm comprises a VH comprising an amino acid sequence set forth in SEQ ID NO: 106.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 114. In some aspects, an antigen binding arm comprises a VH comprising an amino acid sequence set forth in SEQ ID NO: 114.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 133. In some aspects, an antigen binding arm comprises a VH comprising an amino acid sequence set forth in SEQ ID NO: 133.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 141. In some aspects, an antigen binding arm comprises a VH comprising an amino acid sequence set forth in SEQ ID NO: 141.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 149. In some aspects, an antigen binding arm comprises a VH comprising an amino acid sequence set forth in SEQ ID NO: 149.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 8. In some aspects, an antigen binding arm comprises a VL comprising an amino acid sequence set forth in SEQ ID NO: 8.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 16. In some aspects, an antigen binding arm comprises a VL comprising an amino acid sequence set forth in SEQ ID NO: 16.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 24. In some aspects, an antigen binding arm comprises a VL comprising an amino acid sequence set forth in SEQ ID NO: 24.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 107. In some aspects, an antigen binding arm comprises a VL comprising an amino acid sequence set forth in SEQ ID NO: 107.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115. In some aspects, an antigen binding arm comprises a VL comprising an amino acid sequence set forth in SEQ ID NO: 115.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 134. In some aspects, an antigen binding arm comprises a VL comprising an amino acid sequence set forth in SEQ ID NO: 134.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 142. In some aspects, an antigen binding arm comprises a VL comprising an amino acid sequence set forth in SEQ ID NO: 142.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 150. In some aspects, an antigen binding arm comprises a VL comprising an amino acid sequence set forth in SEQ ID NO: 150.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, a T cell engaging molecule comprises an antigen binding arm comprising a VH comprising the amino acid sequence set forth in SEQ ID NO: 7, and a VL comprising the amino acid sequence set forth in SEQ ID NO: 8.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 15, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, a T cell engaging molecule comprises an antigen binding arm comprising a VH comprising the amino acid sequence set forth in SEQ ID NO: 15, and a VL comprising the amino acid sequence set forth in SEQ ID NO: 16.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 23, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 24. In some aspects, a T cell engaging molecule comprises an antigen binding arm comprising a VH comprising the amino acid sequence set forth in SEQ ID NO: 23, and a VL comprising the amino acid sequence set forth in SEQ ID NO: 24.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 95, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, a T cell engaging molecule comprises an antigen binding arm comprising a VH comprising the amino acid sequence set forth in SEQ ID NO: 95, and a VL comprising the amino acid sequence set forth in SEQ ID NO: 8.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 97, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, a T cell engaging molecule comprises an antigen binding arm comprising a VH comprising the amino acid sequence set forth in SEQ ID NO: 97, and a VL comprising the amino acid sequence set forth in SEQ ID NO: 16.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 99, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 24. In some aspects, a T cell engaging molecule comprises an antigen binding arm comprising a VH comprising the amino acid sequence set forth in SEQ ID NO: 99, and a VL comprising the amino acid sequence set forth in SEQ ID NO: 24.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 106, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 107. In some aspects, a T cell engaging molecule comprises an antigen binding arm comprising a VH comprising the amino acid sequence set forth in SEQ ID NO: 106, and a VL comprising the amino acid sequence set forth in SEQ ID NO: 107.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 114, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 115. In some aspects, a T cell engaging molecule comprises an antigen binding arm comprising a VH comprising the amino acid sequence set forth in SEQ ID NO: 114, and a VL comprising the amino acid sequence set forth in SEQ ID NO: 115.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 133, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 134. In some aspects, a T cell engaging molecule comprises an antigen binding arm comprising a VH comprising the amino acid sequence set forth in SEQ ID NO: 133, and a VL comprising the amino acid sequence set forth in SEQ ID NO: 134.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 141, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 142. In some aspects, a T cell engaging molecule comprises an antigen binding arm comprising a VH comprising the amino acid sequence set forth in SEQ ID NO: 141, and a VL comprising the amino acid sequence set forth in SEQ ID NO: 142.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 149, and a VL comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 150. In some aspects, a T cell engaging molecule comprises an antigen binding arm comprising a VH comprising the amino acid sequence set forth in SEQ ID NO: 149, and a VL comprising the amino acid sequence set forth in SEQ ID NO: 150.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149, and a VL comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 8, 16, 24, 107, 115, 134, 142, and 150. In some aspects, a T cell engaging molecule comprises an antigen binding arm comprising a VH comprising an amino acid sequence selected from SEQ ID Nos: 7, 15, 23, 95, 97, 99, 106, 114, 133, 141, and 149, and a VL comprising the amino acid sequence selected from SEQ ID NO: 8, 16, 24, 107, 115, 134, 142, and 150.

In some aspects, a T cell engaging molecule comprises an antigen binding arm comprising a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a light chain constant domain comprising an amino acid sequence selected from SEQ ID Nos: 59, 60 and 61.

In some aspects, a T cell engaging molecule comprises an antigen binding arm comprising a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 8 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 8 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 8 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 8 and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 16 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 16 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 16 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 16 and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 24 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 24 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 24 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 24 and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 107 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 107 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 107 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 107, and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 115 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 115, and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 115 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 115, and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 115 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 134 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 134, and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 142 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, an antigen binding arm comprises a VL comprising an amino acid sequence set forth in SEQ ID NO: 142 and a light chain constant domain comprising an amino acid sequence selected from SEQ ID Nos: 59, 60, and 61.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 150 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, an antigen binding arm comprises a VL comprising an amino acid sequence set forth in SEQ ID NO: 150, and a light chain constant domain comprising an amino acid sequence selected from SEQ ID Nos: 62, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 8 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 8 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 16 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 16 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 24 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 24 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 107 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 107 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 115 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 115 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 134 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 134 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 142 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 142 and a light chain constant domain comprising an amino acid sequence selected from SEQ ID Nos: 60, 61, 63, and 64.

In some aspects, a T cell engaging molecule comprises an antigen binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 150 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64. In some aspects, a T cell engaging molecule comprises an antigen binding arm comprising a VL comprising an amino acid sequence set forth in SEQ ID NO: 150 and a light chain constant domain comprising an amino acid sequence selected from SEQ ID Nos: 60, 61, 63, and 64.

In some aspects, a T cell engaging molecule comprises an antigen binding arm comprising a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, an antigen binding arm comprises a heavy chain constant region comprising an amino acid sequence selected from SEQ ID Nos: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 7 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 7 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 15 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 15 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 23 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 23 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 95 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 95 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 97 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 97 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 99 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 99 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 106 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 106 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 114 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 114 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 133 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 133 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 141 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 141 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 149 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell engaging molecule comprises an antigen binding arm comprising a VH comprising an amino acid sequence set forth in SEQ ID NO: 149 and a heavy chain constant region comprising an amino acid sequence selected from SEQ ID NO: 65-80.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 7 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 15 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 23 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 95 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 97 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 99 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 106 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 114 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 133 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 141 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises an antigen binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 149 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, 80, 82, 88, and 91.

In some aspects, a T cell engaging molecule comprises a heavy chain that comprises a STEAP2 antigen binding region fused to a CD8 binding region. In some aspects, the antigen binding arm further comprises a light chain comprising a STEAP2 binding region.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 25. In some aspects, a T cell engaging molecule comprises a STEAP2 binding arm that comprises a light chain comprising the amino acid sequence set forth in SEQ ID NO: 25.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 116. In some aspects, a T cell engaging molecule comprises a STEAP2 binding arm that comprises a light chain comprising the amino acid sequence set forth in SEQ ID NO: 116.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117. In some aspects, a T cell engaging molecule comprises a STEAP2 binding arm that comprises a light chain comprising the amino acid sequence set forth in SEQ ID NO: 117.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 118. In some aspects, a T cell engaging molecule comprises a STEAP2 binding arm that comprises a light chain comprising the amino acid sequence set forth in SEQ ID NO: 118.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 119. In some aspects, a T cell engaging molecule comprises a STEAP2 binding arm that comprises a light chain comprising the amino acid sequence set forth in SEQ ID NO: 119.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26. In some aspects, a T cell engaging molecule comprises a STEAP2 binding arm that comprises a heavy chain comprising the amino acid sequence set forth in SEQ ID NO: 26.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 120. In some aspects, a T cell engaging molecule comprises a STEAP2 binding arm that comprises a heavy chain comprising the amino acid sequence set forth in SEQ ID NO: 120.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 121. In some aspects, a T cell engaging molecule comprises a STEAP2 binding arm that comprises a heavy chain comprising the amino acid sequence set forth in SEQ ID NO: 121.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 122. In some aspects, a T cell engaging molecule comprises a STEAP2 binding arm that comprises a heavy chain comprising the amino acid sequence set forth in SEQ ID NO: 122.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 123. In some aspects, a T cell engaging molecule comprises a STEAP2 binding arm that comprises a heavy chain comprising the amino acid sequence set forth in SEQ ID NO: 123.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 124. In some aspects, a T cell engaging molecule comprises a STEAP2 binding arm that comprises a heavy chain comprising the amino acid sequence set forth in SEQ ID NO: 124.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 125. In some aspects, a T cell engaging molecule comprises a STEAP2 binding arm that comprises a heavy chain comprising the amino acid sequence set forth in SEQ ID NO: 125.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 126. In some aspects, a T cell engaging molecule comprises a STEAP2 binding arm that comprises a heavy chain comprising the amino acid sequence set forth in SEQ ID NO: 126. In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 26 and a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 25. In some aspects, a T cell engaging molecule comprises a STEAP2 binding arm that comprises a heavy chain comprising the amino acid sequence set forth in SEQ ID NO: 26 and a light chain comprising the amino acid sequence set forth in SEQ ID NO: 25.

### III. T Cell Binding Arm

A T cell engaging molecule described herein comprises at least one T cell binding arm. In some aspects, the T cell engaging molecule described herein comprises two T cell binding arms. In some aspects, the T cell engaging molecule comprises at least one T cell binding arm that binds a CD3 antigen. In some aspects, the T cell engaging molecule comprises at least one T cell binding arm that binds a CD8 antigen. In some aspects, the T cell engaging molecule comprises at least one T cell binding arm that binds a CD3 antigen and at least one T cell binding arm that binds a CD8 antigen. In some aspects, the T cell binding arm comprises a heavy variable domain and a light chain variable domain. In some aspects, the T cell binding arm comprises only a heavy chain variable domain.

In some aspects, a T cell binding arm of the T cell engaging molecules described herein can be of any format including an Fab, Fab′, F(ab′)2, Fd, Fv, single-chain fragment variable (scFv), single chain antibody, VHH, vNAR, nanobody (single-domain antibody), or any combination thereof. In some aspects, the antigen binding arm comprises a Fab. In some aspects, the antigen binding arm comprises a VHH.

In some aspects, a T cell binding arm comprises a variable heavy chain region (VH) and a variable light chain region (VL), wherein the VH comprises a VH-CDR1, a VH-CDR2, a VH-CDR3; and wherein the VL comprises a VL-CDR1, a VL-CDR2, and VL-CDR3.

In some aspects, a T cell binding arm of the T cell engaging molecule is capable of binding CD3. CD3 (cluster of differentiation 3) is a protein complex composed of four subunits, the CD3γ chain, the CD3δ chain, and two CD3ε chains. CD3 associates with the T-cell receptor and the ζ chain to generate an activation signal in T lymphocytes. T cell engaging molecules that target CD3 and a target cell antigen (or antigens), e.g., STEAP2 can force a temporary interaction between the target cell (or cells), e.g., a STEAP2 expressing cancer cell and a T cell, causing cross-linking, T-cell activation, and subsequent antigen-dependent T cell killing of the target cell. In some aspects, the T cell engaging molecule binds monovalently to the CD3 protein and the T-cell receptor is only cross-linked and activated upon binding of the target cell.

In some aspects, a T cell binding arm comprises a VH-CDR1 comprising an amino acid sequence selected from SEQ ID Nos: 36, 40, and 44. In some aspects, a T cell binding arm comprises a VH-CDR2 comprising an amino acid sequence selected from SEQ ID Nos: 37, 41, and 45. In some aspects, a T cell binding arm comprises a VH-CDR3 comprising an amino acid sequence selected from SEQ ID Nos: 38, 42, and 46.

In some aspects, a T cell binding arm comprises a VL-CDR1 comprising an amino acid sequence selected from SEQ ID Nos: 27 and 31. In some aspects, a T cell binding arm comprises a VL-CDR2 comprising an amino acid sequence selected from SEQ ID Nos: 28 and 32. In some aspects, a T cell binding arm comprises a VL-CDR3 comprising an amino acid sequence selected from SEQ ID Nos: 29 and 33.

In some aspects, a T cell binding arm comprises a VH-CDR1 comprising the amino acid sequence set forth in SEQ ID NO: 36, a VH-CDR2 comprising the amino acid sequence set forth in SEQ ID NO: 37, a VH-CDR3 comprising the amino acid sequence set forth in SEQ ID NO: 38, a VL-CDR1 comprising the amino acid sequence set forth in SEQ ID NO: 31, a VL-CDR2 comprising the amino acid sequence set forth in SEQ ID NO: 32, and a VL-CDR3 comprising the amino acid sequence set forth in SEQ ID NO: 33.

In some aspects, a T cell binding arm comprises a VH-CDR1 comprising the amino acid sequence set forth in SEQ ID NO: 36, a VH-CDR2 comprising the amino acid sequence set forth in SEQ ID NO: 37, a VH-CDR3 comprising the amino acid sequence set forth in SEQ ID NO: 38, a VL-CDR1 comprising the amino acid sequence set forth in SEQ ID NO: 27, a VL-CDR2 comprising the amino acid sequence set forth in SEQ ID NO: 28, and a VL-CDR3 comprising the amino acid sequence set forth in SEQ ID NO: 29.

In some aspects, a T cell binding arm comprises a VH-CDR1 comprising the amino acid sequence set forth in SEQ ID NO: 40, a VH-CDR2 comprising the amino acid sequence set forth in SEQ ID NO: 41, a VH-CDR3 comprising the amino acid sequence set forth in SEQ ID NO: 42, a VL-CDR1 comprising the amino acid sequence set forth in SEQ ID NO: 31, a VL-CDR2 comprising the amino acid sequence set forth in SEQ ID NO: 32, and a VL-CDR3 comprising the amino acid sequence set forth in SEQ ID NO: 33.

In some aspects, a T cell binding arm comprises a VH-CDR1 comprising the amino acid sequence set forth in SEQ ID NO: 40, a VH-CDR2 comprising the amino acid sequence set forth in SEQ ID NO: 41, a VH-CDR3 comprising the amino acid sequence set forth in SEQ ID NO: 42, a VL-CDR1 comprising the amino acid sequence set forth in SEQ ID NO: 27, a VL-CDR2 comprising the amino acid sequence set forth in SEQ ID NO: 28, and a VL-CDR3 comprising the amino acid sequence set forth in SEQ ID NO: 29.

In some aspects, a T cell binding arm comprises a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46, a VL-CDR1 comprising the amino acid sequence set forth in SEQ ID NO: 31, a VL-CDR2 comprising the amino acid sequence set forth in SEQ ID NO: 32, and a VL-CDR3 comprising the amino acid sequence set forth in SEQ ID NO: 33.

In some aspects, a T cell binding arm comprises a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46, a VL-CDR1 comprising the amino acid sequence set forth in SEQ ID NO: 27, a VL-CDR2 comprising the amino acid sequence set forth in SEQ ID NO: 28, and a VL-CDR3 comprising the amino acid sequence set forth in SEQ ID NO: 29.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 39. In some aspects, a T cell binding arm comprises a VL comprising an amino acid sequence set forth in SEQ ID NO: 39.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 43. In some aspects, a T cell binding arm comprises a VL comprising an amino acid sequence set forth in SEQ ID NO: 43.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 47. In some aspects, a T cell binding arm comprises a VL comprising an amino acid sequence set forth in SEQ ID NO: 47.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34. In some aspects, a T cell binding arm comprises a VL comprising an amino acid sequence set forth in SEQ ID NO: 34.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30. In some aspects, a T cell binding arm comprises a VL comprising an amino acid sequence set forth in SEQ ID NO: 30.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 39 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 39 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 43 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 43 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 47 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 47 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30.

In some aspects, a T cell binding arm comprises the VH-CDR1, VH-CDR2, and VH-CDR3 present in the VH region having the amino acid sequence set forth in SEQ ID NO: 39; and the VL-CDR1, VL-CDR2, and VL-CDR3 present in the VL region having the amino acid sequence set forth in SEQ ID NO: 34.

In some aspects, a T cell binding arm comprises the VH-CDR1, VH-CDR2, and VH-CDR3 present in the VH region having the amino acid sequence set forth in SEQ ID NO: 39; and the VL-CDR1, VL-CDR2, and VL-CDR3 present in the VL region having the amino acid sequence set forth in SEQ ID NO: 30.

In some aspects, a T cell binding arm comprises the VH-CDR1, VH-CDR2, and VH-CDR3 present in the VH region having the amino acid sequence set forth in SEQ ID NO: 43; and the VL-CDR1, VL-CDR2, and VL-CDR3 present in the VL region having the amino acid sequence set forth in SEQ ID NO: 34.

In some aspects, a T cell binding arm comprises the VH-CDR1, VH-CDR2, and VH-CDR3 present in the VH region having the amino acid sequence set forth in SEQ ID NO: 43; and the VL-CDR1, VL-CDR2, and VL-CDR3 present in the VL region having the amino acid sequence set forth in SEQ ID NO: 30.

In some aspects, a T cell binding arm comprises the VH-CDR1, VH-CDR2, and VH-CDR3 present in the VH region having the amino acid sequence set forth in SEQ ID NO: 47; and the VL-CDR1, VL-CDR2, and VL-CDR3 present in the VL region having the amino acid sequence set forth in SEQ ID NO: 34.

In some aspects, a T cell binding arm comprises the VH-CDR1, VH-CDR2, and VH-CDR3 present in the VH region having the amino acid sequence set forth in SEQ ID NO: 47; and the VL-CDR1, VL-CDR2, and VL-CDR3 present in the VL region having the amino acid sequence set forth in SEQ ID NO: 30.

In some aspects, a T cell engaging molecule comprises a T cell binding arm comprising a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, a T cell binding arm comprises a light chain constant domain comprising an amino acid sequence selected from SEQ ID Nos: 59, 60 and 61.

In some aspects, a T cell engaging molecule comprises a T cell binding arm comprising a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, a T cell binding arm comprises a light chain constant domain comprising an amino acid sequence selected from SEQ ID Nos: 62, 63, and 64.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 34 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 30 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 60, 61, 63, and 64.

In some aspects, a T cell engaging molecule comprises a T cell binding arm comprising a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80. In some aspects, a T cell binding arm comprises a heavy chain constant region comprising an amino acid sequence selected from SEQ ID Nos: 65-80.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 39 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 43 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 47 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 65-80.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 39 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, and 80.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 43 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, and 80.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 47 and a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 66, 67, 69, 72, 73, 74, 75, 76, 77, 78, 79, and 80.

In some aspects, a T cell engaging molecule comprises a T cell binding arm comprising a heavy chain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 81 or 82. In some aspects, a T cell engaging molecule comprises a T cell binding arm comprising a heavy chain comprising an amino acid sequence set forth in SEQ ID NO: 81 or 82.

In some aspects, one of the T cell binding arms is capable of binding CD8. CD8 (cluster of differentiation 8) is a dimer consisting of a pair of CD8 chains. The most common form of CD8 is composed of a CD8-α and CD8-β chain. CD8 serves as the co-receptor on MCHI-restricted T-cells and acts to enhance the antigen sensitivity of CD8+ T-cells by binding to a largely invariant region of MHCI at a site distinct from where the T-cell receptor binds.

In some embodiments, a T cell engaging molecule comprises a T cell binding arm that is capable of binding CD3 and a T cell binding arm is capable of binding CD8.

In some aspects, a T cell binding arm comprises a VH-CDR1 comprising an amino acid sequence selected from SEQ ID Nos: 48 and 84. In some aspects, the T cell binding arm comprises a VH-CDR2 comprising an amino acid sequence selected from SEQ ID Nos: 49 and 85. In some aspects, the T cell binding arm comprises a VH-CDR3 comprising an amino acid sequence selected from SEQ ID Nos: 50 and 86.

In some aspects, a T cell binding arm comprises a VL-CDR1 comprising an amino acid sequence set forth in SEQ ID NO: 51. In some aspects, wherein the T cell binding arm comprises a VL-CDR2 comprising an amino acid sequence set forth in SEQ ID NO: 52. In some aspects, a T cell binding arm comprises a VL-CDR3 comprising an amino acid sequence set forth in SEQ ID NO: 53.

In some aspects, a T cell binding arm comprises a VH-CDR1 comprising the amino acid sequence set forth in SEQ ID NO: 48, a VH-CDR2 comprising the amino acid sequence set forth in SEQ ID NO: 49, a VH-CDR3 comprising the amino acid sequence set forth in SEQ ID NO: 50, a VL-CDR1 comprising the amino acid sequence set forth in SEQ ID NO: 51, a VL-CDR2 comprising the amino acid sequence set forth in SEQ ID NO: 52, and a VL-CDR3 comprising the amino acid sequence set forth in SEQ ID NO: 53.

In some aspects, a T cell binding arm comprises a VH-CDR1 comprising the amino acid sequence set forth in SEQ ID NO: 84, a VH-CDR2 comprising the amino acid sequence set forth in SEQ ID NO: 85, and a VH-CDR3 comprising the amino acid sequence set forth in SEQ ID NO: 86. In some aspects, a T cell binding arm comprises the VH-CDR1, VH-CDR2, and VH-CDR3 present in the VHH region having the amino acid sequence set forth in SEQ ID NO: 83.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56. In some aspects, a T cell binding arm comprises a VH comprising an amino acid sequence set forth in SEQ ID NO: 56.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54. In some aspects, a T cell binding arm comprises a VL comprising an amino acid sequence set forth in SEQ ID NO: 54.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56 and a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54.

In some aspects, a T cell binding arm comprises the VH-CDR1, VH-CDR2, and VH-CDR3 present in the VH region having the amino acid sequence set forth in SEQ ID NO: 56; and the VL-CDR1, VL-CDR2, and VL-CDR3 present in the VL region having the amino acid sequence set forth in SEQ ID NO: 54.

In some aspects, a T cell engaging molecule comprises a T cell binding arm comprising a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, a T cell binding arm comprises a light chain constant domain comprising an amino acid sequence selected from SEQ ID Nos: 62, 63, and 64.

In some aspects, a T cell engaging molecule comprises a T cell binding arm comprising a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, a T cell binding arm comprises a light chain constant domain comprising an amino acid sequence selected from SEQ ID Nos: 59, 60 and 61.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 62, 63, and 64. In some aspects, a T cell engaging molecule comprises a T cell binding arm comprising a VL comprising an amino acid sequence set forth in SEQ ID NO: 54 and a light chain constant domain comprising an amino acid sequence selected from SEQ ID Nos: 62, 63, and 64.

In some aspects, a T cell engaging molecule comprises a T cell binding arm comprising a VL comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 59, 60, and 61. In some aspects, a T cell engaging molecule comprises a T cell binding arm comprising a VL comprising an amino acid sequence set forth in SEQ ID NO: 54 and a light chain constant domain comprising an amino acid sequence selected from SEQ ID Nos: 59, 60, and 61.

In some aspects, a T cell engaging molecule comprises a T cell binding arm comprising a heavy chain constant region comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 57, 65, 66, and 67. In some aspects, a T cell binding arm comprises a heavy chain constant region comprising an amino acid sequence selected from SEQ ID Nos: 57, 65, 66, and 67.

In some aspects, a T cell engaging molecule comprises a T cell binding arm comprising a VH comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 83. In some aspects, a T cell binding arm comprises a VH comprising an amino acid sequence set forth in SEQ ID NO: 83.

### IV. Charge Pairs

The terms “charge pair(s)” and “charge mutation(s)” are used interchangeably herein and refer to oppositely charged amino acids, e.g., a positively charged amino acid residue and a negatively charged amino acid residue, one of which is located in a light chain region (e.g. constant light chain domain) and the other in a heavy chain region (e.g. constant heavy chain region 1 (CH1)) of an antigen and/or T cell binding arm, located at positions intended to promote association of the light and heavy chains. By “lambda charge pair”, it is meant a charge pair where a positively or negatively charged amino acid residue is located in a lambda light chain (e.g. CLλ). By “kappa charge pair”, it is meant a charge pair where positively or negatively charged amino acid residue in the light chain is located in a kappa light chain (e.g. CLκ).

Without wishing to be bound by theory, it is believed that the oppositely charged amino acid residues in the charge pair increase the attraction of the heavy chain to the light chain in an immunoglobulin, and/or T cell binding arm, thereby promoting formation of the immunoglobulin and/or T cell binding arm with the correct heavy and light chain.

At least one of the amino acid residues of the charge pair can be engineered into the immunoglobulin, and/or T cell binding arm (i.e. at least one amino acid residue in the pair is not a wild-type amino acid residue). In some aspects, both amino acid residues in the charge pair are engineered into the immunoglobulin and/or T cell binding arm (i.e. both amino acid residues in the pair are not wild-type amino acid residues).

In some embodiments, the positively charged amino acid residue in the charge pair is located on the light chain and the negatively charged amino acid residue in the charge pair is located on the corresponding heavy chain. In other embodiments, the negatively charged amino acid residue is located on the light chain and the positively charged amino acid residue in the charge pair is located on the corresponding heavy chain.

The amino acid residues of the charge pair are typically naturally occurring. Naturally occurring positively charged amino acid residues according to the present disclosure include arginine, lysine and histidine. Naturally occurring negatively charged amino acid residues according to the present disclosure include glutamic acid, serine, threonine and aspartic acid. Although serine and threonine are often described in the art as ‘uncharged’, they have an isoelectric point below 6 and therefore are partially negatively charged at neutral pH. For the purposes of the charge pairs disclosed herein, serine and threonine are examples of negatively charged amino acid residues (together with glutamic acid and aspartic acid).

In some aspects, a charge pair comprises a positively charged amino acid residue selected from arginine, lysine or histidine located at one of the positions in the charge pair and a negatively charged amino acid residue selected from aspartic acid, glutamic acid, serine or threonine located at the other position in the charge pair. For example, the charge pair may comprise any one of the following pairs of amino acid residues:
arginine and aspartic acid;
arginine and glutamic acid;
arginine and serine;
arginine and threonine;
lysine and aspartic acid;
lysine and glutamic acid;
lysine and serine;
lysine and threonine;
histidine and aspartic acid;
histidine and glutamic acid;
histidine and serine; and histidine and threonine.

### Lambda Charge Pairs

Lambda charge pairs, as exemplified herein, can be introduced at several positions to improve pairing of the correct light and heavy chains in the antigen and/or T cell binding arm.

In some aspects, a lambda charge pair comprises a positively or negatively charged amino acid residue at position 117, 119, 134, 136 or 178 of the constant light chain lambda region (CLλ). In some aspects, a lambda charge pair comprises a positively or negatively charged amino acid residue at position 141, 185, 128, 145, 183, 185, 173, or 187 of the CH1 domain. As noted elsewhere, the numbering is according to EU numbering.

In some aspects, lambda charge pairs are located at one or more of the following pairs of positions:
(iv) position 117 in the CLλ and position 141 in the CH1;
(ii) position 117 in the CLλ and position 185 in the CH1;
(iii) position 119 in the CLλ and position 128 in the CH1;
(iv) position 134 in the CLλ and position 128 in the CH1;
(v) position 134 in the CLλ and position 145 in the CH1;
(vi) position 134 in the CLλ and position 183 in the CH1;
(vii) position 136 in the CLλ and position 185 in the CH1;
(viii) position 178 in the CLλ and position 173 in the CH1; and
(ix) position 117 in the CLλ and position 187 in the CH1.

In some aspects, the lambda charge pair is located at position 117 in the CLλ and position 141 in the CH1. For example, the lambda charge pair can be selected from the following list:
arginine at position 117 of the CLλ and aspartic acid at position 141 of the CH1;
arginine at position 117 of the CLλ and glutamic acid at position 141 of the CH1;
arginine at position 117 of the CLλ and serine at position 141 of the CH1;
arginine at position 117 of the CLλ and threonine at position 141 of the CH1;
lysine at position 117 of the CLλ and aspartic acid at position 141 of the CH1;
lysine at position 117 of the CLλ and glutamic acid at position 141 of the CH1;
lysine at position 117 of the CLλ and serine at position 141 of the CH1; and
lysine at position 117 of the CLλ and threonine at position 141 of the CH1.

In some aspects, a lambda charge pair is selected from any one of a. to f. of the above list. In some aspects, a lambda charge pair is selected from any one of a. to e. of the above list. In some aspects, a lambda charge pair is selected from any one of a., b., and e. of the above list. In some aspects, a lambda charge pair is a.

In some aspects, a lambda charge pair is located at position 117 in the CLλ and position 185 in the CH1. For example, a lambda charge pair can be selected from the following list:
arginine at position 117 of the CLλ and aspartic acid at position 185 of the CH1;
arginine at position 117 of the CLλ and glutamic acid at position 185 of the CH1;
arginine at position 117 of the CLλ and serine at position 185 of the CH1;
arginine at position 117 of the CLλ and threonine at position 185 of the CH1;
lysine at position 117 of the CLλ and aspartic acid at position 185 of the CH1;
lysine at position 117 of the CLλ and glutamic acid at position 185 of the CH1;
lysine at position 117 of the CLλ and serine at position 185 of the CH1; and
lysine at position 117 of the CLλ and threonine at position 185 of the CH1.

In some aspects, a lambda charge pair is located at position 119 in the CLλ and position 128 in the CH1 domain. For example, a lambda charge pair can be selected from the following list:
arginine at position 119 of the CLλ and aspartic acid at position 128 of the CH1;
arginine at position 119 of the CLλ and glutamic acid at position 128 of the CH1;
arginine at position 119 of the CLλ and serine at position 128 of the CH1;
arginine at position 119 of the CLλ and threonine at position 128 of the CH1;
lysine at position 119 of the CLλ and aspartic acid at position 128 of the CH1;
lysine at position 119 of the CLλ and glutamic acid at position 128 of the CH1;
lysine at position 119 of the CLλ and serine at position 128 of the CH1; and
lysine at position 119 of the CLλ and threonine at position 128 of the CH1.

In some aspects, a lambda charge pair is located at position 134 in the CLλ and position 128 in the CH1 domain. For example, a lambda charge pair can be selected from the following list:
arginine at position 134 of the CLλ and aspartic acid at position 128 of the CH1;
arginine at position 134 of the CLλ and glutamic acid at position 128 of the CH1;
arginine at position 134 of the CLλ and serine at position 128 of the CH1;
arginine at position 134 of the CLλ and threonine at position 128 of the CH1;
lysine at position 134 of the CLλ and aspartic acid at position 128 of the CH1;
lysine at position 134 of the CLλ and glutamic acid at position 128 of the CH1;
lysine at position 134 of the CLλ and serine at position 128 of the CH1; and
lysine at position 134 of the CLλ and threonine at position 128 of the CH1.

In some aspects, a lambda charge pair is located at position 134 in the CLλ and position 145 in the CH1 domain. For example, a lambda charge pair can be selected from the following list:
arginine at position 134 of the CLλ and aspartic acid at position 145 of the CH1;
arginine at position 134 of the CLλ and glutamic acid at position 145 of the CH1;
arginine at position 134 of the CLλ and serine at position 145 of the CH1;
arginine at position 134 of the CLλ and threonine at position 145 of the CH1;
lysine at position 134 of the CLλ and aspartic acid at position 145 of the CH1;
lysine at position 134 of the CLλ and glutamic acid at position 145 of the CH1;
lysine at position 134 of the CLλ and serine at position 145 of the CH1; and
lysine at position 134 of the CLλ and threonine at position 145 of the CH1.

In some aspects, a lambda charge pair is located at position 134 in the CLλ and position 183 in the CH1 domain. For example, a lambda charge pair can be selected from the following list:
arginine at position 134 of the CLλ and aspartic acid at position 183 of the CH1;
arginine at position 134 of the CLλ and glutamic acid at position 183 of the CH1;
arginine at position 134 of the CLλ and serine at position 183 of the CH1;
arginine at position 134 of the CLλ and threonine at position 183 of the CH1;
lysine at position 134 of the CLλ and aspartic acid at position 183 of the CH1;
lysine at position 134 of the CLλ and glutamic acid at position 183 of the CH1;
lysine at position 134 of the CLλ and serine at position 183 of the CH1; and
lysine at position 134 of the CLλ and threonine at position 183 of the CH1.

In some aspects, a lambda charge pair is a lysine at position 134 of the CLλ, and an aspartic acid or a serine at position 183 of the CH1 domain.

In some aspects, a lambda charge pair is located at position 136 in the CLλ and position 185 in the CH1 domain. For example, a lambda charge pair can be selected from the following list:
arginine at position 136 of the CLλ and aspartic acid at position 185 of the CH1;
arginine at position 136 of the CLλ and glutamic acid at position 185 of the CH1;
arginine at position 136 of the CLλ and serine at position 185 of the CH1;
arginine at position 136 of the CLλ and threonine at position 185 of the CH1;
lysine at position 136 of the CLλ and aspartic acid at position 185 of the CH1;
lysine at position 136 of the CLλ and glutamic acid at position 185 of the CH1;
lysine at position 136 of the CLλ and serine at position 185 of the CH1; and
lysine at position 136 of the CLλ and threonine at position 185 of the CH1.

In some aspects, a lambda charge pair is located at position 178 in the CLλ and position 173 in the CH1 domain. For example, a lambda charge pair can be selected from the following list:
arginine at position 178 of the CLλ and aspartic acid at position 173 of the CH1;
arginine at position 178 of the CLλ and glutamic acid at position 173 of the CH1;
arginine at position 178 of the CLλ and serine at position 173 of the CH1;
arginine at position 178 of the CLλ and threonine at position 173 of the CH1;
lysine at position 178 of the CLλ and aspartic acid at position 173 of the CH1;
lysine at position 178 of the CLλ and glutamic acid at position 173 of the CH1;
lysine at position 178 of the CLλ and serine at position 173 of the CH1; and
lysine at position 178 of the CLλ and threonine at position 173 of the CH1.

In some aspects, an antigen and/or T cell binding arm comprising a lambda charge pair comprises more than one lambda charge pair. For example, a first antigen and/or T cell binding arm may comprise two, three, four, five, six, seven, eight or nine lambda charge pairs at positions (i) to (ix) described above.

In some aspects, a first antigen and/or T cell binding arm comprises a kappa charge pair and a second antigen binding arm and/or second T cell binding arm both comprise lambda charge pairs, wherein the charged amino acid residues located on a second antigen binding CH1 domain and CLλ are the opposite charge to those located on a second T cell binding CH1 domain and CLλ.

In some exemplary aspects, a positively charged amino acid residue in the lambda charge pair is located on a light chain and the negatively charged amino acid residue in the lambda charge pair is located on a heavy chain. In some aspects, a negatively charged amino acid residue is located on a light chain and a positively charged amino acid residue in the lambda charge pair is located on a heavy chain.

### Kappa Charge Pairs

In the T cell engaging molecules described herein, at least one antigen and/or T cell binding arm comprises a kappa charge pair. As described above, kappa charge pairs refer to a positively charged amino acid residue and a negatively charged amino acid residue, one of which is located in a kappa light chain (e.g. CLκ) and the other in a heavy chain (e.g. CH1) of an antigen and/or T cell binding arm, located at positions intended to promote association of a light chain and CH1 of a second antigen and/or T cell binding arm.

In some aspects, an antigen and/or T cell binding arm(s) containing a CLκ comprises a kappa charge pair located at position 133 in the CLκ and position 183 in the second CH1. In some aspects, a negatively charged amino acid residue in a kappa charge pair is at position 133 of the CLκ and a positively charged amino acid residue in a kappa charge pair 183 of the second CH1. In some aspects, a positively charged amino acid residue in a kappa charge pair is at position 133 of the CLκ and a negatively charged amino acid residue in a kappa charge pair 183 of the second CH1. In some aspects, a negatively charged amino acid residue (e.g. at position 133 of the CLκ) is a glutamic acid, and wherein a positively charged amino acid residue (e.g. at position 183 of the second CH1) is a lysine. As noted, this numbering is according to EU numbering.

In some aspects, a second antigen binding arm and a second T cell binding arm both comprise a kappa charge pair. In some aspects, a first antigen binding arm comprises a kappa charge pair and a second antigen binding arm and second T cell binding arm comprise a lambda charge pair.

In some aspects, a positively charged amino acid residue in a kappa charge pair in a second antigen binding arm is located on a second CH1 and a negatively charged amino acid residue located on a second light chain; and a positively charged amino acid residue in a kappa charge pair in the second T cell binding arm is located on a third light chain and the negatively charged amino acid residue located on a third CH1. In some aspects, a positively charged amino acid residue in a kappa charge pair in a second antigen binding arm is located on a second light chain and a negatively charged amino acid residue located on a second CH1; and a positively charged amino acid residue in a kappa charge pair in a second antigen binding arm is located on a third CH1 and a negatively charged amino acid residue located on the a light chain

In some aspects, a first antigen binding arm comprises a lambda charge pair comprising a positively charged amino acid residue and a negatively charged amino acid residue located at the interface between the first CH1 and the CLλ; a second antigen binding arm comprises a kappa charge pair comprising a positively charged amino acid residue and a negatively charged amino acid residue located at the interface between the second CH1 and the CLκ of the second light chain; and a second T cell binding arm comprises a kappa charge pair comprising a positively charged amino acid residue and a negatively charged amino acid residue located at the interface between the third CH1 and the CLκ of the third light chain, and wherein the charged amino acid residues located on the third CH1 and CLκ of the third light chain are the opposite charge to those located on the second CH1 and the CLκ of the second light chain.

In other aspects, a first antigen binding arm contains a kappa charge pair and a second antigen binding arm and second T cell binding arm contain oppositely charged lambda charge pairs. In some aspects, a first antigen binding arm comprises a kappa charge pair comprising a positively charged amino acid residue and a negatively charged amino acid residue located at the interface between the first CH1 and the CLκ; a second antigen binding arm comprises a lambda charge pair comprising a positively charged amino acid residue and a negatively charged amino acid residue located at the interface between the second CH1 and the CL of the second light chain; and a second T cell binding arm comprises a lambda charge pair comprising a positively charged amino acid residue and a negatively charged amino acid residue located at the interface between a third CH1 and a CLλ of a third light chain, and wherein the charged amino acid residues located on a third CH1 and CLλ of a third light chain are the opposite charge to those located on a second CH1 and the CLλ of the second light chain.

For example, a charge pair in a second antigen binding arm may be formed from a positively charged amino acid residue in a second CH1 and a negatively charged amino acid residue in a second light chain, and a kappa charge pair in a second T cell binding arm may be formed from a negatively charged amino acid residue in a third CH1 and a positively charged amino acid residue in a third light chain.

Alternatively, the charge pair in a second antigen binding arm may be formed from a negatively charged amino acid residue in a second CH1 and a positively charged amino acid residue in a second light chain, and a kappa charge pair in a second T cell binding arm may be formed from a positively charged amino acid residue in a third CH1 and a negatively charged amino acid residue in a third light chain.

As described in the examples, several methods are known that can be used to determine correct light chain paring. These include mass spectrometry-based approaches that can be used to establish association of the correct heavy/light chain. In some aspects, when T cell engaging molecules contain a mixture of kappa and lambda light chains, the ratio of kappa and lambda light chains in the assembled T cell engaging molecule can be determined using microfluidics-based electrophoresis as a readout of the correct light chain ratio.

Accordingly, in some aspects, the T cell engaging molecule containing the lambda charge pair exhibits improved correct light chain pairing when compared to an equivalent T cell engaging molecule that lacks the lambda charge pair. In some aspects, the trivalent antibody containing the lambda charge pair exhibits a correct light chain ratio greater than 90%, 95%, 96%, 97%, 98% or 99% (e.g. as determined using a microfluidics-based electrophoresis method), optionally after the T cell engaging molecule has been purified using light chain affinity purification.

As described herein, techniques such as light chain affinity chromatography that utilizes affinity resins specific for either CLκ or CL can be used to selectively purify T cell engaging molecules based on their light chain. Examples of such affinity resins include the LambdaFabSelect and KappaSelect resins available from GE Healthcare. Such methods can be used to selectively purify T cell engaging molecules containing both CLκ and CL and can therefore be used to improve production of T cell engaging molecules.

### V. Engineered Disulfides

In some aspects, the T cell engaging molecules contain engineered disulfides in addition to the charge pairs. By “engineered disulfides” it is meant that a native inter-chain disulfide bond at the CH1-CL interface (e.g. at 220 of the CH1 and 212 of the LC) of at least one of a first antigen binding arm, a second antigen binding arm, a first T cell binding arm, or a second T cell binding arm has been replaced by an engineered (non-native) interchain disulfide, while the other binding arm or arms contain the native interchain disulfide bond at the CH1-CL interface. An engineered disulfide is typically formed by engineering cysteines into the CL of a light chain and the CH1 domain of the corresponding heavy chain and replacing the cysteines that normally form the interchain disulfide. Disclosure related to the introduction of engineered disulfide into antibodies for the purpose of promoting heterodimerization can found e.g., in U.S. Pat. No. 9,527,927 and Mazor, 2015, which are herein incorporated by reference in their entirety.

The formation of disulfide bonds between cysteine residues occurs during the folding of many proteins that enter the secretory pathway. As the polypeptide chain collapses, cysteines brought into proximity can form covalent linkages during a process catalyzed by members of the protein disulfide isomerase family. The term “disulfide link” or “disulfide linked” as used herein, refers to the single covalent bond formed from the coupling of thiol groups, especially of cysteine residues. In some aspects, the covalent linkage between two cysteines is between the two sulfur atoms of each residue. However, depending on the environment, not all protein species may have a disulfide present at all times, for example, in the event of disulfide reduction. Thus, the term “disulfide link” or “disulfide linked” (whether native or engineered), in some aspects, also refers to the presence of two cysteine residues that are capable of forming a disulfide link, irrespective of whether or not they are actually linked at that individual point in time.

In some aspects, a disulfide link between a light chain and CH1 in at least one of the antigen and/or T cell binding arms is capable of being formed between a pair of cysteines engineered into the light chain and CH1 domain of that antigen and/or T cell binding arm. In aspects, a disulfide link between a light chain and CH1 in two of the binding arms (e.g. a first and a second antigen binding arm, a first and second T cell binding arms, a first antigen binding arm and a first T cell binding arm, or a second antigen binding arm and a second T cell binding arm) is capable of being formed between a pair of cysteines engineered into the light chain and CH1 of those two binding arms.

In some aspects, a disulfide link between a first light chain and a CH1 is capable of being formed between a pair of cysteines engineered into the first light chain and the first CH1. In some aspects, a disulfide link between a third light chain and third CH1 is capable of being formed between a pair of cysteines engineered into the third light chain and the third CH1. As described above, the light chains may comprise a CLλ or a CLκ. In some aspects, a pair of cysteines engineered into the CLλ and CH1 are located at position 122 of the CLλ and position 126 of the CH1, and wherein the same CLλ comprises a non-cysteine residue at position 212 and the same CH1 comprises a non-cysteine residue at position 220. In some aspects, the non-cysteine residues are valines.

In some aspects, a pair of cysteines engineered into a constant light chain kappa region (CLκ) and CH1 are located at position 121 of the CLκ and position 126 of the CH1, and wherein the same CLκ comprises a non-cysteine residue at position 214 and the same CH1 comprises a non-cysteine residue at position 220. In some embodiments, the non-cysteine residues are valines.

In some aspects, a disulfide link between a first light chain and first CH1 is capable of being formed between a pair of cysteines engineered into the first light chain and first CH1, a disulfide link formed between a second light chain and second CH1 is formed between a pair of native cysteines, and a disulfide link formed between a third light chain and third CH1 is either: formed between a pair of native cysteines; or capable of being formed between a pair of cysteines engineered into the third light chain polypeptide and the third heavy chain polypeptide, wherein the pair of cysteines inserted into the third light and heavy chain polypeptides are at different amino acid residue positions to the pair of cysteines inserted into the first light and heavy chain polypeptides.

In some aspects, a disulfide link between a first light chain and first CH1 is capable of being formed between a pair of cysteines engineered into position 122 of the CLλ and position 126 of the first CH1, wherein the CLλ comprises a non-cysteine residue at position 212 and the first CH1 comprises a non-cysteine residue at position 220; a disulfide link formed between a second light chain and second CH1 is formed between a pair of native cysteines; and a disulfide link capable of being formed between a third light chain and third CH1 is capable of being formed between a pair of cysteines engineered into position 121 of the CLκ and position 126 of the first CH1, wherein the CLκ comprises a non-cysteine residue at position 214 and the first CH1 comprises a non-cysteine residue at position 220.

In some aspects, a T cell engaging molecule comprises a first antigen binding arm with a lambda charge pair as described above and an engineered disulfide, a second antigen binding arm with a kappa charge pair as described above and a native disulfide, and a first T cell binding arm with a kappa charge pair as described above and an engineered disulfide.

In some aspects, a T cell engaging molecule comprises other combinations of charge pairs and engineered disulfides. In some aspects, a first antigen binding arm comprises a lambda charge pair and native disulfides, and a first and second T cell binding arms comprise a kappa charge pair and engineered disulfides. In some aspects, a first T cell binding arm comprises a kappa charge pair and engineered disulfides, and a first and second antigen binding arms comprise the lambda charge pair and native disulfides. In some aspects, a second antigen binding arm comprises a kappa charge pair and native disulfides, and a first and second T cell binding arm comprise the lambda charge pairs and engineered disulfides.

### VI. Fc Region Modifications

In some aspects, a first and second antigen binding arm further comprise a first and second Fc region (i.e. further comprising the CH2 and CH3 regions of a heavy chain).

In some aspects, a T cell engaging molecule comprises one or more modifications in one or more of the CH1, CH2 and CH3 domains that promote formation of a multivalent T cell engaging molecule by facilitating pairing of the first and second Fc regions. In some aspects, a T cell engaging molecule comprises a Knobs into Holes (KiH) Fc modification based on single amino acid substitutions in the CH3 domains that promote heavy chain heterodimerization as described in Ridgway, 1996. The knob variant heavy chain CH3 has a small amino acid has been replaced with a larger one, thereby generating a protuberance (knob) on the surface of said CH3 domain, and the hole variant has a large amino acid has replaced with a smaller one thereby generating a cavity (hole) on the surface of said CH3 domain. Additional modifications may also be introduced to stabilize the association between the heavy chains.

In some aspects, CH3 modifications to enhance heterodimerization include, for example, “hole” mutations Y407V/T366S/L368A on one Fc region and “knob” mutation T366W on the other Fc region. In some aspects, a T cell engaging molecule further include a stabilizing cystine mutations Y349C (e.g. on the Fc region with the “hole” mutation) and stabilizing S354C mutation on the other Fc region (e.g. on the Fc region with the “knob” mutation”.

In some aspects, a substitution to generate a knob is a substitution to tryptophan at position 366 and a substitution to generate a hole is one or more of the following:
iv) a substitution to valine at position 407;
ii) a substitution to serine at position 366; and
iii) a substitution to alanine at position 368.

In some aspects, a T cell engaging molecule comprises a “knob” present on a first antigen binding arm containing a lambda charge pair and a hole on a second antigen binding arm containing one of the kappa charge pairs. However, the opposite arrangement is also specifically contemplated, i.e. where the “hole” is present on a CH3 of a first antigen and/or T cell binding arm and the “knob” is present on a CH3 of a second antigen and/or T cell binding arm.

Other examples of CH3 modification to enhance heterodimerization are described in, e.g. Table 1 of Brinkmann and Kontermann, 2017 MABS 9(2), 182-212, which is herein specifically incorporated by reference.

For example, the one Fc region may include a modification to allow fractionated elution by protein A chromatography as described in Tustian, 2016. Briefly, one of the Fc regions may comprise a modification that ablates binding to protein A (termed Fc*), allowing for selective purification of a heterodimeric FcFc* multivalent product. Examples of suitable modifications for generating an Fc* region include substitution of H435 with arginine and Y436 with phenylalanine.

Other Fc modifications that can be used in addition to those used for enhancing heterodimerization are those that reduce or abrogate binding of a T cell engaging molecule to one or more Fcγ receptors, such as FcγRI, FcγRIIa, FcγRIIb, FcγRIII and/or to complement. Such mutations reduce or abrogate Fc effector functions. Mutations for reduce or abrogate binding of antibody molecule to one or more Fcγ receptors and complement are known and include the “triple mutation” or “TM” of L234F/L235E/P331S described for example in Organesyan, 2008.

In some aspects, a first antigen binding arm comprises a lambda charge pair and a first Fc region, a disulfide link between a first light chain and first CH1 is capable of being formed between a pair of cysteines engineered into the first light chain and first CH1, and the first Fc region comprises a “knob” mutation; a second antigen binding arm comprises a kappa charge pair and a second Fc region, a disulfide link formed between the second light chain and second CH1 is formed between a pair of native cysteines, and the second Fc region comprises a “hole” mutation; and a T cell binding arm comprises a kappa charge pair, wherein a charged amino acid residues located on a third CH1 and CLκ of a third light chain are the opposite charge to those located on the second CH1 and the CLκ of the second light chain, and wherein the disulfide link formed between the third light chain and third CH1 is either: formed between a pair of native cysteines; or capable of being formed between a pair of cysteines engineered into the third light chain polypeptide and the third heavy chain polypeptide, wherein the pair of cysteines inserted into the third light and heavy chain polypeptides are at different amino acid residue positions to the pair of cysteines inserted into the first light and heavy chain polypeptides.

Non-limiting examples of multivalent T cell engaging molecules comprising a lambda charge pair, a kappa charge pair, engineered disulfides and modifications to facilitate heterodimerization of the first and second Fc regions are provided in the examples.

In some aspects, T cell engaging molecules comprise amino acid modifications. In some aspects, the modification is a substitution of an amino acid residue to any other naturally occurring or non-naturally occurring amino acid residue.

Naturally occurring residues may be divided into classes based on common side chain properties:
1) nonpolar, aliphatic: glycine (G), methionine (M), alanine (A), valine (V), leucine (L), isoleucine (I);
2) polar: cysteine I, asparagine (N), glutamine (Q), proline (P);
3) polar, partially negatively charged: serine (S), threonine (T);
4) acidic (negatively charged): aspartic acid (D), glutamic acI (E);
5) basic (positively charged): histidine (H), lysine (K), arginine I;
6) aromatic: tryptophan (W), tyrosine (Y), phenylalanine (F).

As described above, serine (S) and threonine (T) have an isoelectric point below 6 and are partially negatively charged at neutral pH, hence they are classed here as “polar, partially negatively charged.”

The amino acid substitution may be a conservative amino acid substitution. Conservative amino acid substitutions may involve exchange of a member of one of these classes with another member of the same class. For example, a conservative amino acid substitution may be a substitution of the acidic amino acid glutamic acid (E) for the acidic amino acid aspartic acid (D).

In one aspect, the antibody or antigen-binding fragment thereof does not have one or more effector functions. For instance, in one aspect, the antibody or antigen-binding fragment thereof has no antibody-dependent cellular cytotoxicity (ADCC) activity and/or no complement-dependent cytotoxicity (CDC) activity. In one aspect, the antibody or antigen-binding fragment thereof does not bind to an Fc receptor and/or complement factors. In one aspect, the antibody or antigen-binding fragment thereof has no effector function. In one aspect, the antibody or antigen-binding fragment comprises an Fc region comprising a triple mutation (TM) which has reduced antibody dependent cellular cytotoxicity (ADCC) compared to an antibody having a wild type Fc region. In one aspect, the antibody or antigen binding fragment thereof has an Fc region which comprises a L234F/L235E/P331S triple mutation (TM). In one aspect, the antibody or antigen-binding fragment thereof has no effector function or. In one aspect, the antibody or antigen binding fragment thereof has an Fc region which comprises E233P/L234V/L235A/G236del/S267K. In one aspect, E233P/L234V/L235A/G236del/S267K is referred to as “null” or “Fc null”.

### VII. T Cell Engaging Molecules

The present disclosure provides T cell engaging molecules. T cell engaging molecules as described herein are capable of binding two different epitopes, either on the same or, in, di some aspects, different antigens. In some aspects, the T cell engaging molecules comprise at least one antigen binding arm and at least one T cell binding arm. According to the present disclosure, an “antigen binding arm” comprises a heavy chain comprising a VH domain and CH1 domain and a light chain comprising a VL domain and a light chain constant domain, where the light chain constant domain is disulfide linked to the CH1 domain, and wherein the antigen binding arm binds an antigen of interest. In some aspects, a “T cell binding arm” comprises a heavy chain comprising a VH domain and CH1 domain and a light chain comprising a VL domain and a light chain constant domain, where the light chain constant domain is disulfide linked to the CH1 domain, and wherein the T cell binding arm binds a T cell antigen.

In some aspects, an antigen binding arm and/or a T cell binding arm in the T cell engaging molecule further comprise an Fc region. In some aspects, an antigen binding arm and/or a T cell binding arm in the T cell engaging molecule comprise complete heavy chains (i.e. a VH domain, CH1 domain, hinge region, CH2 domain and CH3 domain).

In some aspects, a T cell engaging molecule comprises (i) a first antigen binding arm comprising a heavy chain comprising a VH and a heavy chain constant region comprising a CH1, CH2, and CH3 domain, and a light chain comprising a VL and a light chain constant domain; (ii) a T cell binding arm comprising a heavy chain comprising a VH and a heavy chain constant region comprising a CH1, CH2, and CH3 domain, and a light chain comprising a VL and a light chain constant domain; and an Fc region, wherein the heavy chain of the antigen binding arm and the heavy chain of the T cell binding arm are attached to the Fc domain. An exemplary T cell engaging molecule is shown in FIG. 1A.

In some aspects, a T cell engaging molecule comprises (i) a first antigen binding arm comprising a heavy chain comprising a VH and a heavy chain constant region comprising a CH1, CH2, and CH3 domain, and a light chain comprising a VL and a light chain constant domain; (ii) a T cell binding arm comprising a heavy chain comprising a VH and a heavy chain constant region comprising a CH1, CH2, and CH3 domain, and a light chain comprising a VL and a light chain constant domain; and (iii) a second antigen binding arm comprising a heavy chain comprising a VH and a CH1 domain and a light chain comprising a VL and a light chain constant domain; wherein the heavy chain of the second antigen binding arm is attached to the heavy chain of the T cell binding arm through a peptide linker. Exemplary T cell engaging molecules are shown in FIGS. 1A-1D.

In some aspects, a light chain of an antigen binding arm is disulfide linked to a heavy chain of the antigen binding arm in the T cell engaging molecule via a native inter-chain disulfide bond (e.g., a disulfide bond present in an IgG antibody). In some aspects, a light chain of an antigen binding arm is disulfide linked to a heavy chain of the antigen binding arm in the T cell engaging molecule via an engineered disulfide bond. In some aspects, a light chain of a T cell binding arm is disulfide linked to a heavy chain of the T cell binding arm in the T cell engaging molecule via a native inter-chain disulfide bond. In some aspects, a light chain of a T cell binding arm is disulfide linked to a heavy chain of the T cell binding arm in the T cell engaging molecule via an engineered disulfide bond.

In some aspects, a heavy chain of an antigen binding arm is attached to a heavy chain of another antigen binding arm via a peptide linker. In some aspects, a heavy chain of an antigen binding arm is attached to a T cell binding arm via a peptide linker. In some aspects, a peptide linker consists of 5 to 100 amino acids, 5 to 50 amino acids, 5 to 25 amino acids, or 5 to 15 amino acids. In some aspects, a peptide linker is formed mainly from glycine and serine amino acid residues and in some aspects comprises the amino acid sequences GGGGS or SGGGGS. In some aspects, the peptide linker comprises or consists of SEQ ID NO: 89. In some aspects, the linker comprises from 1 to about 10 copies of SEQ ID NO: 89. In some aspects, the linker comprises 2 copies of SEQ ID NO: 89.

In some aspects, a T cell engaging molecule comprises (a) at least one antigen binding arm that binds STEAP2 and comprises a heavy chain comprising a heavy chain variable domain (VH) and a heavy chain constant domain (CH1); and a light chain comprising a light chain variable domain (VL) and a light chain constant domain (CL); (b) a T cell binding arm comprising a heavy chain comprising a VH domain and a CH1 domain; a light chain comprising a VL domain and a light chain constant domain; and (c) an Fc domain comprising a first Fc region and a second Fc region, each Fc region comprising a CH2 domain and a CH3 domain, said Fc domain further comprising at least one modification to promote heterodimerization.

In some aspects, a T cell engaging molecule comprises an antigen binding arm that binds STEAP2 and comprises a heavy chain comprising a VH domain comprising a VH-CDR1 selected from SEQ ID Nos: 1, 9, 17, 103, 111, 127, 135, and 143; a VH-CDR2 selected from SEQ ID NO: 2, 10, 18, 104, and 112; a VH-CDR3 selected from SEQ ID NO: 3, 11, 19, 94, 96, 98, 105, 113, 129, 137, and 145; and a CH1 domain. In some aspects, the antigen binding arm of the T cell engaging molecule further comprises a light chain comprising a VL domain comprising a VL-CDR1 selected from SEQ ID Nos: 4, 12, 20, 100, 108, 130, 138, and 146; a VL-CDR2 selected from SEQ ID NO: 5, 13, 21, 101, 109, 131, 139, and 147; a VL-CDR3 selected from SEQ ID Nos: 6, 14, 22, 102, 110, 132, 140, and 148; and a light chain constant domain.

In some aspects, the T cell engaging molecule further comprises a T cell binding arm that binds CD3 and comprises a heavy chain comprising a VH domain comprising a VH-CDR1 selected from SEQ ID Nos: 36, 40, and 44; a VH-CDR2 selected from SEQ ID NO: 37, 41, and 45; a VH-CDR3 selected from SEQ ID NO: 38, 42, and 46; and a CH1 domain. In some aspects, the T cell binding arm further comprises a light chain comprising a VL comprising a VL-CDR1 selected from SEQ ID Nos: 27 and 31; a VL-CDR2 selected from SEQ ID Nos: 28 and 32; a VL-CDR3 selected from SEQ ID Nos: 29 and 33; and a light chain constant domain. In some aspects, the T cell engaging molecule further comprises an Fc domain comprising a first Fc region and a second Fc region, each Fc region comprising a CH2 domain and a CH3 domain; further comprising at least one modification to promote heterodimerization.

In some aspects, the T cell engaging molecule further comprises a second antigen binding arm that binds STEAP2 and comprises a heavy chain comprising a VH domain comprising a VH-CDR1 selected from SEQ ID Nos: 1, 9, 17, 103, 111, 127, 135, and 143; a VH-CDR2 selected from SEQ ID Nos: 2, 10, 18, 104, 112, 128, 136, and 144; a VH-CDR3 selected from SEQ ID Nos: 3, 11, 19, 94, 96, 98, 105, 113, 129, 137, and 145; and a CH1 domain. In some aspects, the second antigen binding arm of the T cell engaging molecule further comprises a light chain comprising a VL comprising a VL-CDR1 selected from SEQ ID Nos: 4, 12, 20, 100, 108, 130, 138, and 146; a VL-CDR2 selected from SEQ ID NO: 5, 13, 21, 101, 109, 131, 139, and 147; a VL-CDR3 selected from SEQ ID Nos: 6, 14, 22, 102, 110, 132, 140, and 148; and a light chain constant domain.

In some aspects, one of the CH3 domains of the Fc region of the T cell engaging molecule comprises a knob mutation, and the other CH3 domain comprises a hole mutation.

In some aspects, a CH1 domain and a light chain constant domain of one or more of each of an antigen binding arm and a T cell binding arm of the T cell engaging molecule further comprise a charge pair substitution comprising a first charged amino acid substitution in the CH1 and a second charged amino acid substitution in the light chain constant domain, wherein the first charged amino acid substitution and the second charged amino acid substitution have an opposite charge.

In some aspects, a light chain constant domain of one or more of each of an antigen binding arm and a T cell binding arm is a lambda light chain constant domain (CLλ) and the charge pair is a lambda charge pair comprising a positively charged amino acid residue selected from arginine, lysine or histidine, and a negatively charged amino acid residue selected from aspartic acid, glutamic acid, serine or threonine. In some aspects, the charged amino acids of a lambda charge pair are located at one or more of the following positions:
(i) position 117 in the CLλ and position 141 in the CH1 domain;
(ii) position 117 in the CLλ and position 185 in the CH1 domain;
(iii) position 119 in the CLλ and position 128 in the CH1 domain;
(iv) position 134 in the CLλ and position 128 in the CH1 domain;
(v) position 134 in the CLλ and position 145 in the CH1 domain;
(vi) position 134 in the CLλ and position 183 in the CH1 domain;
(vii) position 136 in the CLλ and position 185 in the CH1 domain;
(viii) position 178 in the CLλ and position 173 in the CH1 domain; and/or
(ix) position 117 in the CLλ and position 187 in the CH1 domain, wherein the numbering is according to the EU index.

In some aspects, a light chain constant domain of one or more of each of an antigen binding arm and a T cell binding arm is a lambda light chain constant domain (CLλ) and the charge pair is a lambda charge pair, wherein:
(c) the charged amino acid at position 117 is arginine and the charged amino acid at position 141 is aspartic acid; the charged amino acid at position 117 is arginine and the charged amino acid at position 141 is glutamic acid; the charged amino acid at position 117 is arginine and the charged amino acid at position 141 is serine; the charged amino acid at position 117 is arginine and the charged amino acid at position 141 is threonine; the charged amino acid at position 117 is lysine and the charged amino acid at position 141 is aspartic acid; the charged amino acid at position 117 is lysine and the charged amino acid at position 141 is glutamic acid; the charged amino acid at position 117 is lysine and the charged amino acid at position 141 is serine; or the charged amino acid at position 117 is lysine and the charged amino acid at position 141 is threonine;
(ii) the charged amino acid at position 117 is arginine and the charged amino acid at position 185 is aspartic acid; the charged amino acid at position 117 is arginine and the charged amino acid at position 185 is glutamic acid; the charged amino acid at position 117 is arginine and the charged amino acid at position 185 is serine; the charged amino acid at position 117 is arginine and the charged amino acid at position 185 is threonine; the charged amino acid at position 117 is lysine and the charged amino acid at position 185 is aspartic acid; the charged amino acid at position 117 is lysine and the charged amino acid at position 185 is glutamic acid; the charged amino acid at position 117 is lysine and the charged amino acid at position 185 is serine; or the charged amino acid at position 117 is lysine and the charged amino acid at position 185 is threonine;
(iii) the charged amino acid at position 119 is arginine and the charged amino acid at position 128 is aspartic acid; the charged amino acid at position 119 is arginine and the charged amino acid at position 128 is glutamic acid; the charged amino acid at position 119 is arginine and the charged amino acid at position 128 is serine; the charged amino acid at position 119 is arginine and the charged amino acid at position 128 is threonine; the charged amino acid at position 119 is lysine and the charged amino acid at position 128 is aspartic acid; the charged amino acid at position 119 is lysine and the charged amino acid at position 128 is glutamic acid; the charged amino acid at position 119 is lysine and the charged amino acid at position 128 is serine; or the charged amino acid at position 119 is lysine and the charged amino acid at position 128 is threonine;
(iv) the charged amino acid at position 134 is arginine and the charged amino acid at position 128 is aspartic acid; the charged amino acid at position 134 is arginine and the charged amino acid at position 128 is glutamic acid; the charged amino acid at position 134 is arginine and the charged amino acid at position 128 is serine; the charged amino acid at position 134 is arginine and the charged amino acid at position 128 is threonine; the charged amino acid at position 134 is lysine and the charged amino acid at position 128 is aspartic acid; the charged amino acid at position 134 is lysine and the charged amino acid at position 128 is glutamic acid; the charged amino acid at position 134 is lysine and the charged amino acid at position 128 is serine; or the charged amino acid at position 134 is lysine and the charged amino acid at position 128 is threonine;
(v) the charged amino acid at position 134 is arginine and the charged amino acid at position 145 is aspartic acid; the charged amino acid at position 134 is arginine and the charged amino acid at position 145 is glutamic acid; the charged amino acid at position 134 is arginine and the charged amino acid at position 145 is serine; the charged amino acid at position 134 is arginine and the charged amino acid at position 145 is threonine; the charged amino acid at position 134 is lysine and the charged amino acid at position 145 is aspartic acid; the charged amino acid at position 134 is lysine and the charged amino acid at position 145 is glutamic acid; the charged amino acid at position 134 is lysine and the charged amino acid at position 145 is serine; or the charged amino acid at position 134 is lysine and the charged amino acid at position 145 is threonine;
(vi) the charged amino acid at position 134 is arginine and the charged amino acid at position 183 is aspartic acid; the charged amino acid at position 134 is arginine and the charged amino acid at position 183 is glutamic acid; the charged amino acid at position 134 is arginine and the charged amino acid at position 183 is serine; the charged amino acid at position 134 is arginine and the charged amino acid at position 183 is threonine; the charged amino acid at position 134 is lysine and the charged amino acid at position 183 is aspartic acid; the charged amino acid at position 134 is lysine and the charged amino acid at position 183 is glutamic acid; the charged amino acid at position 134 is lysine and the charged amino acid at position 183 is serine; or the charged amino acid at position 134 is lysine and the charged amino acid at position 183 is threonine;
(vii) the charged amino acid at position 136 is arginine and the charged amino acid at position 185 is aspartic acid; the charged amino acid at position 136 is arginine and the charged amino acid at position 185 is glutamic acid; the charged amino acid at position 136 is arginine and the charged amino acid at position 185 is serine; the charged amino acid at position 136 is arginine and the charged amino acid at position 185 is threonine; the charged amino acid at position 136 is lysine and the charged amino acid at position 185 is aspartic acid; the charged amino acid at position 136 is lysine and the charged amino acid at position 185 is glutamic acid; the charged amino acid at position 136 is lysine and the charged amino acid at position 185 is serine; or the charged amino acid at position 136 is lysine and the charged amino acid at position 185 is threonine;
(viii) the charged amino acid at position 178 is arginine and the charged amino acid at position 173 is aspartic acid; the charged amino acid at position 178 is arginine and the charged amino acid at position 173 is glutamic acid; the charged amino acid at position 178 is arginine and the charged amino acid at position 173 is serine; the charged amino acid at position 178 is arginine and the charged amino acid at position 173 is threonine; the charged amino acid at position 178 is lysine and the charged amino acid at position 173 is aspartic acid; the charged amino acid at position 178 is lysine and the charged amino acid at position 173 is glutamic acid; the charged amino acid at position 178 is lysine and the charged amino acid at position 173 is serine; or the charged amino acid at position 178 is lysine and the charged amino acid at position 173 is threonine; and/or
(ix) the charged amino acid at position 117 is arginine and the charged amino acid at position 187 is aspartic acid; the charged amino acid at position 117 is arginine and the charged amino acid at position 187 is glutamic acid; the charged amino acid at position 117 is arginine and the charged amino acid at position 187 is serine; the charged amino acid at position 117 is arginine and the charged amino acid at position 187 is threonine; the charged amino acid at position 117 is lysine and the charged amino acid at position 187 is aspartic acid; the charged amino acid at position 117 is lysine and the charged amino acid at position 187 is glutamic acid; the charged amino acid at position 117 is lysine and the charged amino acid at position 187 is serine; or the charged amino acid at position 117 is lysine and the charged amino acid at position 187 is threonine.

In some aspects, a light chain constant domain of one or more of each an antigen binding arm and a T cell binding arm of the T engaging molecule is a kappa light chain constant domain (CLκ) and the charge pair is a kappa charge pair comprising a positively charged amino acid residue selected from arginine, lysine or histidine, and a negatively charged amino acid residue selected from aspartic acid, glutamic acid, serine or threonine, wherein the kappa charge pair is located at position 133 in the CLκ and position 183 in the CH1.

In some aspects, a light chain constant domain of one or more of each an antigen binding arm and a T cell binding arm of the T engaging molecule is a kappa light chain constant domain (CLκ) and the charge pair is a kappa charge pair wherein the charged amino acid at position 133 is glutamic acid and the charged amino acid at position 183 is lysine; or the charged amino acid at position 133 is lysine and the charged amino acid at position 183 is glutamic acid.

In some aspects, a light chain constant domain of a T cell binding arm is a CLλ and the charge pair is a lambda charge pair as described herein and the light chain constant domain of a first and/or second antigen binding domain is a lambda or a kappa charge pair and the first antigen binding arm comprises a charged amino acid in the CH1 that is of a same charge as a charged amino acid in the CH1 domain of the second antigen binding arm.

In some aspects, a light chain constant domain of a T cell binding arm is a CLλ and the charge pair is a lambda charge pair as described herein and the light chain constant domain of a first and a second antigen binding arm is a kappa charge pair and a charged amino acid in the CH1 domain of the first antigen binding arm is of a same charge as a charged amino acid in the CH1 domain of the second antigen binding arm.

In some aspects, a CH1 domain of an antigen binding arm or a T cell binding arm is capable of being linked to a light chain constant domain through an engineered disulfide link.

In some aspects, a CH1 domain that is capable of being linked to the light chain constant domain through an engineered disulfide link comprises (i) a substitution of a native cysteine to a non-cysteine amino acid and (ii) a substitution of a native non-cysteine amino acid to a cysteine; and the light chain constant domain comprises (i) a substitution of a native cysteine to a non-cysteine amino acid and (ii) a substitution of a native non-cysteine amino acid to a cysteine; wherein the substituted cysteine of the light chain constant domain and the substituted cysteine of the CH1 domain can form a disulfide bond.

In some aspects, a CH1 domain that is capable of being linked to the light chain constant domain through an engineered disulfide link comprises (i) a substitution of a native cysteine to a non-cysteine amino acid at position 220, and (ii) a substitution of a native non-cysteine amino acid to a cysteine at position 126; and the light chain constant domain comprises (i) a substitution of a native cysteine to a non-cysteine amino acid at position 212, and (ii) a substitution of a native non-cysteine amino acid to a cysteine at position 122, wherein the cysteine at position 126 of the CH1 domain and the cysteine at position 122 of the light chain constant domain can form a disulfide bond; wherein the numbering is according to the EU index.

In some aspects, a CH1 domain that is capable of being linked to the light chain constant domain through an engineered disulfide link comprises (i) a substitution of a native cysteine to a non-cysteine amino acid at position 220, and (ii) a substitution of a native non-cysteine amino acid to a cysteine at position 126; and the light chain constant domain comprises (i) a substitution of a native cysteine to a non-cysteine amino acid at position 214, and (ii) a substitution of a native non-cysteine amino acid to a cysteine at position 121, wherein the cysteine at position 126 of the CH1 domain and the cysteine at position 121 of the light chain constant domain can form a disulfide bond; wherein the numbering is according to the EU index.

In some aspects, an antigen binding arm of a T cell engaging molecule comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149.

In some aspects, an antigen binding arm of a T cell engaging molecule comprises a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 90.

In some aspects, an antigen binding arm of a T cell engaging molecule comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149 and a CH1 domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 90.

In some aspects, an antigen binding arm of the T cell engaging molecule comprises a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 8, 16, 24, 107, 115, 134, 142, and 150.

In some aspects, an antigen binding arm of a T cell engaging molecule comprises a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 63.

In some aspects, an antigen binding arm of a T cell engaging molecule comprises a light chain comprising a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 8, 16, 24, 107, 115, 134, 142, and 150 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 63.

In some aspects, an antigen binding arm of a T cell engaging molecule comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149.

In some aspects, an antigen binding arm of a T cell engaging molecule comprises a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 88.

In some aspects, an antigen binding arm of a T cell engaging molecule comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149 and a CH1 domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 88.

In some aspects, a T cell binding arm of a T cell engaging molecule comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 39, 43, and 47.

In some aspects, a T cell binding arm of a T cell engaging molecule comprises a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 79.

In some aspects, a T cell binding arm of a T cell engaging molecule comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 39, 43, and 47 and a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 79.

In some aspects, a T cell binding arm of the T cell engaging molecule comprises a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 30 and 34.

In some aspects, a T cell binding arm of a T cell engaging molecule comprises a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 61.

In some aspects, a T cell binding arm of a T cell engaging molecule comprises a light chain comprising a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 30 and 34 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 61.

In some aspects, a T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 25. In some aspects, a T cell engaging molecule comprises a light chain comprising the amino acid sequence set forth in SEQ ID NO: 25.

In some aspects, a T cell engaging molecule comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 116. In some aspects, a T cell engaging molecule comprises a light chain comprising the amino acid sequence set forth in SEQ ID NO: 116.

In some aspects, a T cell engaging molecule comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117. In some aspects, a T cell engaging molecule comprises a light chain comprising the amino acid sequence set forth in SEQ ID NO: 117.

In some aspects, a T cell engaging molecule comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 118. In some aspects, a T cell engaging molecule comprises a light chain comprising the amino acid sequence set forth in SEQ ID NO: 118.

In some aspects, a T cell engaging molecule comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 119. In some aspects, a T cell engaging molecule comprises a light chain comprising the amino acid sequence set forth in SEQ ID NO: 119.

In some aspects, a T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 35. In some aspects, a T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 81. In some aspects, a T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 88.

In some aspects, a T cell engaging molecule comprises an amino acid sequence set forth in SEQ ID NO: 25. In some aspects, a T cell engaging molecule comprises an amino acid sequence set forth in SEQ ID NO: 35. In some aspects, a T cell engaging molecule comprises an amino acid sequence set forth in SEQ ID NO: 81. In some aspects, a T cell engaging molecule comprises an amino acid sequence set forth in SEQ ID NO: 88.

In some aspects, a T cell engaging molecule comprises the amino acid sequences set forth in SEQ ID NO: 25, 26, 35, 81 and 92.

### VIII. Trivalent Trispecific T Cell Engaging Molecules

The present disclosure provides trivalent trispecific T cell engaging molecules. Trivalent T cell engaging molecules as described herein are capable of binding three different epitopes, either on the same or, in some aspects, different antigens. In some aspects, the trivalent T cell binding molecules comprise at least one antigen binding arm and at least two different T cell binding arms. In some aspects, the antigen binding arm is referred to herein as a “first antigen binding arm”, and the T cell binding arms are referred herein as a “first T cell binding arm” and a “second T cell binding arm”. According to the present disclosure, an “antigen binding arm” comprises a heavy chain comprising a VH domain and CH1 domain and a light chain comprising a VL domain and a light chain constant domain, where the light chain constant domain is disulfide linked to the CH1 domain. In some aspects, a “T cell binding arm” comprises a heavy chain comprising a VH domain and CH1 domain and a light chain comprising a VL domain and a light chain constant domain, where the light chain constant domain is disulfide linked to the CH1 domain. In some aspects, a T cell binding arm comprises only a VH domain.

In some aspects, an antigen binding arm and/or at least one T cell binding arm in the trivalent T cell engaging molecule further comprise an Fc region. In some aspects, an antigen binding arm and/or a T cell binding arm in the trivalent T cell engaging molecule comprise complete heavy chains (i.e. a VH domain, CH1 domain, hinge region, CH2 domain and CH3 domain).

In some aspects, a trivalent T cell engaging molecule comprises (i) an antigen binding arm comprising a heavy chain comprising a VH and a heavy chain constant region comprising a CH1, CH2, and CH3 domain, and a light chain comprising a VL and a light chain constant domain; (ii) a first T cell binding arm comprising a heavy chain comprising a VH and a heavy chain constant region comprising a CH1, CH2, and CH3 domain, and a light chain comprising a VL and a light chain constant domain; and (iii) a second T cell binding arm comprising a heavy chain comprising a VH and a CH1 domain and a light chain comprising a VL and a light chain constant domain; wherein the heavy chain of the second T cell binding arm is attached to the heavy chain of the first T cell binding arm through a peptide linker. An exemplary trivalent T cell engaging molecule is shown in FIG. 1C.

In some aspects, a light chain of an antigen binding arm is disulfide linked to a heavy chain of the antigen binding arm in the trivalent T cell engaging molecule via a native inter-chain disulfide bond (e.g., a disulfide bond present in an IgG antibody). In some aspects, a light chain of an antigen binding arm is disulfide linked to a heavy chain of the antigen binding arm in the trivalent T cell engaging molecule via an engineered disulfide bond. In some aspects, a light chain of a first and/or second T cell binding arm is disulfide linked to a heavy chain of the first and/or second T cell binding arm in the trivalen-105-ngaging1 engaging molecule via a native inter-chain disulfide. In some aspects, a light chain of a first and/or second T cell binding arm is disulfide linked to a heavy chain of the first and/or second T cell binding arm in the T cell engaging molecule via an engineered disulfide bond.

In some aspects, a heavy chain of an antigen binding arm is attached to a heavy chain of another antigen binding arm via a peptide linker. In some aspects, a heavy chain of an antigen binding arm is attached to a T cell binding arm via a peptide linker. In some aspects, a peptide linker consists of 5 to 100 amino acids, 5 to 50 amino acids, 5 to 25 amino acids, or 5 to 15 amino acids. In some aspects, a peptide linker is formed mainly from glycine and serine amino acid residues and in some aspects comprises the amino acid sequences GGGGS or SGGGGS. In some aspects, the peptide linker comprises or consists of SEQ ID NO: 89. In some aspects, the linker comprises from 1 to about 10 copies of SEQ ID NO: 89. In some aspects, the linker comprises 2 copies of SEQ ID NO: 89.

In some aspects, a trivalent T cell engaging molecule comprises (a) at least one antigen binding arm that binds STEAP2 and comprises a heavy chain comprising a VH domain and a CH1 domain; and a light chain comprising a VL and a light chain constant domain; (b) at least one T cell binding arm comprising a heavy chain comprising a VH domain and a CH1 domain; a light chain comprising a VL domain and a light chain constant domain; and (c) an Fc domain comprising a first Fc region and a second Fc region, each Fc region comprising a CH2 domain and a CH3 domain; further comprising at least one modification to promote heterodimerization.

In some aspects, a trivalent T cell engaging molecule comprises (i) at least one antigen binding arm that binds an epitope on STEAP2, (ii) at least one T cell binding arm that binds CD3, and (iii) at least one T cell binding arm that binds CD8.

In some aspects, one of the CH3 domains of the Fc region of the trivalent T cell engaging molecule comprises a knob mutation, and the other CH3 domain comprises a hole mutation.

In some aspects, a CH1 domain and a light chain constant domain of one or more of each of an antigen binding arm and a T cell binding arm of the trivalent T cell engaging molecule further comprise a charge pair substitution comprising a first charged amino acid substitution in the CH1 and a second charged amino acid substitution in the light chain constant domain, wherein the first charged amino acid substitution and the second charged amino acid substitution have an opposite charge.

In some aspects, a light chain constant domain of one or more of each of an antigen binding arm and a T cell binding arm of the trivalent T engaging molecule is a lambda light chain constant domain (CLλ) and the charge pair is a lambda charge pair comprising a positively charged amino acid residue selected from arginine, lysine or histidine, and a negatively charged amino acid residue selected from aspartic acid, glutamic acid, serine or threonine. In some aspects, the charged amino acids of a lambda charge pair are located at one or more of the following positions:
(i) position 117 in the CLλ and position 141 in the CH1 domain;
(ii) position 117 in the CLλ and position 185 in the CH1 domain;
(iii) position 119 in the CLλ and position 128 in the CH1 domain;
(iv) position 134 in the CLλ and position 128 in the CH1 domain;
(v) position 134 in the CLλ and position 145 in the CH1 domain;
(vi) position 134 in the CLλ and position 183 in the CH1 domain;
(vii) position 136 in the CLλ and position 185 in the CH1 domain;
(viii) position 178 in the CLλ and position 173 in the CH1 domain; and/or
(ix) position 117 in the CLλ and position 187 in the CH1 domain, wherein the numbering is according to the EU index.

In some aspects, a light chain constant domain of one or more of each of an antigen binding arm and a T cell binding arm of the trivalent T engaging molecule is a lambda light chain constant domain (CLλ) and the charge pair is a lambda charge pair, wherein:
(c) the charged amino acid at position 117 is arginine and the charged amino acid at position 141 is aspartic acid; the charged amino acid at position 117 is arginine and the charged amino acid at position 141 is glutamic acid; the charged amino acid at position 117 is arginine and the charged amino acid at position 141 is serine; the charged amino acid at position 117 is arginine and the charged amino acid at position 141 is threonine; the charged amino acid at position 117 is lysine and the charged amino acid at position 141 is aspartic acid; the charged amino acid at position 117 is lysine and the charged amino acid at position 141 is glutamic acid; the charged amino acid at position 117 is lysine and the charged amino acid at position 141 is serine; or the charged amino acid at position 117 is lysine and the charged amino acid at position 141 is threonine;
(ii) the charged amino acid at position 117 is arginine and the charged amino acid at position 185 is aspartic acid; the charged amino acid at position 117 is arginine and the charged amino acid at position 185 is glutamic acid; the charged amino acid at position 117 is arginine and the charged amino acid at position 185 is serine; the charged amino acid at position 117 is arginine and the charged amino acid at position 185 is threonine; the charged amino acid at position 117 is lysine and the charged amino acid at position 185 is aspartic acid; the charged amino acid at position 117 is lysine and the charged amino acid at position 185 is glutamic acid; the charged amino acid at position 117 is lysine and the charged amino acid at position 185 is serine; or the charged amino acid at position 117 is lysine and the charged amino acid at position 185 is threonine;
(iii) the charged amino acid at position 119 is arginine and the charged amino acid at position 128 is aspartic acid; the charged amino acid at position 119 is arginine and the charged amino acid at position 128 is glutamic acid; the charged amino acid at position 119 is arginine and the charged amino acid at position 128 is serine; the charged amino acid at position 119 is arginine and the charged amino acid at position 128 is threonine; the charged amino acid at position 119 is lysine and the charged amino acid at position 128 is aspartic acid; the charged amino acid at position 119 is lysine and the charged amino acid at position 128 is glutamic acid; the charged amino acid at position 119 is lysine and the charged amino acid at position 128 is serine; or the charged amino acid at position 119 is lysine and the charged amino acid at position 128 is threonine;
(iv) the charged amino acid at position 134 is arginine and the charged amino acid at position 128 is aspartic acid; the charged amino acid at position 134 is arginine and the charged amino acid at position 128 is glutamic acid; the charged amino acid at position 134 is arginine and the charged amino acid at position 128 is serine; the charged amino acid at position 134 is arginine and the charged amino acid at position 128 is threonine; the charged amino acid at position 134 is lysine and the charged amino acid at position 128 is aspartic acid; the charged amino acid at position 134 is lysine and the charged amino acid at position 128 is glutamic acid; the charged amino acid at position 134 is lysine and the charged amino acid at position 128 is serine; or the charged amino acid at position 134 is lysine and the charged amino acid at position 128 is threonine;
(v) the charged amino acid at position 134 is arginine and the charged amino acid at position 145 is aspartic acid; the charged amino acid at position 134 is arginine and the charged amino acid at position 145 is glutamic acid; the charged amino acid at position 134 is arginine and the charged amino acid at position 145 is serine; the charged amino acid at position 134 is arginine and the charged amino acid at position 145 is threonine; the charged amino acid at position 134 is lysine and the charged amino acid at position 145 is aspartic acid; the charged amino acid at position 134 is lysine and the charged amino acid at position 145 is glutamic acid; the charged amino acid at position 134 is lysine and the charged amino acid at position 145 is serine; or the charged amino acid at position 134 is lysine and the charged amino acid at position 145 is threonine;
(vi) the charged amino acid at position 134 is arginine and the charged amino acid at position 183 is aspartic acid; the charged amino acid at position 134 is arginine and the charged amino acid at position 183 is glutamic acid; the charged amino acid at position 134 is arginine and the charged amino acid at position 183 is serine; the charged amino acid at position 134 is arginine and the charged amino acid at position 183 is threonine; the charged amino acid at position 134 is lysine and the charged amino acid at position 183 is aspartic acid; the charged amino acid at position 134 is lysine and the charged amino acid at position 183 is glutamic acid; the charged amino acid at position 134 is lysine and the charged amino acid at position 183 is serine; or the charged amino acid at position 134 is lysine and the charged amino acid at position 183 is threonine;
(vii) the charged amino acid at position 136 is arginine and the charged amino acid at position 185 is aspartic acid; the charged amino acid at position 136 is arginine and the charged amino acid at position 185 is glutamic acid; the charged amino acid at position 136 is arginine and the charged amino acid at position 185 is serine; the charged amino acid at position 136 is arginine and the charged amino acid at position 185 is threonine; the charged amino acid at position 136 is lysine and the charged amino acid at position 185 is aspartic acid; the charged amino acid at position 136 is lysine and the charged amino acid at position 185 is glutamic acid; the charged amino acid at position 136 is lysine and the charged amino acid at position 185 is serine; or the charged amino acid at position 136 is lysine and the charged amino acid at position 185 is threonine;
(viii) the charged amino acid at position 178 is arginine and the charged amino acid at position 173 is aspartic acid; the charged amino acid at position 178 is arginine and the charged amino acid at position 173 is glutamic acid; the charged amino acid at position 178 is arginine and the charged amino acid at position 173 is serine; the charged amino acid at position 178 is arginine and the charged amino acid at position 173 is threonine; the charged amino acid at position 178 is lysine and the charged amino acid at position 173 is aspartic acid; the charged amino acid at position 178 is lysine and the charged amino acid at position 173 is glutamic acid; the charged amino acid at position 178 is lysine and the charged amino acid at position 173 is serine; or the charged amino acid at position 178 is lysine and the charged amino acid at position 173 is threonine; and/or
(ix) the charged amino acid at position 117 is arginine and the charged amino acid at position 187 is aspartic acid; the charged amino acid at position 117 is arginine and the charged amino acid at position 187 is glutamic acid; the charged amino acid at position 117 is arginine and the charged amino acid at position 187 is serine; the charged amino acid at position 117 is arginine and the charged amino acid at position 187 is threonine; the charged amino acid at position 117 is lysine and the charged amino acid at position 187 is aspartic acid; the charged amino acid at position 117 is lysine and the charged amino acid at position 187 is glutamic acid; the charged amino acid at position 117 is lysine and the charged amino acid at position 187 is serine; or the charged amino acid at position 117 is lysine and the charged amino acid at position 187 is threonine.

In some aspects, a light chain constant domain of one or more of each an antigen binding arm and a T cell binding arm of the trivalent T engaging molecule is a kappa light chain constant domain (CLκ) and the charge pair is a kappa charge pair comprising a positively charged amino acid residue selected from arginine, lysine or histidine, and a negatively charged amino acid residue selected from aspartic acid, glutamic acid, serine or threonine, wherein the kappa charge pair is located at position 133 in the CLκ and position 183 in the CH1.

In some aspects, a light chain constant domain of one or more of each an antigen binding arm and a T cell binding arm of the trivalent T engaging molecule is a kappa light chain constant domain (CLκ) and the charge pair is a kappa charge pair wherein the charged amino acid at position 133 is glutamic acid and the charged amino acid at position 183 is lysine; or the charged amino acid at position 133 is lysine and the charged amino acid at position 183 is glutamic acid.

In some aspects, a light chain constant domain of a T cell binding arm of the trivalent T engaging molecule is a CLλ and the charge pair is a lambda charge pair as described herein and the light chain constant domain of an antigen binding arm and a second T cell binding arm of the trivalent T engaging molecule is a lambda or a kappa charge pair and the antigen binding arm comprises a charged amino acid in the CH1 domain that is of a same charge as a charged amino acid in the CH1 domain of the second T cell binding arm.

In some aspects, a light chain constant domain of a T cell binding arm is a CLλ and the charge pair is a lambda charge pair as described herein and the light chain constant domain of an antigen binding domain and a second T cell binding arm is a kappa charge pair and a charged amino acid in the CH1 domain of the antigen binding arm is of a same charge as a charged amino acid in the CH1 domain of the second T cell binding arm.

In some aspects, a CH1 domain of an antigen binding arm or a T cell binding arm of a trivalent T cell engaging molecule is capable of being linked to a light chain constant domain through an engineered disulfide link.

In some aspects, a CH1 domain that is capable of being linked to the light chain constant domain through an engineered disulfide link comprises (i) a substitution of a native cysteine to a non-cysteine amino acid and (ii) a substitution of a native non-cysteine amino acid to a cysteine; and the light chain constant domain comprises (i) a substitution of a native cysteine to a non-cysteine amino acid and (ii) a substitution of a native non-cysteine amino acid to a cysteine; wherein the substituted cysteine of the light chain constant domain and the substituted cysteine of the CH1 domain can form a disulfide bond.

In some aspects, a CH1 domain that is capable of being linked to the light chain constant domain through an engineered disulfide link comprises (i) a substitution of a native cysteine to a non-cysteine amino acid at position 220, and (ii) a substitution of a native non-cysteine amino acid to a cysteine at position 126; and the light chain constant domain comprises (i) a substitution of a native cysteine to a non-cysteine amino acid at position 212, and (ii) a substitution of a native non-cysteine amino acid to a cysteine at position 122, wherein the cysteine at position 126 of the CH1 domain and the cysteine at position 122 of the light chain constant domain can form a disulfide bond; wherein the numbering is according to the EU index.

In some aspects, a CH1 domain that is capable of being linked to the light chain constant domain through an engineered disulfide link comprises (i) a substitution of a native cysteine to a non-cysteine amino acid at position 220, and (ii) a substitution of a native non-cysteine amino acid to a cysteine at position 126; and the light chain constant domain comprises (i) a substitution of a native cysteine to a non-cysteine amino acid at position 214, and (ii) a substitution of a native non-cysteine amino acid to a cysteine at position 121, wherein the cysteine at position 126 of the CH1 domain and the cysteine at position 121 of the light chain constant domain can form a disulfide bond; wherein the numbering is according to the EU index.

In some aspects, a trivalent T cell engaging molecule comprises an antigen binding arm that binds STEAP2 and comprises a heavy chain comprising a VH domain comprising a VH-CDR1 selected from SEQ ID Nos: 1, 9, 17, 103, 111, 127, 135, and 143; a VH-CDR2 selected from SEQ ID Nos: 2, 10, 18, 104, 112, 128, 136, and 144; a VH-CDR3 selected from SEQ ID Nos: 3, 11, 19, 94, 96, 98, 105, and 113; and a CH1 domain. In some aspects, the antigen binding arm of the trivalent T cell engaging molecule further comprises a light chain comprising a VL domain comprising a VL-CDR1 selected from SEQ ID Nos: 4, 12, 20, 100, 108, 130, 138, and 146; a VL-CDR2 selected from SEQ ID Nos: 5, 13, 21, 101, 109, 131, 139, and 147; a VL-CDR3 selected from SEQ ID Nos: 6, 14, 22, 102, 110, 132, 140, and 148; and a light chain constant domain.

In some aspects, a trivalent T cell engaging molecule comprises an antigen binding arm that binds STEAP2 and comprises a heavy chain comprising a VH domain comprising an amino acid sequence acid having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149. In some aspects, a trivalent T cell engaging molecule comprises an antigen binding arm that binds STEAP2 and comprises a heavy chain comprising a VH domain comprising an amino acid sequence selected from SEQ ID Nos: 7, 15, 23, 95, 97, 99, 106, 114, 133, 141, and 149.

In some aspects, a trivalent T cell engaging molecule comprises an antigen binding arm that binds STEAP2 and comprises a heavy chain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 91. In some aspects, a trivalent T cell engaging molecule comprises an antigen binding arm that binds STEAP2 and comprises a heavy chain comprising an amino acid sequence set forth in SEQ ID NO: 91.

In some aspects, a trivalent T cell engaging molecule comprises an antigen binding arm that binds STEAP2 and comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 7, 15, 23, 95, 97, 99, 106, 114, 133, 141, and 149 and a heavy chain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 91. In some aspects, a trivalent T cell engaging molecule comprises an antigen binding arm that binds STEAP2 and comprises a heavy chain comprising a VH domain comprising an amino acid sequence selected from SEQ ID Nos: 7, 15, 23, 95, 97, 99, 106, 114, 133, 141, and 149 and a heavy chain comprising an amino acid sequence set forth in SEQ ID NO: 91.

In some aspects, the trivalent T cell engaging molecule further comprises at least one T cell binding arm that binds CD3 and comprises a heavy chain comprising a VH domain comprising a VH-CDR1 selected from SEQ ID Nos: 36, 40, and 44; a VH-CDR2 selected from SEQ ID Nos: 37, 41, and 45; a VH-CDR3 selected from SEQ ID Nos: 38, 42, and 46; and a CH1 domain. In some aspects, the T cell binding arm further comprises a light chain comprising a VL comprising a VL-CDR1 selected from SEQ ID Nos: 27 and 31; a VL-CDR2 selected from SEQ ID Nos: 28 and 32; a VL-CDR3 selected from SEQ ID Nos: 29 and 33; and a light chain constant domain. In some aspects, the trivalent T cell engaging molecule further comprises an Fc domain comprising a first Fc region and a second Fc region, each Fc region comprising a CH2 domain and a CH3 domain; further comprising at least one modification to promote heterodimerization.

In some aspects, the at least one T cell binding arm that binds CD3 comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 39, 43, and 47. In some aspects, the at least one T cell binding arm that binds CD3 comprises a VH domain comprising an amino acid sequence selected from SEQ ID Nos: 39, 43, and 47.

In some aspects, the at least one T cell binding arm that binds CD3 comprises a heavy chain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 79. In some aspects, the at least one T cell binding arm that binds CD3 comprises a heavy chain comprising an amino acid sequence set forth in SEQ ID NO: 79.

In some aspects, the trivalent T cell engaging molecule comprises a T cell binding arm that binds CD3 and comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID Nos: 39, 43, and 47 and a heavy chain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 79. In some aspects, the trivalent T cell engaging molecule comprises a T cell binding arm that binds CD3 and comprises a VH domain comprising an amino acid sequence selected from SEQ ID Nos: 39, 43, and 47 and a heavy chain comprising an amino acid sequence set forth in SEQ ID NO: 79.

In some aspects, the trivalent T cell engaging molecule further comprises at least one T cell binding arm that binds CD8 and comprises a heavy chain comprising a VH domain comprising a VH-CDR1 selected from SEQ ID Nos: 48 and 84; a VH-CDR2 selected from SEQ ID Nos: 49 and 85; a VH-CDR3 selected from SEQ ID Nos: 50 and 86; and a CH1 domain. In some aspects, the T cell binding arm further comprises a light chain comprising a VL comprising a VL-CDR1 set forth in SEQ ID NO: 51; a VL-CDR2 set forth in SEQ ID NO: 52; a VL-CDR3 set forth in SEQ ID NO: 53; and a light chain constant domain. In some aspects, the trivalent T cell engaging molecule further comprises an Fc domain comprising a first Fc region and a second Fc region, each Fc region comprising a CH2 domain and a CH3 domain; further comprising at least one modification to promote heterodimerization.

In some aspects, the at least one T cell binding arm that binds CD8 comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56. In some aspects, the at least one T cell binding arm that binds CD8 comprises a VH domain comprising an amino acid sequence set forth in SEQ ID NO: 56.

In some aspects, the at least one T cell binding arm that binds CD8 comprises a CH1 domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 57. In some aspects, the at least one T cell binding arm that binds CD8 comprises a CH1 domain comprising an amino acid sequence set forth in SEQ ID NO: 57.

In some aspects, the trivalent T cell engaging molecule comprises a T cell binding arm that binds CD8 and comprises a VH domain comprising an amino acid having at least about 80%, at least about 85%, at least about 90%, at least about 95%, at least about 96%, at least about 97%, at least about 98%, or at least about 99% sequence identity to an amino acid sequence set forth in SEQ ID NO: 56 and a CH1 domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 57. In some aspects, the trivalent T cell engaging molecule comprises a T cell binding arm that binds CD8 and comprises a VH domain comprising an amino acid set forth in SEQ ID NO: 56 and a CH1 domain comprising an amino acid sequence set forth in SEQ ID NO: 57.

In some aspects, the at least one T cell binding arm that binds CD8 comprises a VL domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54. In some aspects, the at least one T cell binding arm that binds CD8 comprises a VL domain comprising an amino acid set forth in SEQ ID NO: 54.

In some aspects, the at least one T cell binding arm that binds CD8 comprises a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 64. In some aspects, the at least one T cell binding arm that binds CD8 comprises a light chain constant domain comprising an amino acid sequence set forth in SEQ ID NO: 64.

In some aspects, the trivalent T cell engaging molecule comprises a T cell binding arm that binds CD8 and comprises a VL domain comprising an amino acid having at least about 80%, at least about 85%, at least about 90%, at least about 95%, at least about 96%, at least about 97%, at least about 98%, or at least about 99% sequence identity to an amino acid sequence set forth in SEQ ID NO: 54 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 64. In some aspects, the trivalent T cell engaging molecule comprises a T cell binding arm that binds CD8 and comprises a VL domain comprising an amino acid set forth in SEQ ID NO: 54 and a light chain constant domain comprising an amino acid sequence set forth in SEQ ID NO: 64.

In some aspects, a trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 25. In some aspects, a trivalent T cell engaging molecule comprises an amino acid sequence set forth in SEQ ID NO: 25.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 116. In some aspects, a T cell engaging molecule comprises a STEAP2 binding arm that comprises a light chain comprising the amino acid sequence set forth in SEQ ID NO: 116.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117. In some aspects, a T cell engaging molecule comprises a STEAP2 binding arm that comprises a light chain comprising the amino acid sequence set forth in SEQ ID NO: 117.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 118. In some aspects, a T cell engaging molecule comprises a STEAP2 binding arm that comprises a light chain comprising the amino acid sequence set forth in SEQ ID NO: 118.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 119. In some aspects, a T cell engaging molecule comprises a STEAP2 binding arm that comprises a light chain comprising the amino acid sequence set forth in SEQ ID NO: 119.

In some aspects, a trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 26. In some aspects, a trivalent T cell engaging molecule comprises an amino acid sequence set forth in SEQ ID NO: 26.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 120. In some aspects, a T cell engaging molecule comprises a STEAP2 binding arm that comprises a heavy chain comprising the amino acid sequence set forth in SEQ ID NO: 120.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 121. In some aspects, a T cell engaging molecule comprises a STEAP2 binding arm that comprises a heavy chain comprising the amino acid sequence set forth in SEQ ID NO: 121.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 122. In some aspects, a T cell engaging molecule comprises a STEAP2 binding arm that comprises a heavy chain comprising the amino acid sequence set forth in SEQ ID NO: 122.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 123. In some aspects, a T cell engaging molecule comprises a STEAP2 binding arm that comprises a heavy chain comprising the amino acid sequence set forth in SEQ ID NO: 123.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 124. In some aspects, a T cell engaging molecule comprises a STEAP2 binding arm that comprises a heavy chain comprising the amino acid sequence set forth in SEQ ID NO: 124.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 125. In some aspects, a T cell engaging molecule comprises a STEAP2 binding arm that comprises a heavy chain comprising the amino acid sequence set forth in SEQ ID NO: 125.

In some aspects, a T cell engaging molecule comprises a STEAP2 binding arm that comprises a heavy chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 126. In some aspects, a T cell engaging molecule comprises a STEAP2 binding arm that comprises a heavy chain comprising the amino acid sequence set forth in SEQ ID NO: 126.

In some aspects, a trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 35. In some aspects, a trivalent T cell engaging molecule comprises an amino acid sequence set forth in SEQ ID NO: 35.

In some aspects, a trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 55. In some aspects, a trivalent T cell engaging molecule comprises an amino acid sequence set forth in SEQ ID NO: 55.

In some aspects, a trivalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 58. In some aspects, a trivalent T cell engaging molecule comprises an amino acid sequence set forth in SEQ ID NO: 58.

In some aspects, a trivalent T cell engaging molecule comprises an amino acid sequence set forth in SEQ ID NO: 25, 26, 35, 55, and 58.

In some aspects, a trivalent T cell engaging molecule comprises a STEAP2 binding arm that is attached to a Fc region, a first T cell binding arm that binds CD3 and is attached to the Fc domain, and a heavy chain of a second T cell binding arm that binds CD8 is attached to the heavy chain of the first T cell binding arm that binds CD3.

In some aspects, the trivalent T cell engaging molecule that comprises two T cell binding arms has a higher efficacy in tumor cell cytotoxicity and lower cytokine release syndrome induction compared to a T cell engaging molecule that comprises one T cell binding arm, e.g., a CD3 binding arm.

### IX. Tetravalent T Cell Engaging Molecule

The present disclosure provides tetravalent T cell engaging molecules. Tetravalent T cell engaging molecules as described herein are capable of binding four different epitopes, either on the same or, in some aspects, different antigens. In some aspects, the tetravalent T cell binding molecules comprise at least two antigen binding arms and at least two T cell binding arms. In some aspects, the antigen binding arms are referred to herein as a “first antigen binding arm” and a “second antigen binding arm”, and the T cell binding arms are referred herein as a “first T cell binding arm” and a “second T cell binding arm”. In some aspects, an “antigen binding arm” comprises a heavy chain comprising a VH domain and CH1 domain and a light chain comprising a VL domain and a light chain constant domain, where the light chain constant domain is disulfide linked to the CH1 domain. In some aspects, a “T cell binding arm” comprises a heavy chain comprising a VH domain and CH1 domain and a light chain comprising a VL domain and a light chain constant domain, where the light chain constant domain is disulfide linked to the CH1 domain. In some aspects, a T cell binding arm comprises only a VH domain.

In some aspects, an antigen binding arm and/or at least one T cell binding arm in the tetravalent T cell engaging molecule further comprise an Fc region. In some aspects, an antigen binding arm and/or a T cell binding arm in the tetravalent T cell engaging molecule comprise complete heavy chains (i.e. a VH domain, CH1 domain, hinge region, CH2 domain and CH3 domain).

In some aspects, a tetravalent T cell engaging molecule comprises (i) a first antigen binding arm comprising a heavy chain comprising a VH and a heavy chain constant region comprising a CH1, CH2, and CH3 domain, and a light chain comprising a VL and a light chain constant domain; (ii) a first T cell binding arm comprising a heavy chain comprising a VH and a heavy chain constant region comprising a CH1, CH2, and CH3 domain, and a light chain comprising a VL and a light chain constant domain; (iii) a second antigen binding arm comprising a heavy chain comprising a VH and a CH1 domain and a light chain comprising a VL and a light chain constant domain; and (iv) a second T cell binding arm comprising a heavy chain comprising a VH domain; wherein the heavy chain of the second antigen binding arm is attached to the heavy chain of the first T cell binding arm through a peptide linker, and the VH domain of the heavy chain of the second T cell binding arm is attached to the heavy chain of the first antigen binding arm through a peptide linker. An exemplary tetravalent T cell engaging molecule is shown in FIG. 1D.

In some aspects, a light chain of a first and/or a second antigen binding arm is disulfide linked to a heavy chain of the first and/or second antigen binding arm in the tetravalent T cell engaging molecule via a native inter-chain disulfide bond (e.g., a disulfide bond present in an IgG antibody). In some aspects, a light chain of an antigen binding arm is disulfide linked to a heavy chain of the antigen binding arm in the tetravalent T cell engaging molecule via an engineered disulfide bond. In some aspects, a light chain of a first and/or second T cell binding arm is disulfide linked to a heavy chain of the first and/or second T cell binding arm in the tetravalent T cell engaging molecule via a native inter-chain disulfide. In some aspects, a light chain of a first and/or second T cell binding arm is disulfide linked to a heavy chain of the first and/or second T cell binding arm in the tetravalent T cell engaging molecule via an engineered disulfide bond.

In some aspects, a heavy chain of an antigen binding arm is attached to a heavy chain of another antigen binding arm via a peptide linker. In some aspects, a heavy chain of an antigen binding arm is attached to a T cell binding arm via a peptide linker. In some aspects, a peptide linker consists of 5 to 100 amino acids, 5 to 50 amino acids, 5 to 25 amino acids, or 5 to 15 amino acids. In some aspects, a peptide linker is formed mainly from glycine and serine amino acid residues and in some aspects comprises the amino acid sequences GGGGS or SGGGGS. In some aspects, the peptide linker comprises or consists of SEQ ID NO: 89. In some aspects, the linker comprises from 1 to about 10 copies of SEQ ID NO: 89. In some aspects, the linker comprises 2 copies of SEQ ID NO: 89.

In some aspects, a tetravalent T cell engaging molecule comprises (a) at least one first antigen binding arm that binds STEAP2 and comprises a heavy chain comprising a VH domain and a CH1 domain; and a light chain comprising a VL and a light chain constant domain; (b) at least one second antigen binding arm that binds STEAP2 and comprises a heavy chain comprising a VH domain and a CH1 domain; and a light chain comprising a VL and a light chain constant domain; (c) at least one T cell binding arm that binds a CD3 comprising a heavy chain comprising a VH domain and a CH1 domain; a light chain comprising a VL domain and a light chain constant domain; (d) at least one second T cell binding arm that binds a CD8 comprising a heavy chain comprising a VH domain and a CH1 domain; a light chain comprising a VL domain and a light chain constant domain; and (c) an Fc domain comprising a first Fc region and a second Fc region, each Fc region comprising a CH2 domain and a CH3 domain; further comprising at least one modification to promote heterodimerization.

In some aspects, one of the CH3 domains of the Fc region of the tetravalent T cell engaging molecule comprises a knob mutation, and the other CH3 domain comprises a hole mutation.

In some aspects, a CH1 domain and a light chain constant domain of one or more of each of an antigen binding arm and/or a T cell binding arm of the tetravalent T cell engaging molecule further comprise a charge pair substitution comprising a first charged amino acid substitution in the CH1 and a second charged amino acid substitution in the light chain constant domain, wherein the first charged amino acid substitution and the second charged amino acid substitution have an opposite charge.
(c) In some aspects, a light chain constant domain of one or more of each of an antigen binding arm and/or a T cell binding arm of the tetravalent T engaging molecule is a lambda light chain constant domain (CLλ) and the charge pair is a lambda charge pair comprising a positively charged amino acid residue selected from arginine, lysine or histidine, and a negatively charged amino acid residue selected from aspartic acid, glutamic acid, serine or threonine. In some aspects, the charged amino acids of a lambda charge pair are located at one or more of the following positions:
(i) position 117 in the CLλ and position 141 in the CH1 domain;
(ii) position 117 in the CLλ and position 185 in the CH1 domain;
(iii) position 119 in the CLλ and position 128 in the CH1 domain;
(iv) position 134 in the CLλ and position 128 in the CH1 domain;
(v) position 134 in the CLλ and position 145 in the CH1 domain;
(vi) position 134 in the CLλ and position 183 in the CH1 domain;
(vii) position 136 in the CLλ and position 185 in the CH1 domain;
(viii) position 178 in the CLλ and position 173 in the CH1 domain; and/or
(ix) position 117 in the CLλ and position 187 in the CH1 domain, wherein the numbering is according to the EU index.
(c) In some aspects, a light chain constant domain of one or more of each of an antigen binding arm and/or a T cell binding arm of the tetravalent T engaging molecule is a lambda light chain constant domain (CLλ) and the charge pair is a lambda charge pair, where ii) the charged amino acid at position 117 is arginine and the charged amino acid at position 141 is aspartic acid; the charged amino acid at position 117 is arginine and the charged amino acid at position 141 is glutamic acid; the charged amino acid at position 117 is arginine and the charged amino acid at position 141 is serine; the charged amino acid at position 117 is arginine and the charged amino acid at position 141 is threonine; the charged amino acid at position 117 is lysine and the charged amino acid at position 141 is aspartic acid; the charged amino acid at position 117 is lysine and the charged amino acid at position 141 is glutamic acid; the charged amino acid at position 117 is lysine and the charged amino acid at position 141 is serine; or the charged amino acid at position 117 is lysine and the charged amino acid at position 141 is threonine;
(ii) the charged amino acid at position 117 is arginine and the charged amino acid at position 185 is aspartic acid; the charged amino acid at position 117 is arginine and the charged amino acid at position 185 is glutamic acid; the charged amino acid at position 117 is arginine and the charged amino acid at position 185 is serine; the charged amino acid at position 117 is arginine and the charged amino acid at position 185 is threonine; the charged amino acid at position 117 is lysine and the charged amino acid at position 185 is aspartic acid; the charged amino acid at position 117 is lysine and the charged amino acid at position 185 is glutamic acid; the charged amino acid at position 117 is lysine and the charged amino acid at position 185 is serine; or the charged amino acid at position 117 is lysine and the charged amino acid at position 185 is threonine;
(iii) the charged amino acid at position 119 is arginine and the charged amino acid at position 128 is aspartic acid; the charged amino acid at position 119 is arginine and the charged amino acid at position 128 is glutamic acid; the charged amino acid at position 119 is arginine and the charged amino acid at position 128 is serine; the charged amino acid at position 119 is arginine and the charged amino acid at position 128 is threonine; the charged amino acid at position 119 is lysine and the charged amino acid at position 128 is aspartic acid; the charged amino acid at position 119 is lysine and the charged amino acid at position 128 is glutamic acid; the charged amino acid at position 119 is lysine and the charged amino acid at position 128 is serine; or the charged amino acid at position 119 is lysine and the charged amino acid at position 128 is threonine;
(iv) the charged amino acid at position 134 is arginine and the charged amino acid at position 128 is aspartic acid; the charged amino acid at position 134 is arginine and the charged amino acid at position 128 is glutamic acid; the charged amino acid at position 134 is arginine and the charged amino acid at position 128 is serine; the charged amino acid at position 134 is arginine and the charged amino acid at position 128 is threonine; the charged amino acid at position 134 is lysine and the charged amino acid at position 128 is aspartic acid; the charged amino acid at position 134 is lysine and the charged amino acid at position 128 is glutamic acid; the charged amino acid at position 134 is lysine and the charged amino acid at position 128 is serine; or the charged amino acid at position 134 is lysine and the charged amino acid at position 128 is threonine;
(v) the charged amino acid at position 134 is arginine and the charged amino acid at position 145 is aspartic acid; the charged amino acid at position 134 is arginine and the charged amino acid at position 145 is glutamic acid; the charged amino acid at position 134 is arginine and the charged amino acid at position 145 is serine; the charged amino acid at position 134 is arginine and the charged amino acid at position 145 is threonine; the charged amino acid at position 134 is lysine and the charged amino acid at position 145 is aspartic acid; the charged amino acid at position 134 is lysine and the charged amino acid at position 145 is glutamic acid; the charged amino acid at position 134 is lysine and the charged amino acid at position 145 is serine; or the charged amino acid at position 134 is lysine and the charged amino acid at position 145 is threonine;
(vi) the charged amino acid at position 134 is arginine and the charged amino acid at position 183 is aspartic acid; the charged amino acid at position 134 is arginine and the charged amino acid at position 183 is glutamic acid; the charged amino acid at position 134 is arginine and the charged amino acid at position 183 is serine; the charged amino acid at position 134 is arginine and the charged amino acid at position 183 is threonine; the charged amino acid at position 134 is lysine and the charged amino acid at position 183 is aspartic acid; the charged amino acid at position 134 is lysine and the charged amino acid at position 183 is glutamic acid; the charged amino acid at position 134 is lysine and the charged amino acid at position 183 is serine; or the charged amino acid at position 134 is lysine and the charged amino acid at position 183 is threonine;
(vii) the charged amino acid at position 136 is arginine and the charged amino acid at position 185 is aspartic acid; the charged amino acid at position 136 is arginine and the charged amino acid at position 185 is glutamic acid; the charged amino acid at position 136 is arginine and the charged amino acid at position 185 is serine; the charged amino acid at position 136 is arginine and the charged amino acid at position 185 is threonine; the charged amino acid at position 136 is lysine and the charged amino acid at position 185 is aspartic acid; the charged amino acid at position 136 is lysine and the charged amino acid at position 185 is glutamic acid; the charged amino acid at position 136 is lysine and the charged amino acid at position 185 is serine; or the charged amino acid at position 136 is lysine and the charged amino acid at position 185 is threonine;
(viii) the charged amino acid at position 178 is arginine and the charged amino acid at position 173 is aspartic acid; the charged amino acid at position 178 is arginine and the charged amino acid at position 173 is glutamic acid; the charged amino acid at position 178 is arginine and the charged amino acid at position 173 is serine; the charged amino acid at position 178 is arginine and the charged amino acid at position 173 is threonine; the charged amino acid at position 178 is lysine and the charged amino acid at position 173 is aspartic acid; the charged amino acid at position 178 is lysine and the charged amino acid at position 173 is glutamic acid; the charged amino acid at position 178 is lysine and the charged amino acid at position 173 is serine; or the charged amino acid at position 178 is lysine and the charged amino acid at position 173 is threonine; and/or
(ix) the charged amino acid at position 117 is arginine and the charged amino acid at position 187 is aspartic acid; the charged amino acid at position 117 is arginine and the charged amino acid at position 187 is glutamic acid; the charged amino acid at position 117 is arginine and the charged amino acid at position 187 is serine; the charged amino acid at position 117 is arginine and the charged amino acid at position 187 is threonine; the charged amino acid at position 117 is lysine and the charged amino acid at position 187 is aspartic acid; the charged amino acid at position 117 is lysine and the charged amino acid at position 187 is glutamic acid; the charged amino acid at position 117 is lysine and the charged amino acid at position 187 is serine; or the charged amino acid at position 117 is lysine and the charged amino acid at position 187 is threonine.

In some aspects, a light chain constant domain of one or more of each an antigen binding arm and/or a T cell binding arm of the tetravalent T engaging molecule is a kappa light chain constant domain (CLκ) and the charge pair is a kappa charge pair comprising a positively charged amino acid residue selected from arginine, lysine or histidine, and a negatively charged amino acid residue selected from aspartic acid, glutamic acid, serine or threonine, wherein the kappa charge pair is located at position 133 in the CLκ and position 183 in the CH1 domain.

In some aspects, a light chain constant domain of one or more of each an antigen binding arm and/or a T cell binding arm of the tetravalent T engaging molecule is a kappa light chain constant domain (CLκ) and the charge pair is a kappa charge pair wherein the charged amino acid at position 133 is glutamic acid and the charged amino acid at position 183 is lysine; or the charged amino acid at position 133 is lysine and the charged amino acid at position 183 is glutamic acid.

In some aspects, a light chain constant domain of one T cell binding arm of the tetravalent T engaging molecule is a CLλ and the charge pair is a lambda charge pair as described herein and the light chain constant domain of an antigen binding arm and/or a second T cell binding arm of the tetravalent T engaging molecule is a lambda or a kappa charge pair and the antigen binding arm comprises a charged amino acid in the CH1 domain that is of a same charge as a charged amino acid in the CH1 domain of the second T cell binding arm.

In some aspects, a light chain constant domain of one T cell binding arm of the tetravalent T engaging molecule is a CLλ and the charge pair is a lambda charge pair as described herein and the light chain constant domain of an antigen binding arm and a second T cell binding arm is a kappa charge pair and a charged amino acid in the CH1 domain of the antigen binding arm is of a same charge as a charged amino acid in the CH1 domain of the second T cell binding arm.

In some aspects, a CH1 domain of an antigen binding arm or a T cell binding arm of a tetravalent T cell engaging molecule is capable of being linked to a light chain constant domain through an engineered disulfide link.

In some aspects, a CH1 domain that is capable of being linked to the light chain constant domain through an engineered disulfide link comprises (i) a substitution of a native cysteine to a non-cysteine amino acid and (ii) a substitution of a native non-cysteine amino acid to a cysteine; and the light chain constant domain comprises (i) a substitution of a native cysteine to a non-cysteine amino acid and (ii) a substitution of a native non-cysteine amino acid to a cysteine; wherein the substituted cysteine of the light chain constant domain and the substituted cysteine of the CH1 domain can form a disulfide bond.

In some aspects, a CH1 domain that is capable of being linked to the light chain constant domain through an engineered disulfide link comprises (i) a substitution of a native cysteine to a non-cysteine amino acid at position 220, and (ii) a substitution of a native non-cysteine amino acid to a cysteine at position 126; and the light chain constant domain comprises (i) a substitution of a native cysteine to a non-cysteine amino acid at position 212, and (ii) a substitution of a native non-cysteine amino acid to a cysteine at position 122, wherein the cysteine at position 126 of the CH1 domain and the cysteine at position 122 of the light chain constant domain can form a disulfide bond; wherein the numbering is according to the EU index.

In some aspects, a CH1 domain that is capable of being linked to the light chain constant domain through an engineered disulfide link comprises (i) a substitution of a native cysteine to a non-cysteine amino acid at position 220, and (ii) a substitution of a native non-cysteine amino acid to a cysteine at position 126; and the light chain constant domain comprises (i) a substitution of a native cysteine to a non-cysteine amino acid at position 214, and (ii) a substitution of a native non-cysteine amino acid to a cysteine at position 121, wherein the cysteine at position 126 of the CH1 domain and the cysteine at position 121 of the light chain constant domain can form a disulfide bond; wherein the numbering is according to the EU index.

In some aspects, a tetravalent T cell engaging molecule comprises a first antigen binding arm that binds STEAP2 and comprises a heavy chain comprising a VH domain comprising a VH-CDR1 selected from SEQ Id NOs: 1, 9, 17, 103, 111, 127, 135, and 143; a VH-CDR2 selected from SEQ Id NOs: 2, 10, 18, 104, 112, 128, 136, and 144; a VH-CDR3 selected from SEQ Id NOs: 3, 11, 19, 94, 96, 98, 105, 113, 129, 137, and 145; and a CH1 domain. In some aspects, the first antigen binding arm of the tetravalent T cell engaging molecule further comprises a light chain comprising a VL domain comprising a VL-CDR1 selected from SEQ Id NOs: 4, 12, 20, 100, 108, 130, 138, and 146; a VL-CDR2 selected from SEQ Id NOs: 5, 13, 21, 101, 109, 131, 139, and 147; a VL-CDR3 selected from SEQ Id NOs: 6, 14, 22, 102, 110, 132, 140, and 148; and a light chain constant domain.

In some aspects, a tetravalent T cell engaging molecule comprises a first antigen binding arm that binds STEAP2 and comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 7, 15, and 23. In some aspects, a tetravalent T cell engaging molecule comprises a first antigen binding arm that binds STEAP2 and comprises a heavy chain comprising a VH domain comprising an amino acid sequence selected from SEQ Id NOs: 7, 15, 23, 95, 97, 99, 106, 114, 133, 141, and 149.

In some aspects, a tetravalent T cell engaging molecule comprises a first antigen binding arm that binds STEAP2 and comprises a CH1 domain comprising an amino acid sequence having at least about 80%, at least about 85%, at least about 90%, at least about 95%, at least about 96%, at least about 97%, at least about 98%, or at least about 99% sequence identity to set forth in SEQ ID NO: 67. In some aspects, a tetravalent T cell engaging molecule comprises a first antigen binding arm that binds STEAP2 and comprises a CH1 domain comprising an amino acid sequence set forth in SEQ ID NO: 67.

In some aspects, a tetravalent T cell engaging molecule comprises a first antigen binding arm that binds STEAP2 and comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 7, 15, 23, 95, 97, 99, 106, 114, 133, 141, and 149 and a CH1 domain comprising an amino acid sequence having at least about 80%, at least about 85%, at least about 90%, at least about 95%, at least about 96%, at least about 97%, at least about 98%, or at least about 99% sequence identity to set forth in SEQ ID NO: 67. In some aspects, a tetravalent T cell engaging molecule comprises a first antigen binding arm that binds STEAP2 and comprises a heavy chain comprising a VH domain comprising an amino acid sequence selected from SEQ Id NOs: 7, 15, 23, 95, 97, 99, 106, 114, 133, 141, and 149 and a CH1 domain comprising an amino acid sequence set forth in SEQ ID NO: 67.

In some aspects, a tetravalent T cell engaging molecule comprises a first antigen binding arm that binds STEAP2 and comprises a light chain comprising a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 8, 16, 24, 107, 115, 134, 142, and 150. In some aspects, a tetravalent T cell engaging molecule comprises a first antigen binding arm that binds STEAP2 and comprises a light chain comprising a VL domain comprising an amino acid sequence selected from SEQ Id NOs: 8, 16, 24, 107, 115, 134, 142, and 150.

In some aspects, a tetravalent T cell engaging molecule comprises a first antigen binding arm that binds STEAP2 and comprises a light chain constant domain comprising an amino acid sequence having at least about 80%, at least about 85%, at least about 90%, at least about 95%, at least about 96%, at least about 97%, at least about 98%, or at least about 99% sequence identity to set forth in SEQ ID NO: 63. In some aspects, a tetravalent T cell engaging molecule comprises a first antigen binding arm that binds STEAP2 and comprises a light chain constant domain comprising an amino acid sequence set forth in SEQ ID NO: 63.

In some aspects, a tetravalent T cell engaging molecule comprises a first antigen binding arm that binds STEAP2 and comprises a light chain comprising a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 8, 16, 24, 107, 115, 134, 142, and 150 and a light chain constant domain comprising an amino acid sequence having at least about 80%, at least about 85%, at least about 90%, at least about 95%, at least about 96%, at least about 97%, at least about 98%, or at least about 99% sequence identity to set forth in SEQ ID NO: 63. In some aspects, a tetravalent T cell engaging molecule comprises a first antigen binding arm that binds STEAP2 and comprises a heavy chain comprising a VH domain comprising an amino acid sequence selected from SEQ Id NOs: 8, 16, 24, 107, 115, 134, 142, and 150 and a light chain constant domain comprising an amino acid sequence set forth in SEQ ID NO: 63.

In some aspects, the tetravalent T cell engaging molecule further comprises a first T cell binding arm that binds CD3 and comprises a heavy chain comprising a VH domain comprising a VH-CDR1 selected from SEQ Id NOs: 36, 40, and 44; a VH-CDR2 selected from SEQ Id NOs: 37, 41, and 45; a VH-CDR3 selected from SEQ Id NOs: 38, 42, and 46; and a CH1 domain. In some aspects, the first T cell binding arm further comprises a light chain comprising a VL comprising a VL-CDR1 selected from SEQ Id NOs: 27 and 31; a VL-CDR2 selected from SEQ Id NOs: 28 and 32; a VL-CDR3 selected from SEQ Id NOs: 29 and 33; and a light chain constant domain. In some aspects, the tetravalent T cell engaging molecule further comprises an Fc domain comprising a first Fc region and a second Fc region, each Fc region comprising a CH2 domain and a CH3 domain; further comprising at least one modification to promote heterodimerization.

In some aspects, a first T cell binding arm of a T cell engaging molecule comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 39, 43, and 47. In some aspects, a T cell binding arm of a T cell engaging molecule comprises a VH domain comprising an amino acid sequence selected from SEQ Id NOs: 39, 43, and 47.

In some aspects, a first T cell binding arm of a T cell engaging molecule comprises a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 79. In some aspects, a first T cell binding arm of a T cell engaging molecule comprises a heavy chain constant domain comprising an amino acid sequence set forth in SEQ ID NO: 79.

In some aspects, a first T cell binding arm of a T cell engaging molecule comprises a heavy chain comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 39, 43, and 47 and a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 79.

In some aspects, a first T cell binding arm of a T cell engaging molecule comprises a heavy chain comprising a VH domain comprising an amino acid sequence selected from SEQ Id NOs: 39, 43, and 47 and a heavy chain constant domain comprising an amino acid sequence set forth in SEQ ID NO: 79.

In some aspects, a first T cell binding arm of the T cell engaging molecule comprises a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 30 and 34. In some aspects, a first cell binding arm of a T cell engaging molecule comprises a light chain comprising a VL domain comprising an amino acid sequence selected from SEQ Id NOs: 30 and 34.

In some aspects, a first T cell binding arm of a T cell engaging molecule comprises a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 61. In some aspects, a first T cell binding arm of a T cell engaging molecule comprises a light chain constant domain comprising an amino acid sequence set forth in SEQ ID NO: 61.

In some aspects, a first T cell binding arm of a T cell engaging molecule comprises a light chain comprising a VL domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ Id NOs: 30 and 34 and a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 61.

In some aspects, a first T cell binding arm of a T cell engaging molecule comprises a light chain comprising a VL domain comprising an amino acid sequence selected from SEQ Id NOs: 30 and 34 and a light chain constant domain comprising an amino acid sequence set forth in SEQ ID NO: 61.

In some aspects, the tetravalent T cell engaging molecule further comprises a second T cell binding arm that binds CD8 and comprises a heavy chain comprising a VH domain comprising a VH-CDR1 selected from SEQ Id NOs: 48 and 84; a VH-CDR2 selected from SEQ Id NOs: 49 and 85; a VH-CDR3 selected from SEQ Id NOs: 50 and 86; and a CH1 domain. In some aspects, the T cell binding arm further comprises a light chain comprising a VL comprising a VL-CDR1 set forth in SEQ ID NO: 51; a VL-CDR2 set forth in SEQ ID NO: 52; a VL-CDR3 set forth in SEQ ID NO: 53; and a light chain constant domain. In some aspects, the tetravalent T cell engaging molecule further comprises an Fc domain comprising a first Fc region and a second Fc region, each Fc region comprising a CH2 domain and a CH3 domain; further comprising at least one modification to promote heterodimerization.

In some aspects, a second T cell binding arm of a tetravalent T cell engaging molecule comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 56. In some aspects, a T cell binding arm of a T cell engaging molecule comprises a VH domain comprising an amino acid sequence set forth in SEQ ID NO: 56.

In some aspects, a second T cell binding arm of a tetravalent T cell engaging molecule comprises a CH1 domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 57. In some aspects, a T cell binding arm of a T cell engaging molecule comprises a CH1 domain comprising an amino acid sequence set forth in SEQ ID NO: 57.

In some aspects, a second T cell binding arm of a tetravalent T cell engaging molecule comprises a VL domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 54. In some aspects, a T cell binding arm of a T cell engaging molecule comprises a VL domain comprising an amino acid sequence set forth in SEQ ID NO: 54.

In some aspects, a second T cell binding arm of a tetravalent T cell engaging molecule comprises a light chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 64. In some aspects, a T cell binding arm of a T cell engaging molecule comprises a light chain constant domain comprising an amino acid sequence set forth in SEQ ID NO: 64.

In some aspects, the tetravalent T cell engaging molecule further comprises a second T cell binding arm that binds CD8 and comprises a heavy chain comprising a VH domain comprising a VH-CDR1 set forth in SEQ ID NO: 84; a VH-CDR2 set forth in SEQ ID NO: 85; a VH-CDR3 set forth in SEQ ID NO: 86.

In some aspects, a second T cell binding arm of a tetravalent T cell engaging molecule comprises a VH domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 83. In some aspects, a T cell binding arm of a T cell engaging molecule comprises a VH domain comprising an amino acid sequence set forth in SEQ ID NO: 83.

In some aspects, a tetravalent T cell engaging molecule comprises a CD8 binding arm comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 83; and an antigen binding arm comprising a VH domain comprising an amino acid sequence having at least about 80%, at least about 85%, at least about 90%, at least about 95%, at least about 96%, at least about 97%, at least about 98%, or at least about 99% sequence identity to an amino acid sequence selected from SEQ ID NO: 7, 15, and 23 and a heavy chain constant domain comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 88.

In some aspects, a tetravalent T cell engaging molecule comprises a CD8 binding arm comprising a VH domain comprising an amino acid sequence set forth in SEQ ID NO: 83; and an antigen binding arm comprising a VH domain comprising an amino acid sequence selected from SEQ ID NO: 7, 15, 23, 95, 97, 99, 106, 114, 133, 141, and 149 and a heavy chain constant domain comprising an amino acid sequence set forth in SEQ ID NO: 88.

In some aspects, a tetravalent T cell engaging molecule comprises a CD8 binding arm and an antigen binding arm comprising an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 87.

In some aspects, a tetravalent T cell engaging molecule comprises a CD8 binding arm and an antigen binding arm comprising an amino acid sequence set forth in SEQ ID NO: 87.

In some aspects, a tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 25. In some aspects, a tetravalent T cell engaging molecule comprises an amino acid sequence set forth in SEQ ID NO: 25.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 116. In some aspects, a T cell engaging molecule comprises a STEAP2 binding arm that comprises a light chain comprising the amino acid sequence set forth in SEQ ID NO: 116.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 117. In some aspects, a T cell engaging molecule comprises a STEAP2 binding arm that comprises a light chain comprising the amino acid sequence set forth in SEQ ID NO: 117.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 118. In some aspects, a T cell engaging molecule comprises a STEAP2 binding arm that comprises a light chain comprising the amino acid sequence set forth in SEQ ID NO: 118.

In some aspects, a T cell engaging molecule comprises a STEAP2 binding arm that comprises a light chain comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 119. In some aspects, a T cell engaging molecule comprises a STEAP2 binding arm that comprises a light chain comprising the amino acid sequence set forth in SEQ ID NO: 119.

In some aspects, a tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 35. In some aspects, a tetravalent T cell engaging molecule comprises an amino acid sequence set forth in SEQ ID NO: 35.

In some aspects, a tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 81. In some aspects, a tetravalent T cell engaging molecule comprises an amino acid sequence set forth in SEQ ID NO: 81.

In some aspects, a tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 87. In some aspects, a tetravalent T cell engaging molecule comprises an amino acid sequence set forth in SEQ ID NO: 87.

In some aspects, a tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 25, an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 35; an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 81; and an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 87.

In some aspects, a tetravalent T cell engaging molecule comprises an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 151, an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 152; an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 153; and an amino acid sequence having at least about 80%, at least about 85%, at least about 90%, at least about 95%, at least about 96%, at least about 97%, at least about 98%, or at least about 99% sequence identity to an amino acid sequence set forth in SEQ ID NO: 154.

In some aspects, a tetravalent T cell engaging molecule comprises an amino acid sequence set forth in SEQ ID NO: 25, 35, 81, and 87. In some aspects, a tetravalent T cell engaging molecule comprises an amino acid sequence set forth in SEQ ID NO: 151, 152, 153, 154, and 152.

In some aspects, the tetravalent T cell engaging molecule that comprises two STEAP2 binding arms, a CD3 binding arm and a CD8 binding arm has a higher cytotoxicity against STEAP2 expressing tumor cells and a lower cytokine release syndrome induction compared to a T cell engaging molecule that comprises two STEAP2 binding arms and a CD3 binding arm and/or a trivalent T cell engaging molecule that comprises one STEAP2 binding arm, a CD3 binding arm and a CD8 binding arm.

### X. Nucleic Acids, Vectors, Host Cells, and Kits

Also provided herein is one or more nucleic acid(s) encoding a T cell engaging molecule described herein. In some aspects, a nucleic acid(s) is/are purified or isolated, e.g. from other nucleic acid, or naturally-occurring biological material. The skilled person would have no difficulty in preparing such nucleic acid molecules using methods well-known in the art.

In some aspects, the one or more nucleic acids encode a first, second and/or third light chain as described herein and/or a first, second and/or third CH1 domain as described herein. The one or more nucleic acid(s) encoding the first or second CH1 may further encode other heavy chain domains, e.g. the hinge, CH2 and CH3, and may encode a complete heavy chain.

In some aspects, provided is one or more vector(s) comprising nucleic acid(s) encoding a T cell engaging molecule described herein. Suitable vectors can be chosen or constructed, containing appropriate regulatory sequences, including promoter sequences, terminator fragments, polyadenylation sequences, enhancer sequences, marker genes and other sequences as appropriate. In some aspects, a vector contains appropriate regulatory sequences to drive the expression of the nucleic acid in a host cell. Vectors may be plasmids, viral e.g. phage, or phagemid, as appropriate.

In some aspects, a T cell engaging molecule is produced from one or more light chain vectors and one or more heavy chain vectors. A light chain vector may contain the nucleic acid encoding a first light chain, a nucleic acid encoding a second light chain acid, and a nucleic acid encoding a third light chain, which may be present on the vector as separate cassettes (e.g. each operably connected to a different promoter). Alternatively, separate vectors may be used, i.e. one containing the nucleic acid encoding a first light chain, one containing the nucleic acid encoding a second light chain and one containing the nucleic acid encoding a third light chain.

A heavy chain vector may contain may be used to encode both a first CH1 and first VH (and first Fc region, if present) and second CH1 and second VH (and second Fc region, if present), which may be present on the vector as separate cassettes. Alternatively, separate vectors may be used, i.e. one containing the nucleic acid encoding a first CH1 and first VH (and first Fc region, if present) and one containing the nucleic acid encoding a second CH1 and second VH (and second Fc region, if present). In some aspects, a T cell engaging molecule comprises a CH1 of a second T cell binding arm fused to a heavy chain of either the first T cell binding arm or an antigen binding arm. In some aspects, a T cell engaging molecule comprises a VH of a second T cell binding arm fused to a heavy chain of either the first T cell binding arm or an antigen binding arm. Accordingly, if a second T cell binding arm is fused to a first antigen binding arm then the third CH1 and third VH will be encoded by the nucleic acid encoding the first CH1 and first VH, and if a second T cell binding arm is fused to a second antigen binding arm then the third CH1 and third VH will be encoded by the nucleic acid encoding the second CH1 and second VH.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. In some aspects, the host cell is a mammalian cell, such as a CHO, NS0, or HEK cell, for example a HEK293 cell. In some aspects, the host cell is a CHO cell.

Also provided are kits according to the present disclosure that comprise a T cell engaging molecule as described herein and optionally instruction, e.g., administration instructions. In some aspects, a kit comprises multiple packages of single-dose pharmaceutical compositions each containing an effective amount of a T cell engaging molecule for a single administration in accordance with the administration instructions. In some aspects, the kit also includes one or more additional cancer therapeutics. In some aspects, the kit includes instruments or devices necessary for administering the pharmaceutical composition(s).

### XI. Methods of Producing T Cell Engaging Molecules

Also provided herein is a method of producing the T cell engaging molecules described herein.

In some aspects, the method comprises
a) expressing a first, second and third light chain and a first, second and third CH1 and/or heavy chain in a host cell;
b) allowing the first light chain to pair with the first CH1 and/or heavy chain so as to form the first binding arm, allowing the second light chain to pair with the second CH1 and/or heavy chain so as to form the second binding arm, allowing the third light chain to pair with the third CH1 and/or heavy chain, and allowing the first, second and third binding arms to form the T cell engaging molecule; and
c) purifying T cell engaging molecules from the host cell.

Expressing a first, second and third light chain and first, second and third CH1 and/or heavy chain in a host cell may comprise introducing nucleic acids or vectors into host cells (e.g. CHO cells) using suitable techniques as described above. The host cell may then be cultured using suitable techniques, such that the light chain and heavy chain polypeptides pair and form the first, second, and third binding arms. The various light chains and heavy chain polypeptides associate with each other (e.g. through inter-chain disulfide bonds formed between native cysteines, and/or through cysteines engineered into the T cell engaging molecules as described herein) and the heavy chains associate with each other (e.g. through inter-chain disulfide bonds formed between cysteines in the two Fc regions). As described herein, the presence of the lambda and kappa charge pairs, and (if present) engineered disulfides, encourages the correct heavy chain/light chain pairs to form in the T cell engaging molecule.

Techniques for the purification of recombinant T cell engaging molecules are well-known in the art and include, for example high performance liquid chromatography, fast protein liquid chromatography, ion exchange chromatography, and affinity chromatography, e.g. using Protein A or Protein L or by binding to an affinity tag. In some aspects, purification is carried out using affinity chromatography (e.g. Protein A affinity chromatography). In some aspects, purification further comprises (e.g. in addition to Protein A chromatography) light chain affinity chromatography. As described herein, light chain affinity chromatography can be used to selectively purify T cell engaging molecules containing both CLκ and CLλ and can therefore be used to improve production of T cell engaging molecules.

In some embodiments, less than 25%, less than 20%, less than 15%, or less than 10%, less than 5%, less than 4%, less than 3%, less than 2%, or less than 1% of the light chains in the T cell engaging molecules are mispaired (i.e. paired with a CH1 from a different antigen binding arm) following purification (e.g. by protein A affinity chromatography, or following protein A affinity chromatography and light chain affinity chromatography). Methods for determining the correct light chain pairing are known in the art and include mass spectrometry analysis and microfluidics-based electrophoresis, as described herein. In some cases the method comprises measuring the correct light chain pairing.

The method may also comprise formulating T cell engaging molecule into a pharmaceutical composition, optionally with a pharmaceutically acceptable excipient or other substance as described below.

### XII. Methods of Treatment

The T cell engaging molecules described herein may be used for therapeutic applications, such as in the treatment of cancer, e.g., prostate cancer.
(c) In some aspects, a T cell engaging molecule as described herein may be used in a method of treatment of the human or animal body. Related aspects of the disclosure provided a T cell engaging molecule described herein for use as a medicament,
(ii) a T cell engaging molecule described herein for use in a method of treatment of a disease or disorder,
(iii) a T cell engaging molecule described herein in the manufacture of a medicament for use in the treatment of a disease or disorder; and,
(iv) a method of treating a disease or disorder in an individual, wherein the method comprises administering to the individual a therapeutically effective amount of a T cell engaging molecules as described herein.

The individual may be a patient, and in some aspects, a human patient, e.g. a patient suffering from prostate cancer.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment. In some aspects, the method is a method of treating cancer.

Treatment as a prophylactic measure (i.e. prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

In some aspects, a method of treatment as described comprises administering T cell engaging molecule to a subject in need thereof. In some aspects, the method further comprises administering at least one further treatment to the subject in addition to the T cell engaging molecule. The T cell engaging molecule described herein may thus be administered to a subject alone or in combination with one or more other treatments. Where the T cell engaging molecules is administered to the subject in combination with another treatment, the additional treatment may be administered to the individual concurrently with, sequentially to, or separately from the administration of the T cell engaging molecule. Where the additional treatment is administered concurrently with the T cell engaging molecule, T cell engaging molecule and additional treatment may be administered to the subject as a combined preparation. For example, the additional therapy may be a known therapy or therapeutic agent for the disease to be treated, e.g., cancer.

In some aspects, the T cell engaging molecules are administered in the form of a pharmaceutical composition, which may comprise at least one component in addition to the T cell engaging molecule. Another aspect of the disclosure therefore provides a pharmaceutical composition comprising a T cell engaging molecule as described herein. A method comprising formulating a T cell engaging molecule into a pharmaceutical composition is also provided.

Pharmaceutical compositions may comprise, in addition to the T cell engaging molecule, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

In some aspects, T cell engaging molecules may be provided in a lyophilized form for reconstitution prior to administration. For example, lyophilized T cell engaging molecules may be re-constituted in sterile water and mixed with saline prior to administration to a subject.

Administration may be in a “therapeutically effective amount”, this being sufficient to show benefit in a subject. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners.

### EXAMPLES

### Example 1—Materials and Methods Cloning of T Cell Engaging Molecules

For construction of T cell engaging molecules with charge pair mutations in heavy chain-light chain interfaces, the pDuet-Heavy and pDuet-Light plasmids described in (Mazor et. al mAbs 2015) were used as backbone vectors. The pDuet-Heavy vector contained two human gamma1 heavy chain (HC) cassettes to support HC heterodimerization, where one heavy chain cassette carried the “Hole” set of mutations (T366S/L368A/Y407V) and a stabilizing mutation (Y349C) in CH3 domain, while the other heavy chain cassette carried the complement “Knob” mutation (T366W) and a stabilizing mutation (S354C) in CH3. The cassettes can also be readily used in the reverse order.

The pDuet-Light vector contained two human light chain (LC) cassettes, where one light chain carried a kappa constant domain (Cκ), while the other carried a lambda constant domain (Cλ). The pDuet-Heavy and pDuet-Light vectors also contained the mutations to remove the native interchain disulfide bond in CH1/Ck and provide the alternative disulfide bond which is denoted as “V12 DS” or “V12” herein. Further, the mutations F126C/C220V were introduced in the CH1 domain of the “Knob” heavy chain, and mutations S122C/C212V were introduced in Cλ.

The mutations of the “Knob-and-Hole” set and the stabilizing/alternative disulfide bonds utilized herein were provided as examples. One skilled in the art can use any other combinations of mutations for “Knob-and-Hole” technique and/or stabilizing/alternative disulfide bonds known in the field to support heavy chain heterodimerization.

For construction of the pDuet-Heavy vector with charge mutations, the “Hole” heavy chain was cloned into the pDuet-Heavy vector by a synthesized DNA fragment of VH-CH1-CH2-CH3 domains containing the above-mentioned mutations for “Hole” heavy chain using restriction cloning technique by BssHII/HindIII. Optionally, the “Hole” heavy chain contained the charge mutation S183K in CH1 domain. The “Knob” heavy chain was cloned into the vector by a synthesized DNA fragment of VH-CH1-CH2-CH3 domains containing the above-mentioned mutations for “Knob” heavy chain using restriction cloning technique by BsrGI/EcoRI. Optionally, the “Knob” heavy chain contained one of the charge mutations in CH1 domain: L128D, L128E, L128S, L128T, A141D, A141E, A141S, A141T, L145D, L145E, L145S, L145T, S183D, V185D, V185E, V185S, V185T, V173D, V173E, V173S, and V173T.

### Cell Binding Assays

Cell based binding assays were performed on BD FACSymphony™ A5 Cell Analyzer. To determine binding affinity of AZD6621, C4-2 cells and Jurkat (TCR neg) CD8ab+ cells expressing Steap2 and CD8ab respectively were used for the assay. The cells were harvested and resuspended in FACS buffer (DPBS-1X, 2% FBS, & 5 mM EDTA) at 2×10e6 cells/ml. Cells were seeded in U-bottom Tissue Culture treated 96 Well-plate at 50 ul/well making the cell number at 100,000 per well. The test articles (AZD6621) titration were prepared (2×) using FACS buffer with the starting working concentration of 600 nM and with 1:4 dilution up to 8 titration points. Cells (50 ul/well) were then combined with primary antibody (50 ul/well) titration and incubated at 4° C. for 1 hour. After incubation, the cells were washed 2× using 200 ul of FACS buffer and resuspended with freshly prepared FACS buffer (100 ul/well) containing secondary antibody (Anti-H+L: Goat anti-Human IgG (H+L) Cross-Adsorbed Secondary Antibody, Alexa Fluor 647 (Invitrogen #A-21445) at 1:400 dilution) & DAPI (Live/Dead stain). The secondary antibody was incubated at 4° C. for 1 hour. After incubation, the cells were washed 2× using 200 ul of FACS buffer and resuspended with 100 ul of FACS buffer. The data was analyzed with FlowJo v 10.6.1. The target receptor binding was determined using geometric mean calculation on FlowJo software detecting the positive APC signal in each well. The cells were gated to only include singlet and live cells for detecting positive signal and plotted using GraphPad Prism v 9.0.0.

### Cell Viability and T Cell Activation FACS Assays

Redirected T-cell cytotoxicity and T cell activation assays were performed on BD FACSymphony™ A5 Cell Analyzer. To determine cytotoxicity, target cells were first stained with CellTracker™ Violet (Thermo Fisher Scientific), according to the manufacturer's instructions. Cells were then combined with human PBMCs (peripheral blood mononuclear cells, effector cell:target cell ratio at 10:1) in RPMI1640 supplemented with 10% heat-inactivated FBS and 50 μM of 2-Mercaptoethanol and seeded in 96-well plates. T cell engaging molecules at various concentrations were added to triplicate samples, and the cells were incubated for at 37° C. and 5% CO₂ in a humidified incubator. For detection of CD4 and CD8 T-cell activation, the cells were stained with anti-CD4, anti-CD8, anti-CD25 and anti-CD69 antibodies (all from BioLegend, San Diego, CA, USA). The data were analyzed with FlowJo v 10.6.1. Number of depleted target cells has been determined with the following formula: % Lysis=100−(viable cells of treatment group/viable cells of untreated control group×100) and plotted using GraphPad Prism v 9.0.0.

To determine the impact of target antigen density on cytotoxicity, target cells were seeded on xCELLigence plates for 24 hours. Human PBMCs (peripheral blood mononuclear cells) or CD3+ T cells were then added at an effector cell:target cell ratio of 10:1 or 5:1 respectively in RPMI1640 supplemented with 10% heat-inactivated FBS, 50 M of 2-Mercaptoethanol and 1% penicillin/streptomycin. T cell engaging molecules at various concentrations were added to duplicate samples, and the cells were incubated for at 37° C. and 5% CO₂ in a humidified incubator for 3 days. Cell index was measured using the xCELLigence reader as per manufacturer's instructions and cytotoxicity was calculated against untreated samples and plotted in GraphPad Prism to calculate EC50 concentrations. Supernatants were collected at 72 hours and cytokines analyzed using MSD kits (Meso Scale Discovery). For detection of STEAP2, the target cells and Quantum Simply Cellular anti-human IgG beads (Bang laboratories) were stained with anti-STEAP2 antibody and the receptor density measured according to the manufacturer's instructions. For detection of CD4 and CD8 T-cell activation, the cells were stained with anti-CD3, anti-CD4, anti-CD8 and anti-CD25 antibodies and analyzed by flow cytometry.

### Treg Activation Assay

Regulatory T cells (Tregs) were isolated using the EasySep Human CD4+CD127lowCD25+ Regulatory T cell isolation kit (Stemcell) and expanded with anti-CD3/CD28 dynabeads (Invitrogen) and 100 IU IL-2 for 6 days. Tregs were rested overnight, labeled with CellTrace CFSE and incubated with CellTrace Violet-labeled CD3+ T cells from a different donor at various Treg:T effector ratios in the presence of 10,000 C4-2 tumor cells. T cell engagers were added at various concentrations and the cells were incubated for at 37° C. and 5% CO₂ in a humidified incubator for 3 days. Viability was measured using CellTiter-Glo (Promega) and cytotoxicity calculated against untreated samples.

For assessment of activation, Tregs were isolated using the EasySep Human CD4+CD127lowCD25+ Regulatory T cell isolation kit, labeled with CellTrace Violet and incubated with CellTrace CFSE-labeled CD8+ T cells from a different donor at various Treg:T effector ratios in the presence of 10,000 C4-2 tumor cells. T cell engagers were added at various concentrations and the cells were incubated for at 37° C. and 5% CO₂ in a humidified incubator for 3 days. T cells were harvested and stained with anti-CD3, anti-CD4, anti-CD8, anti-CD25 and anti-granzyme B antibodies and analyzed by flow cytometry.

### C4-2 Serial Killing Assay

C4-2 cells were seeded on xCELLigence plates for 24 hours. Human PBMCs were then added at an effector cell:target cell ratio of 10:1 in RPMI1640 supplemented with 10% heat-inactivated FBS, 50 M of 2-Mercaptoethanol and 1% penicillin/streptomycin. 40A3LO12-TED4-K17/E75 at various concentrations was added to duplicate samples, and the cells were incubated for at 37° C. and 5% CO₂ in a humidified incubator for 3 days (Kill 1). PBMCs were then collected, washed, transferred to a new plate of C4-2 cells and treated with T cell engager again for 3-4 days for each subsequent kill. Cell index was measured using the xCELLigence reader as per manufacturer's instructions and cytotoxicity was calculated against untreated samples.

### C4-2 Spheroid Cytotoxicity Assay

mKate2-expressing C4-2 cells were seeded into 96-well ultra-low attachment plates. 48 hours later CD3+ T cells were added at an effector to target ratio of 7.5:1 in RPMI1640 supplemented with 10% heat-inactivated FBS, 50 M of 2-Mercaptoethanol and 1% penicillin/streptomycin. T cell engaging molecules at various concentrations were added to duplicate samples, and the cells were incubated in an Incucyte S3 (Sartorius) situated in an humidified incubator at 37° C. and 5% CO₂. Cytotoxicity was calculated from a loss of fluorescence using the following calculation: % Cytotoxicity=100−(fluorescence of treated group/fluorescence of untreated control group×100) and plotted using GraphPad Prism.

### C4-2 Bystander Killing Assay

Different ratios of STEAP2-positive mKate2-expressing C4-2 tumor cells (C4-2 WT) and STEAP2-knockout GFP-expressing C4-2 tumor cells were seeded plates for 24 hours. Human PBMCs from 3 donors were then added at an effector cell:target cell ratio of 10:1 in RPMI1640 supplemented with 10% heat-inactivated FBS, 50 M of 2-Mercaptoethanol and 1% penicillin/streptomycin. 40A3LO12-TED4-K17/E75 at three concentrations was added to duplicate samples and the cells were incubated in an Incucyte S3 (Sartorius) situated in an humidified incubator at 37° C. and 5% CO₂ for 3 days. Cytotoxicity was calculated from a loss of either red or green fluorescence using the following calculation: % Cytotoxicity=100-(fluorescence of treated group/fluorescence of untreated control group×100) and plotted using GraphPad Prism.

### Prostate Cancer Patient-Derived Xenograft Organoid Assay

Single cells from dissociated patient-derived xenograft organoids were plated into Gri3D plates (Sun Bioscience) and allowed to form organoids over 12 days. T cells isolated from 3 human PBMC donors were added at a ratio of 5:1 (organoid seeding density). 40A3LO12-TED4-K17/E75 at 6 concentrations (10 nM-0.0032 nM) or NIP228-TED4-K17/E75 at 2 concentrations were added to duplicate samples and the cells were incubated in an Incucyte (Sartorius) situated in an humidified incubator at 37° C. and 5% CO₂ for 2 days. Cytotoxicity and T cell activation were calculated by Flow cytometry, where % Cytotoxicity=100−(% Live tumour treated group/% Live tumour untreated control group×100) and plotted using GraphPad Prism.

### Prostate Tumour Slice Assay

6 mm punch biopsies were generated from fresh prostate tumours from resections and embedded into 4% agarose and cut into 300 μm thick slices. These slices were incubated for 3 days with 46.5 nM 40A3LO12-TED4-K17/E75. Supernatants were then collected and cytokines analyzed using MSD kits (Meso Scale Discovery).

### Human PBMC Engrafted Mouse Models

Male NOG mice were s.c. implanted with 1e⁷ LNCaP tumor cells, i.v. engrafted on day 13 with 1e⁷ human PBMCs and i.p. injected on days 14, 17, 21, 24, 28 and 31 with 5 mg/kg 30D12-TED3-K29/SN75 V12 or NIP228-TED3-K29/SN75 V12 T cell engagers 24h after an i.p. injection of 20 mg/kg of anti-mouse CD16/CD32 (BioXcell). Tumor volume was measured two times per week using calipers.

Male NSG MHCI/II double knock out mice were s.c. implanted with 5e⁶ C4-2 tumor cells, i.v. engrafted on day 3 with 1e⁷ human PBMCs, and i.p. injected on days 18, 21, 25, 28, 32 and 35 with 6.65 mg/kg of 30D12-TED3-K29/SN75 or 5 mg/kg of 40A3LO12-TED2-K29/SN75 24h after an i.p. injection of 20 mg/kg of anti-mouse CD16/CD32 (BioXcell). Tumor volume was measured three times per week using calipers.

Male NSG MHCI/II double knock out mice were irradiated with 1 Gy, i.v. implanted with 3e⁶ luciferase-expressing C4-2 tumor cells, i.v. engrafted on day 24 with 1.5e⁷ human PBMCs, and treated i.p. on days 34, 38, 42, and 46 with 1 or 1.6 mg/kg of T cell engagers and bioluminescence was measured using an IVIS in vivo imaging system (Xenogen). Retroorbital bleeds were collected 6 hours after the first dose for serum analysis of TNFα using an MSD kit (Meso Scale Discovery) as per manufacturer's instructions.

Male NSG MHCI/II double knock out mice were s.c. implanted with 22Rv1 tumor cells, i.v. engrafted 7 days prior with 1e⁷ human PBMCs, and i.p. injected on days 3, 6, 10, 14, 17 and 21 with either NIP228-TED4-K17/E75 or 40A3LO12-TED4-K17/E75. Tumor volume was measured three times per week using calipers. On day 11, blood and tumors were harvested and stained with anti-CD3, anti-CD4, anti-CD8, anti-CD25 and anti-CD69 antibodies and analyzed by flow cytometry.

Female NSG MHCI/II double knock out mice were intratibially implanted with luciferase-expressing C4-2 tumor cells, i.v. engrafted on day 4 with 1e⁷ human PBMCs, and treated i.p. on days 14, 18, 21, 25, 28 and 32 with 1 mg/kg of either NIP228-TED4-K17/E75 or 40A3LO12-TED4-K17/E75 and bioluminescence was measured using an IVIS in vivo imaging system (Xenogen).

Male NSG MHCI/II double knock out mice were s.c. implanted with fragments of a prostate cancer patient-derived xenograft model, i.v. engrafted on day 35 with 1e⁷ human PBMCs, and i.p. injected on days 42, 45, 49, 52, 56 and 59 with 40A3LO12-TED4-K17/E75. Tumor volume was measured two times per week using calipers.

### Human PBMC/C4-2 Admix Mouse Model

NSG MHCI/II double knock out mice were s.c. implanted with a mix of 5e⁶ C4-2 tumor cells and 0.4e⁶ human PBMCs and i.p. injected on days 1, 4, 8, 11, 15, 18, 50, 53, 57, 60, 64 and 67 with 1.33 mg/kg of 30D12-TED3-K29/SN75 V12 or 1 mg/kg of 40A3LO12-TED2-K29/SN75 24h after an i.p. injection of 20 mg/kg of anti-mouse CD16/CD32 (BioXcell). Tumor volume was measured three times per week using calipers.

### Human CD34+ Stem Cell Engrafted Mouse Model

Newborn male NSG mice were irradiated and engrafted with human CD34+ stem cells. At 14 weeks of age, mice were s.c. implanted with 5e⁶ C4-2 tumor cells and i.p. injected on days 18, 22, 26 and 29 with 6.67 mg/kg of 30D12-TED3-K29/SN75 V12 and 20 mg/kg of anti-mouse CD16/CD32 (BioXcell). Tumor volume was measured using calipers.

### Example 2—Cell Binding

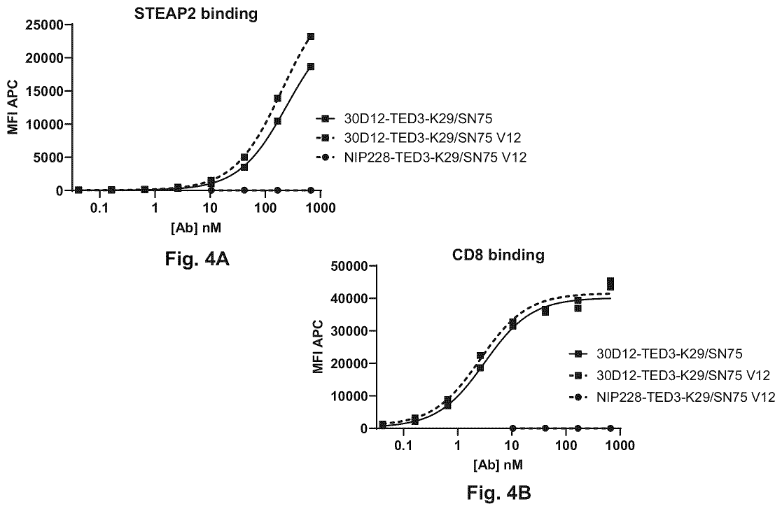

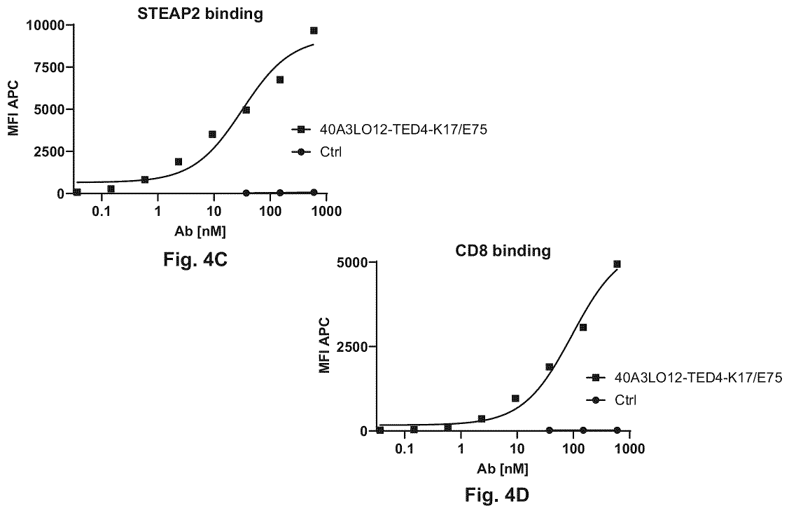

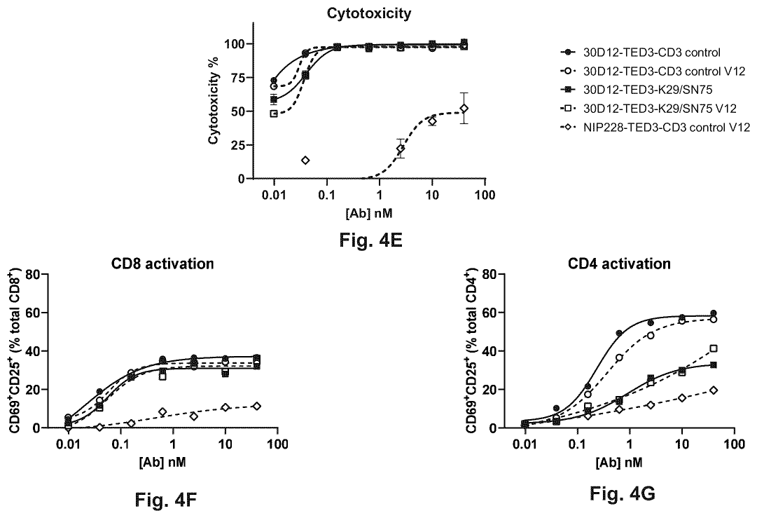

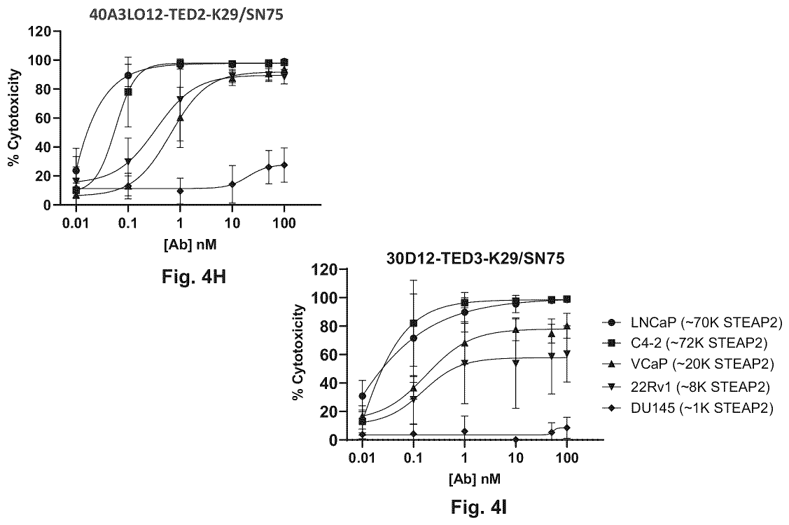

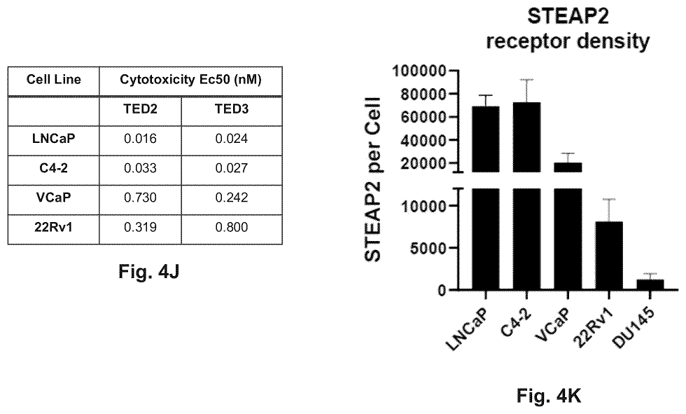

Comparable binding to C4-2 cells was observed for 30D12-G96P-TED3-K29/SN75 and 30D12-G96P-TED3-K29/SN75 V12 molecules compared to control NIP228-TED3-K29/SN75 V12 molecule (FIG. 4A). Further, comparable binding to CD8 T cells was observed for 30D12-G96P-TED3-K29/SN75 and 30D12-G96P-TED3-K29/SN75 V12 molecules compared to control NIP228-TED3-K29/SN75 V12 molecule (FIG. 4B). Binding to C4-2 cells was observed for 40A3LO12-TED4-K17/E75 compared to control (FIG. 4C). Further, binding to CD8 T cells was observed for 40A3LO12-TED4-K17/E75 compared to control (FIG. 4D).

### Example 3—Cytotoxicity, Cytokine Release and T Cell Activation

Comparable cytotoxicity of C4-2 cells was observed for 30D12-TED3-CD3 control and 30D12-TED3-CD3 control V12 as well as for 30D12-TED3-K29/SN75 and 30D12-TED3-K29/SN75 V12 molecules (FIG. 4E).

Comparable T cell activation was observed for 30D12-TED3-CD3 control and 30D12-TED3-CD3 control V12 as well as for 30D12-TED3-K29/SN75 and 30D12-TED3-K29/SN75 V12 molecules. All STEAP2-targeted T cell engagers similarly led to higher activation of CD8 T cells compared to the NIP228-TED3-CD3 control V12 molecule (FIG. 4F); but 30D12-TED3-K29/SN75 and 30D12-TED3-K29/SN75 V12 CD8-guided molecules showed lower CD4 T cell activation levels compared to 30D12-TED3-CD3 control and 30D12-TED3-CD3 control V12 molecules (FIG. 4G).

Cytotoxicity was also tested using cells with different levels of STEAP2 expression (FIG. 4K). 40A3LO12-TED2-K29/SN75 demonstrated high cytotoxicity against high STEAP2 expressing cells LNCap, C4-2, VCaP, 22Rv1 cells and low cytotoxicity against low STEAP2 expressing DU145 cells (FIG. 4H).

30D12-TED3-K29/SN75 demonstrated high cytotoxicity against high STEAP2 expressing cells LNCaP, C4-2, VCaP, 22Rv1 cells and no cytotoxicity against low STEAP2 expressing DU145 cells (FIG. 4I). Accordingly, the EC50 values were generally similar for TED3 and TED2 T cell engagers against high STEAP2 expressing cells (FIG. 4J).

Cytotoxicity and induction of cytokine release (IL-6 and TNFα) were also tested in LNCaP, C4-2, and VCaP cells. 40A3LO12-TED2-K29/SN75 and 30D12-TED3-K29/SN75 T cell engagers demonstrated comparable cytotoxicity (FIG. 4L). However, CD8-guided 30D12-TED3-K29/SN75 consistently induced lower IL-6 and TNFα levels compared to 40A3LO12-TED2-K29/SN75 (FIG. 4L).

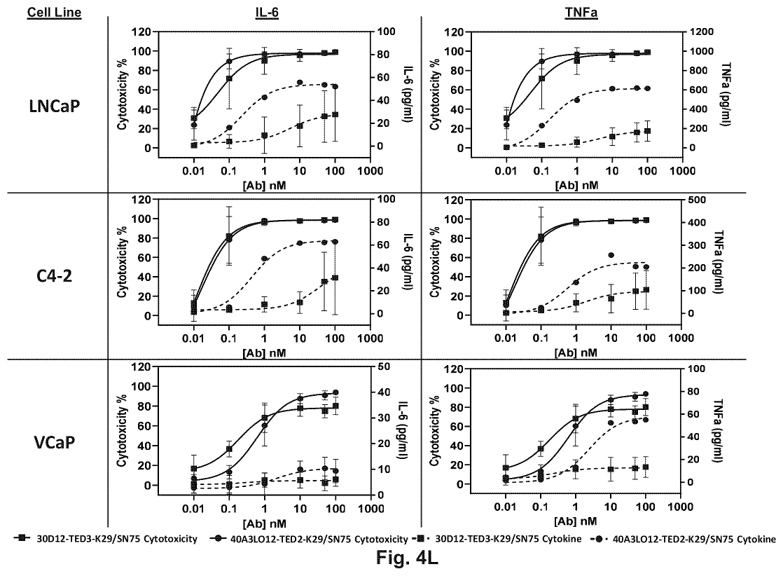

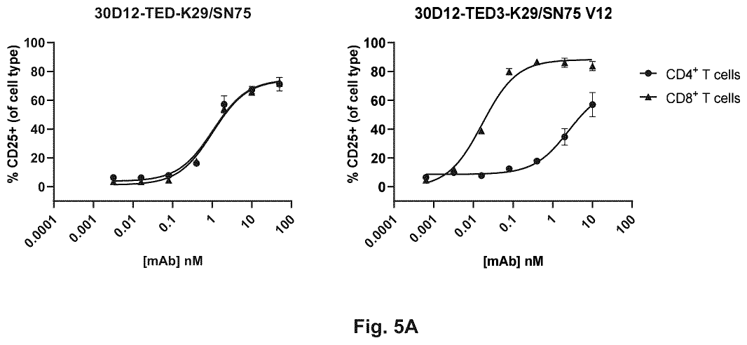

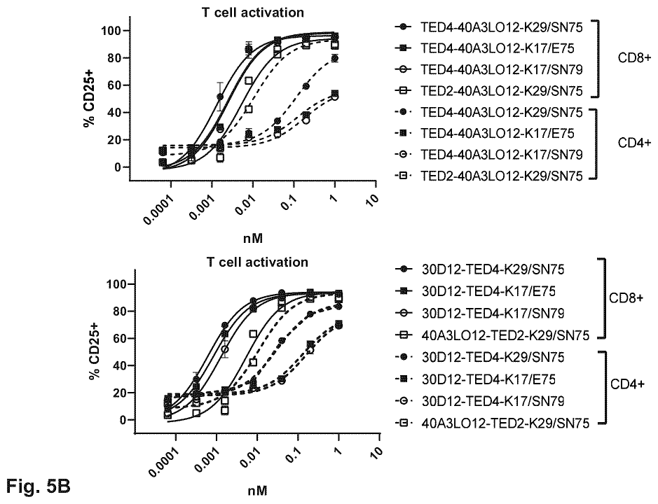

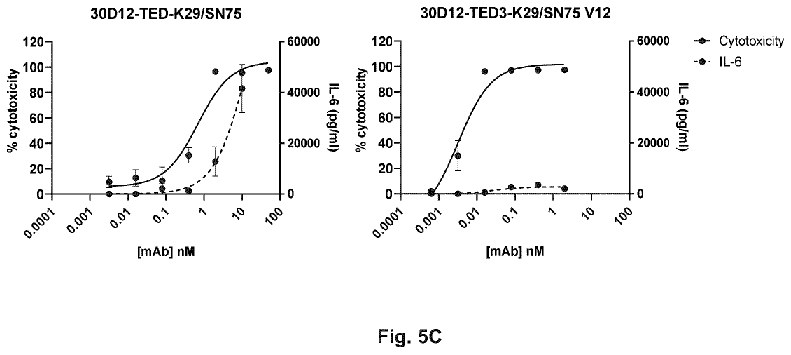

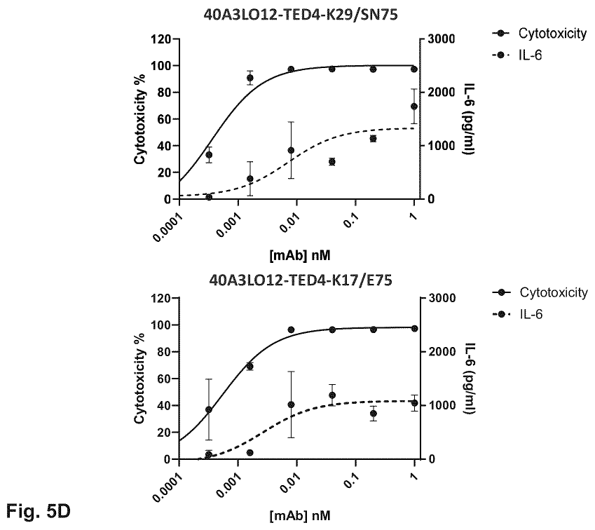

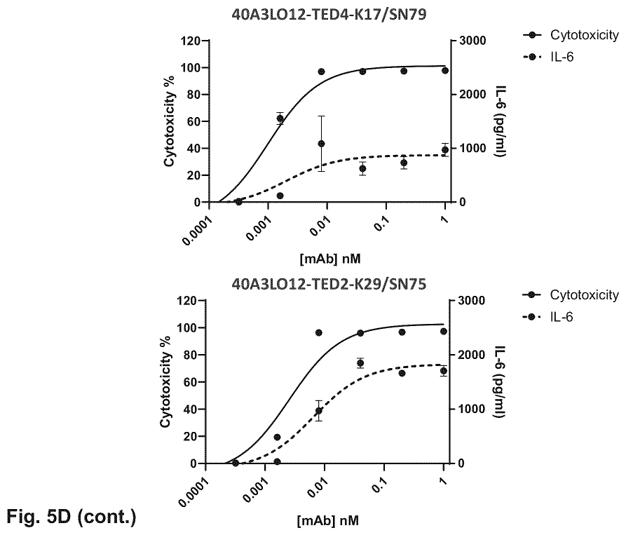

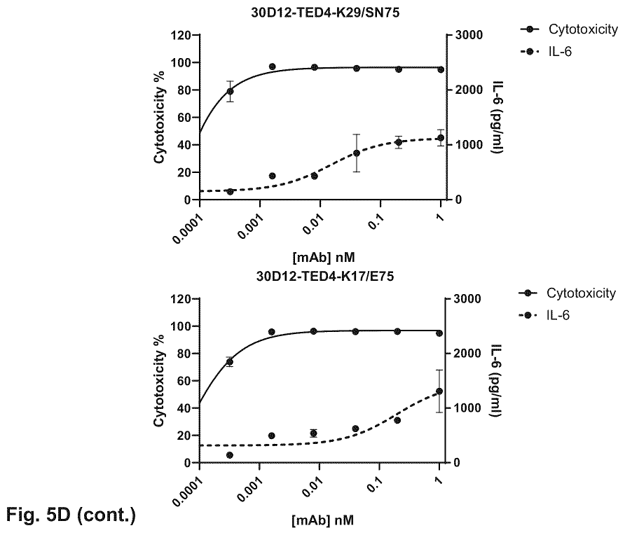

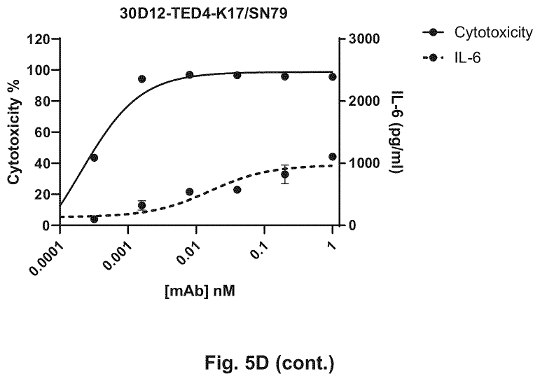

CD8-guided 30D12-TED3-K29/SN75 V12 also demonstrated preferential activation of CD8 T cells over CD4 T cells which was not observed with 30D12-TED-K29/SN75 molecule (FIG. 5A). All variants of tetravalent STEAP2 T cell engaging molecules also demonstrated preferential activation of CD8 T cells over CD4 T cells which was not observed with 40A3LO12-TED2-K29/SN75 molecule (FIG. 5B).

Furthermore, in a C4-2 cytotoxicity assay, 30D12-TED3-K29/SN75 showed lower levels of IL-6 release compared to 30D12-TED-K29/SN75 molecule at similar levels of cytotoxicity (FIG. 5C). All variants of tetravalent STEAP2 T cell engaging molecules also demonstrated lower levels of IL-6 release compared to 40A3LO12-TED2-K29/SN75 molecule at similar levels of cytotoxicity (FIG. 5D).

### Example 4—Regulatory T Cell Activation and Suppression of Cytotoxicity

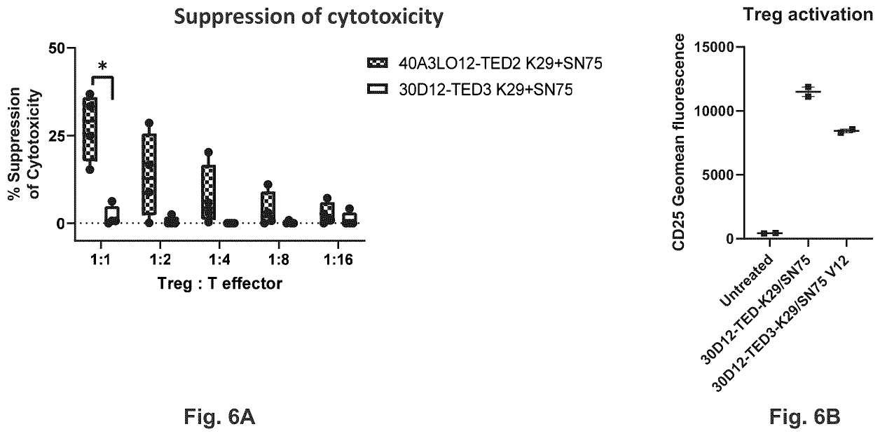

Regulatory T cells (Tregs), T effector cells and C4-2 tumor cells were incubated at different Treg:T effector ratios with 40A3LO12-TED2-K29/SN75 and 30D12-TED3-K29/SN75 V12 T cell engagers. Reduced suppression of cytotoxicity was observed in the presence of 30D12-TED3-K29/SN75 V12 compared to 40A3LO12-TED2-K29/SN75 (FIG. 6A). 30D12-TED3-K29/SN75 V12 also showed reduced Treg activation compared to an 30D12-TED-K29/SN75 (FIG. 6B).

### Example 5—Fluorescent C4-2 Spheroid Model

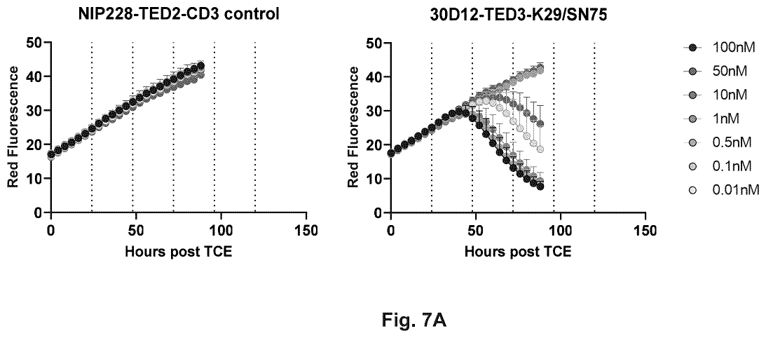

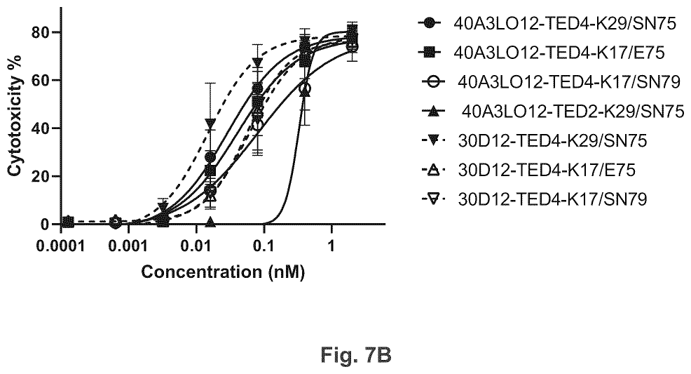

mKate2-expressing C4-2 spheroids were incubated with effector cells and T cell engaging molecules. Tumor cell viability was measured as red fluorescence. 30D12-TED3-K29/SN75 triggered increased tumor cell killing compared to a NIP228-TED2-CD3 control molecule. The assay was performed at increasing concentrations of 30D12-TED3-K29/SN75 and a dose-dependent increase in cytotoxicity was observed (FIG. 7A). Furthermore, all variants of tetravalent (TED4) T cell engaging molecules triggered increased tumor cell killing compared to 40A3LO12-TED2-K29/SN75 (FIG. 7B).

### Example 6—Human PBMC Engrafted Mouse Models

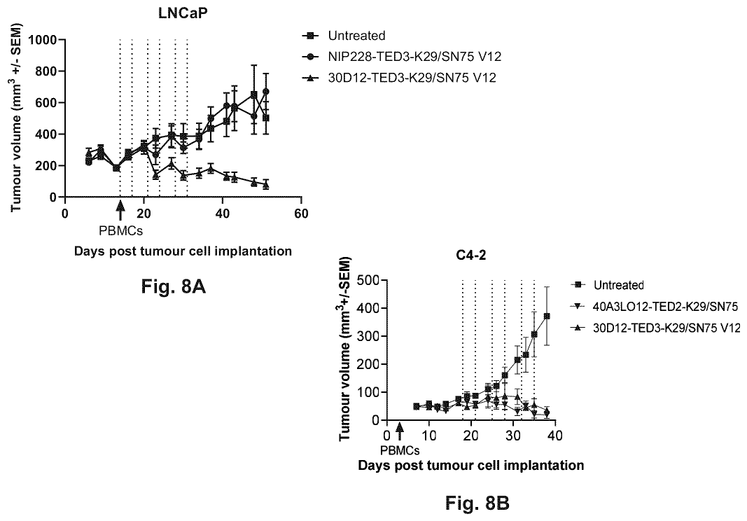

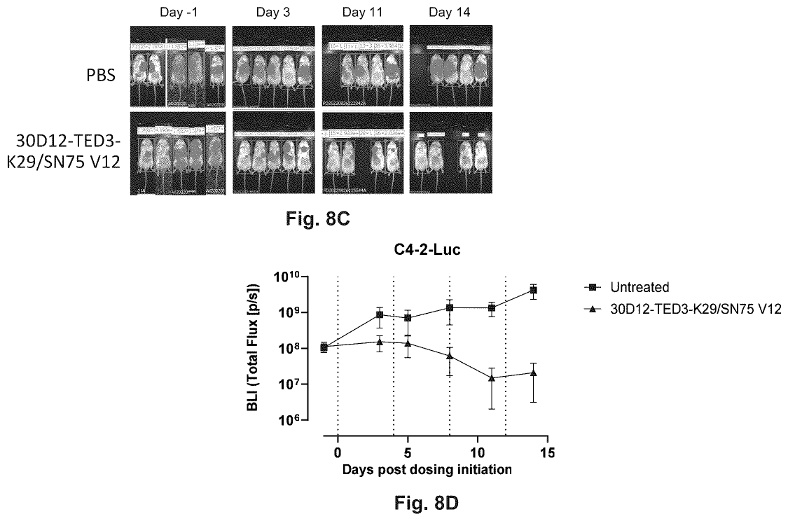

NOG mice were engrafted with human PBMCs, s.c. injected with LNCaP tumor cells, and treated with 30D12-TED3-K29/SN75 V12 T cell engager or untargeted NIP228-TED3-K29/SN75 V12 molecule. Mice receiving 30D12-TED3-K29/SN75 V12 showed reduced LNCaP tumor volumes compared to untreated control mice and NIP228-TED3-K29/SN75 V12 treated mice. Four of 10 mice receiving 30D12-TED3-K29/SN75 V12 showed complete responses (FIG. 8A).

NSG MHCI/II double knock out mice were engrafted with human PBMCs, s.c. injected with C4-2 tumor cells, and treated with 30D12-TED3-K29/SN75 V12 or 40A3LO12-TED2-K29/SN75 T cell engagers. Mice receiving either 30D12-TED3-K29/SN75 V12 or 40A3LO12-TED2-K29/SN75 showed reduced C4-2 tumor volumes compared to untreated control mice. Two of 7 mice receiving 30D12-TED3-K29/SN75 V12 and five out of 7 mice receiving 40A3LO12-TED2-K29/SN75 showed complete responses (FIG. 8B).

NSG MHCI/II double knock out mice i.v. implanted with luciferase expressing C4-2 cells, engrafted with human PBMCs and treated with 30D12-TED3-K29/SN75 V12 showed reduced bioluminescence over 14 days compared to untreated mice (FIGS. 8C and 8D).

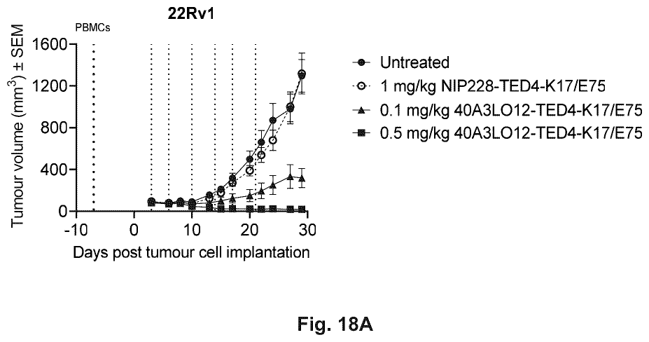

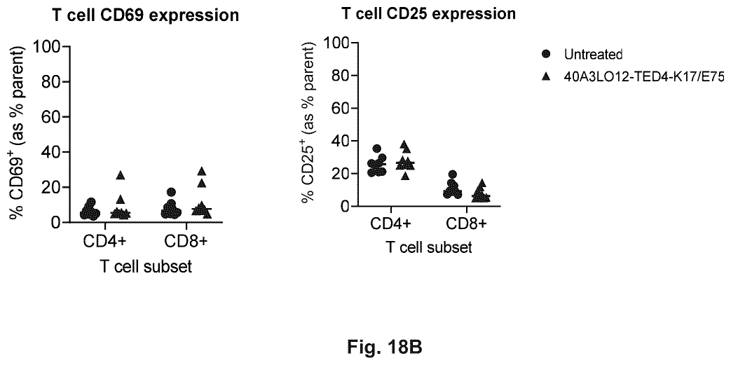

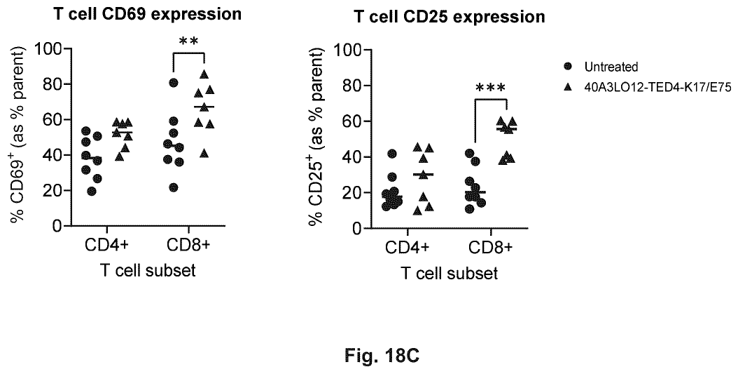

NSG MHCI/II double knock out mice were engrafted with human PBMCs, s.c. injected with 22Rv1 tumor cells, and treated with 40A3LO12-TED4-K17/E75 or untargeted NIP228-TED4-K17/E75 molecule. Mice receiving 40A3LO12-TED4-K17/E75 showed reduced 22Rv1 tumor volumes compared to control mice (FIG. 18A).

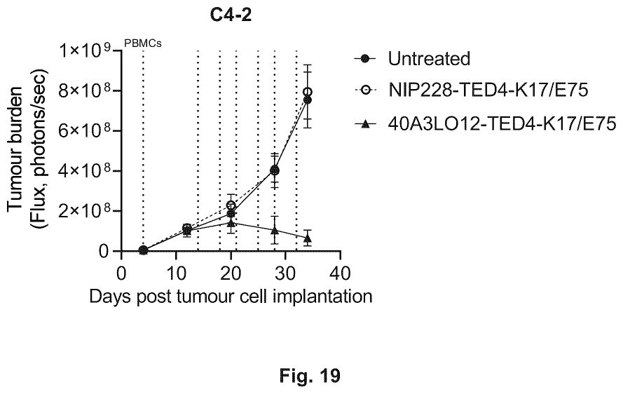

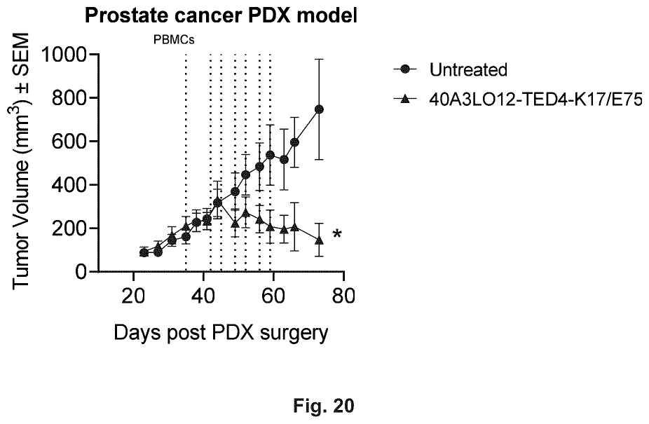

NSG MHCI/II double knock out mice were engrafted with human PBMCs, intratibially injected with luciferase-expressing C4-2 tumor cells and treated with 1 mg/kg of 40A3LO12-TED4-K17/E75 or untargeted NIP228-TED4-K17/E75 molecule. Tumor burden was measured using bioluminescence. Mice receiving 40A3LO12-TED4-K17/E75 showed reduced C4-2 tumor burden compared to control mice (FIG. 19).

NSG MHCI/II double knock out mice were engrafted with human PBMCs, s.c. implanted with a fragment of a prostate cancer patient-derived xenograft model and treated with 5 mg/kg of 40A3LO12-TED4-K17/E75. Mice receiving 40A3LO12-TED4-K17/E75 showed reduced tumor volumes compared to control mice (FIG. 18A).

### Example 7—Reduced Cytokine Release With Optimized CD3 Binding Arm

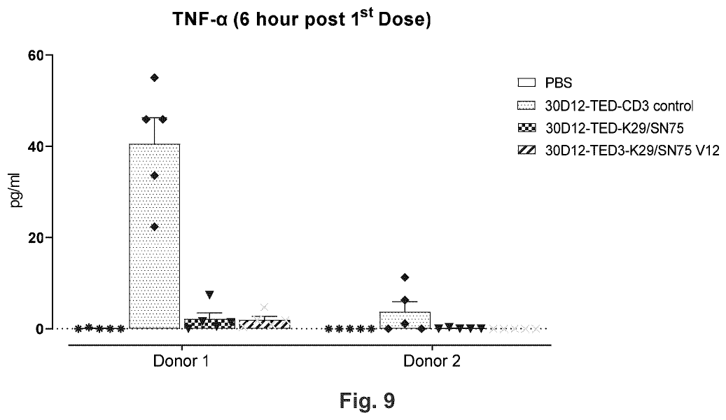

NSG MHCI/II double knock out mice were i.v. implanted with luciferase-expressing C4-2 tumor cells, engrafted with human PBMCs from two different donors, and treated with one dose of 30D12-TED-CD3 control, 30D12-TED-K29/SN75 or 30D12-TED3-K29/SN75 V12 T cell engagers. Serum levels of TNFα were measured 6 hours after treatment. Mice treated with 30D12-TED-K29/SN75 or 30D12-TED3-K29/SN75 V12 demonstrated reduced TNFα levels compared to 30D12-TED-CD3 control treated mice (FIG. 9).

### Example 8—Tumor Cell/PBMC Admix Mouse Model

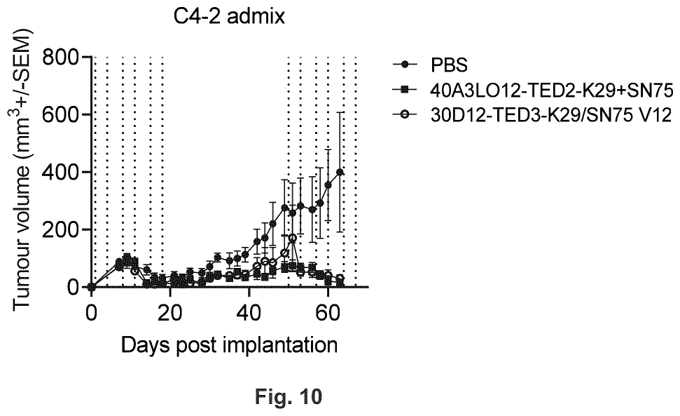

NSG MHCI/II double knock out mice were s.c. implanted with a mixture of C4-2 tumor cells and human PBMCs, and treated with 40A3LO12-TED2-K29/SN75 or 30D12-TED3-K29/SN75 T cell engagers. A reduction in tumor volume was observed upon treatment with 40A3LO12-TED2-K29/SN75 or 30D12-TED3-K29/SN75 molecules compared to PBS control mice (FIG. 10).

### Example 9—Human CD34+ Stem Cell Engrafted Mouse Model

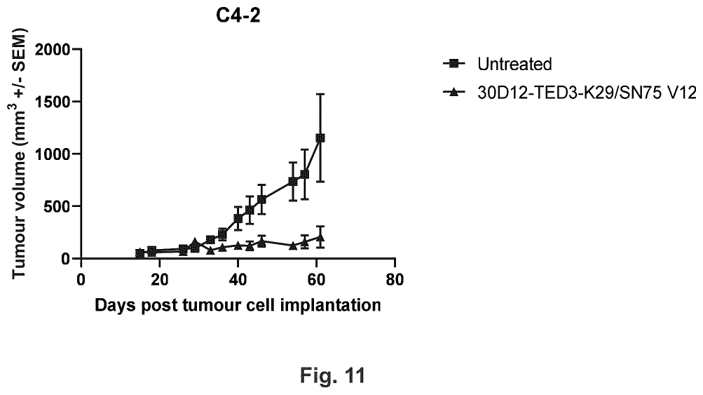

NSG mice were engrafted with human CD34+ stem cells and subsequently implanted s.c. with C4-2 tumor cells and treated with 30D12-TED3-K29/SN75 V12 T cell engager. Mice receiving 30D12-TED3-K29/SN75 V12 showed reduced tumor volumes compared to untreated control mice (FIG. 11).

### Example 10—Correlation Between Potency And STEAP2 Expression Level

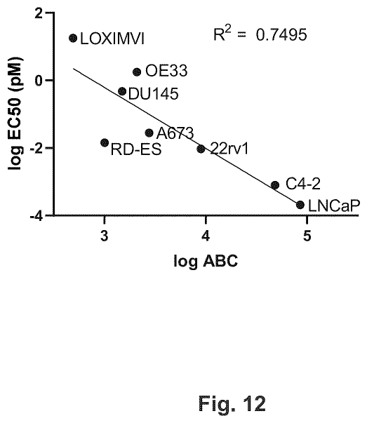

A positive correlation was observed between the potency of cytolysis after 3 days (expressed as EC50 of cytotoxicity) and the surface expression level of STEAP2 on tumor cells across 8 tumor cell lines (FIG. 12).

### Example 11—Impact of Regulatory T Cells on CD8 T Cell Activation

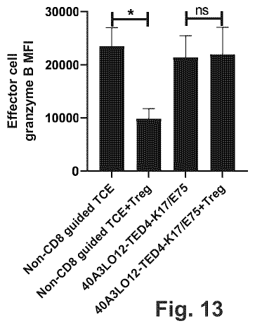

Addition of Tregs into co-cultures of CD3+ T cells and C4-2 tumor cells at a ratio of 1:4:0.8 reduced the level of CD8 T cell granzyme B production triggered in response to treatment for 3 days with 0.1 mg/kg of a non-CD8 guided T cell engaging molecule. In contrast, addition of Tregs did not impact the level of CD8 T cell granzyme B production triggered in response to treatment with 0.1 mg/kg of CD8-guided 40A3LO12-TED4-K17/E75 molecule (FIG. 13).

### Example 12—Serial Killing Assay

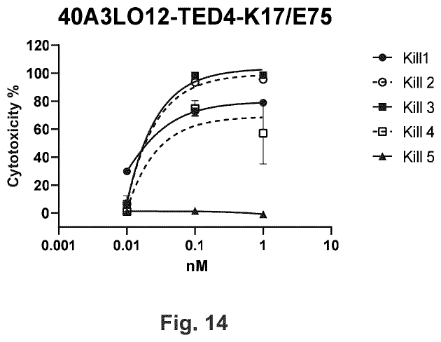

Cytotoxicity was measured at three concentrations of 40A3LO12-TED4-K17/E75 (0.01 nM, 0.1 nM, and 1 nM) across repeated rounds of co-culture with C4-2 tumor cells and sustained levels of cytotoxicity were observed for the first 4 rounds (FIG. 14).

### Example 13—Bystander Killing Assay

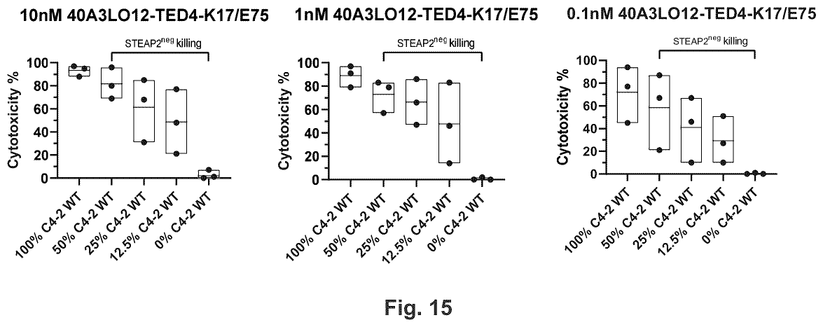

Although very little cytotoxicity of STEAP2-negative C4-2 cells alone by 40A3LO12-TED4-K17/E75 is seen (“0% C4-2 WT” condition), when these STEAP2-negative C4-2 cells are co-cultured with varying proportions of STEAP2-positive C4-2 WT cells (12.5%, 25%, 50%, or 100% C4-2 WT cells), killing of the STEAP2-negative C4-2 cells is detected even with as little as 12.5% of STEAP2-positive cells present (so-called bystander killing) (FIG. 15).

### Example 14—Prostate Cancer Organoid Assay

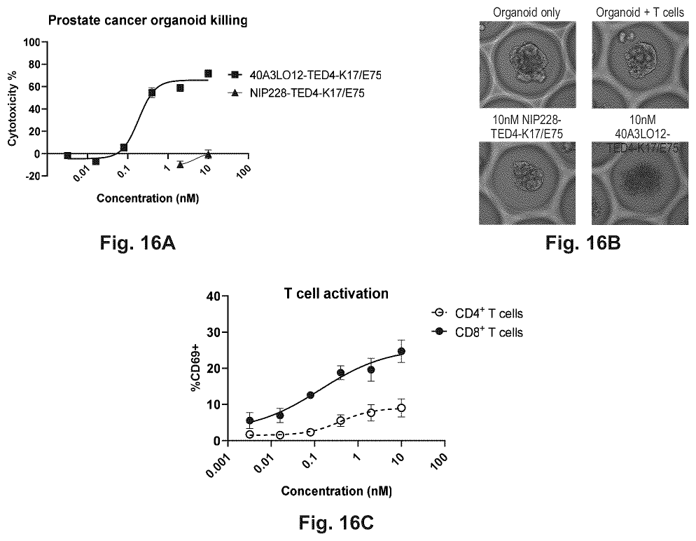

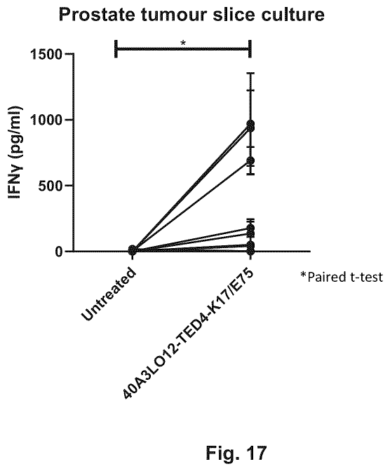

Organoids were generated from a prostate cancer patient-derived xenograft fragment and co-cultured with T cells. Treatment with 40A3LO12-TED4-K17/E75 induced cytotoxicity and preferential activation of CD8+ T cells over CD4+ T cells in this assay (FIG. 16).

### Example 15—Prostate Tumor Slice Assay

Treatment of primary prostate tumor slices with 40A3LO12-TED4-K17/E75 for 3 days induced increased release of IFNγ compared to untreated slices.

### Example 16—Preferential CD8+ T Cell Activation In Vivo

NSG MHCI/II double knock out mice were engrafted with human PBMCs, s.c. injected with 22Rv1 tumor cells, and treated with 0.1 mg/kg 40A3LO12-TED4-K17/E75. 24h after the third dose, blood and tumors were harvested and T cells were analysed by flow cytometry. Expression of T cell activation markers CD69 and CD25 was increased only on intratumoral CD8+ T cells but not on intratumoral CD4+ T cells or blood T cells (FIGS. 18B and 18C).

### Sequences

| SEQ | | |
|---|---|---|
| ID | | Sequence |
| 1 | STEAP2 | SYGMS |
| | 30D12 G96P | |
| | VH CDR1 | |
| 2 | STEAP2 | TISSGGSYTFYPDIMKG |
| | 30D12 G96P | |
| | VH CDR2 | |
| 3 | STEAP2 | RPYGTIYTFSFDA |
| | 30D12 G96P | |
| | VH CDR3 | |
| 4 | STEAP2 | RSSQSVVHSNANTYLE |
| | 30D12 G96P | |
| | VL CDR1 | |
| 5 | STEAP2 | KVSNRFS |
| | 30D12 G96P | |
| | VL CDR2 | |
| 6 | STEAP2 | FQGSHVPYT |
| | 30D12 G96P | |
| | VL CDR3 | |
| 7 | STEAP2 | EVQLVESGGGLVKPGGSLRLSCAASGFTFSSYGMSWVRQAPGKR |
| | 30D12 G96P | LEWVATISSGGSYTFYPDIMKGRFTISRDNSKNTLYLQMNSLRAE |
| | VH | DTAVYYCARRPYGTIYTFSFDAWGQGTTLTVSS |
| 8 | STEAP2 | DVVMTQSPLSLPVTLGQPASISCRSSQSVVHSNANTYLEWYLQKP |
| | 30D12 G96P | GQSPQLLIYKVSNRFSGVPDRFSGSGSGTDFTLKISRVEAEDVGV |
| | VL | YYCFQGSHVPYTFGQGTKLEIK |
| 9 | STEAP2-2 | SYGMS |
| | VH CDR1 | |
| 10 | STEAP2-2 | TISSGGSYTFYPDIMKG |
| | VH CDR2 | |
| 11 | STEAP2-2 | RPYGTIYTFSFDS |
| | VH CDR3 | |
| 12 | STEAP2-2 | RSSQSVVHSNGNTYLE |
| | VL CDR1 | |
| 13 | STEAP2-2 | KVSNRFS |
| | VL CDR2 | |
| 14 | STEAP2-2 | FQGSHVPYT |
| | VL CDR3 | |
| 15 | STEAP2-2 | EVQLVESGGDLVKPGGSLKLSCAASGFSFSSYGMSWVRQTPDKR |
| | G96P VH | LEWVATISSGGSYTFYPDIMKGRFTISRDNAMNTLYLQMSSLKSE |
| | | DSAMYYCARRPYGTIYTFSFDSWGQGTTLTVSS |
| 16 | STEAP2-2 | DVLMTQTPLSLPVSLGDQASISCRSSQSVVHSNGNTYLEWYLQKP |
| | VL | GQSPKLLIYKVSNRFSGVPDRFSGSGSGTDFTLKISRVEAEDLGVY |
| | | YCFQGSHVPYTFGGGTKLEIK |
| 17 | STEAP2-2 | SYGMS |
| | Humanized2 | |
| | VH CDR1 | |
| 18 | STEAP2-2 | TISSGGSYTFYPDIMKG |
| | Humanized2 | |
| | VH CDR2 | |
| 19 | STEAP2-2 | RPYGTIYTFSFDA |
| | Humanized2 | |
| | G96P VH | |
| | CDR3 | |
| 20 | STEAP2-2 | RSSQSVVHSNANTYLE |
| | Humanized2 | |
| | VL CDR1 | |
| 21 | STEAP2-2 | KVSNRFS |
| | Humanized2 | |
| | VL CDR2 | |
| 22 | STEAP2-2 | FQGSHVPYT |
| | Humanized2 | |
| | VL CDR3 | |
| 23 | STEAP2-2 | EVQLLESGGGLVQPGGSLRLSCAASGFTFSSYGMSWVRQAPGKR |
| | G96P VH | LEWVSTISSGGSYTFYPDIMKGRFTISRDNSKNTLYLQMNSLRAE |
| | | DTAVYYCARRPYGTIYTFSFDAWGQGTTLTVSS |
| 24 | STEAP2-2 | DVVMTQSPLSLPVTLGQPASISCRSSQSVVHSNANTYLEWYLQKP |
| | Humanized2 | GQSPQLLIYKVSNRFSGVPDRFSGSGSGTDFTLKISRVEAEDVGV |
| | VL | YYCFQGSHVPYTFGQGTKLEIK |
| 25 | 30D12-TED3 | DVVMTQSPLSLPVTLGQPASISCRSSQSVVHSNANTYLEWYLQKP |
| | Light Chain | GQSPQLLIYKVSNRFSGVPDRFSGSGSGTDFTLKISRVEAEDVGV |
| | | YYCFQGSHVPYTFGQGTKLEIKRTVAAPSVFIFPPSDEQLKSGTAS |
| | | VECLLNNFYPREAKVQWKVDNALQSGNSQESVTEQDSKDSTYS |
| | | LSSTLTLSKADYEKHKVYACEVTHQGLSSPVTKSFNRGEC |
| 26 | 30D12- | EVQLVESGGGLVKPGGSLRLSCAASGFTFSSYGMSWVRQAPGKR |
| | TED/TED3 | LEWVATISSGGSYTFYPDIMKGRFTISRDNSKNTLYLQMNSLRAE |
| | “hole” heavy | DTAVYYCARRPYGTIYTFSFDAWGQGTTLTVSSASTKGPSVFPLA |
| | chain | PSSKSTSGGTAALGCLVKDYFPEPVTVSWNSGALTSGVHTFPAVL |
| | | QSSGLYSLKSVVTVPSSSLGTQTYICNVNHKPSNTKVDKRVEPKS |
| | | CDKTHTCPPCPAPEFEGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPASIEKTISKAKGQPREPQVCTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQK |
| | | SLSLSPGK |
| 27 | CD3 Vκ17 | RSSQSLVHNTGNTYLS |
| | CDR1 | |
| 28 | CD3 Vκ17 | KVSNRAS |
| | CDR2 | |
| 29 | CD3 Vκ17 | GQGTQAPFT |
| | CDR3 | |
| 30 | Vκ17 | DIVMTQSPLSLPVTPGEPASISCRSSQSLVHNTGNTYLSWYLQKPG |
| | | QSPQSLIYKVSNRASGVPDRFSGSGSGTDFTLKISRVEAEDVGVY |
| | | YCGQGTQAPFTFGSGTKVEIK |
| 31 | CD3 Vκ29 | RSSQSLVHNTGNTYLS |
| | CDR1 | |
| 32 | CD3 Vκ29 | KVSNRAS |
| | CDR2 | |
| 33 | CD3 Vκ29 | GQGTQFPFT |
| | CDR3 | |
| 34 | Vκ29 | DIVMTQSPLSLPVTPGEPASISCRSSQSLVHNTGNTYLSWYLQKPG |
| | | QSPQSLIYKVSNRASGVPDRFSGSGSGTDFTLKISRVEAEDVGVY |
| | | YCGQGTQFPFTFGSGTKVEIK |
| 35 | TED3 CD3 | DIVMTQSPLSLPVTPGEPASISCRSSQSLVHNTGNTYLSWYLQKPG |
| | Light Chain | QSPQSLIYKVSNRASGVPDRESGSGSGTDFTLKISRVEAEDVGVY |
| | K29 | YCGQGTQFPFTFGSGTKVEIKGQPKAAPSVRLFPPCSEELQANKA |
| | | TLVCLISDFYPGAVTVAWKADSSPVKAGVETTTPSKQSNNKYAA |
| | | SSYLSLTPEQWKSHRSYSCQVTHEGSTVEKTVAPTEVS |
| 36 | CD3 VH | NAWMH |
| | CDR1 SN75 | |
| 37 | CD3 VH | QIKDRSQTYATYYAESVKGR |
| | CDR2 SN75 | |
| 38 | CD3 VH | RGVYYANAPFDY |
| | CDR3 SN75 | |
| 39 | CD3 VH | EVQLVESGGALVKPGGSLRLSCAASGFTFSNAWMHWVRQAPGK |
| | SN75 | QLEWVAQIKDRSQTYATYYAESVKGRFTISRDDSKNTLYLQMNS |
| | | LKTEDTAVYYCRGVYYANAPFDYWGQGTLVTVSS |
| 40 | CD3 VH | NAWMH |
| | CDR1 SN79 | |
| 41 | CD3 VH | QIKDRSQTYATYYAESVKGR |
| | CDR2 SN79 | |
| 42 | CD3 VH | RGVYYAVTPFDY |
| | CDR3 SN79 | |
| 43 | CD3 VH | EVQLVESGGALVKPGGSLRLSCAASGFTFSNAWMHWVRQAPGK |
| | SN79 | QLEWVAQIKDRSQTYATYYAESVKGRFTISRDDSKNTLYLQMNS |
| | | LKTEDTAVYYCRGVYYAVTPFDYWGQGTLVTVSS |
| 44 | CD3 VH | EAWMH |
| | CDR1 E75 | |
| 45 | CD3 VH | QIKDRSQTYATYYAESVKGR |
| | CDR2 E75 | |
| 46 | CD3 VH | RGVYYANAPFDY |
| | CDR3 E75 | |
| 47 | CD3 VH E75 | EVQLVESGGALVKPGGSLRLSCAASGFTFTEAWMHWVRQAPGK |
| | | QLEWVAQIKDRSQTYATYYAESVKGRFTISRDDSKNTLYLQMNS |
| | | LKTEDTAVYYCRGVYYANAPFDYWGQGTLVTVSS |
| 48 | CD8 VH | DFGMN |
| | CDR1 | |
| 49 | CD8 VH | LIYYDGSNKFYADSVKG |
| | CDR2 | |
| 50 | CD8 VH | PHYDGYYHFFDS |
| | CDR3 | |
| 51 | CD8 VL | KGSQDINNYLA |
| | CDR1 | |
| 52 | CD8 VL | NTDILHT |
| | CDR2 | |
| 53 | CD8 VL | YQYNNGYT |
| | CDR3 | |
| 54 | CD8 VL | DIQMTQSPSSLSASVGDRVTITCKGSQDINNYLAWYQQKPGKAP |
| | | KLLIYNTDILHTGVPSRFSGSGSGTDFTFTISSLQPEDIATYYCYQY |
| | | NNGYTFGQGTKVEIK |
| 55 | CD8 LC | DIQMTQSPSSLSASVGDRVTITCKGSQDINNYLAWYQQKPGKAP |
| | VL SEQ ID | KLLIYNTDILHTGVPSRFSGSGSGTDFTFTISSLQPEDIATYYCYQY |
| | NO: 54 + | NNGYTFGQGTKVEIKRTVAAPSVFIFPPCDEQLKSGTASVKCLLN |
| | mutant Cκ | NFYPREAKVQWKVDNALQSGNSQESVTEQDSKDSTYSLSSTLTL |
| | SEQ ID NO: | SKADYEKHKVYACEVTHQGLSSPVTKSFNRGEV |
| | 64 | |
| 56 | CD8 VH | QVQLVESGGGVVQPGRSLRLSCAASGFTFSDFGMNWVRQAPGK |
| | | GLEWVALIYYDGSNKFYADSVKGRFTISRDNSKNTLYLQMNSLR |
| | | AEDTAVYYCAKPHYDGYYHFFDSWGQGTLVTVSS |
| 57 | CD8 CH1 | ASTKGPSVCPLAPSSKSTSGGTAALGCLVKDYFPEPVTVSWNSGA |
| | CH1-portion | LTSGVHTFPAVLQSSGLYSLESVVTVPSSSLGTQTYICNVNHKPSN |
| | of SEQ ID | TKVDKRVEPKSVD |
| | NO: 78 w/ | |
| | S183E | |
| 58 | “knob” HC | QVQLVESGGGVVQPGRSLRLSCAASGFTFSDFGMNWVRQAPGK |
| | SEQ ID NO: | GLEWVALIYYDGSNKFYADSVKGRFTISRDNSKNTLYLQMNSLR |
| | 56 CD8 VH + | AEDTAVYYCAKPHYDGYYHFFDSWGQGTLVTVSSASTKGPSVC |
| | SEQ ID NO: | PLAPSSKSTSGGTAALGCLVKDYFPEPVTVSWNSGALTSGVHTFP |
| | 57 CD8 CH1 + | AVLQSSGLYSLESVVTVPSSSLGTQTYICNVNHKPSNTKVDKRVE |
| | SEQ ID | PKSVDGGGGSGGGGSEVQLVESGGALVKPGGSLRLSCAASGFTF |
| | NO: 89 | SNAWMHWVRQAPGKQLEWVAQIKDRSQTYATYYAESVKGRFTI |
| | linker + | SRDDSKNTLYLQMNSLKTEDTAVYYCRGVYYANAPFDYWGQG |
| | SEQ ID | TLVTVSSASTKGPSVCPLAPSSKSTSGGTADLGCLVKDYFPEPVT |
| | NO: 39 CD3 | VSWNSGALTSGVHTFPAVLQSSGLYSLSSVVTVPSSSLGTQTYIC |
| | VH SN75 + | NVNHKPSNTKVDKRVEPKSVDKTHTCPPCPAPEFEGGPSVFLFPP |
| | SEQ ID NO: | KPKDTLMISRTPEVTCVVVDVSHEDPEVKFNWYVDGVEVHNAK |
| | 79 IgG1 HC | TKPREEQYNSTYRVVSVLTVLHQDWLNGKEYKCKVSNKALPASI |
| | | EKTISKAKGQPREPQVYTLPPCREEMTKNQVSLWCLVKGFYPSDI |
| | | AVEWESNGQPENNYKTTPPVLDSDGSFFLYSKLTVDKSRWQQG |
| | | NVFSCSVMHEALHNHYTQKSLSLSPGK |
| 59 | wt Cλ | GQPKAAPSVTLFPPSSEELQANKATLVCLISDFYPGAVTVAWKAD |
| | | SSPVKAGVETTTPSKQSNNKYAASSYLSLTPEQWKSHRSYSCQVT |
| | | HEGSTVEKTVAPTECS |
| 60 | V12 Cλ | GQPKAAPSVTLFPPCSEELQANKATLVCLISDFYPGAVTVAWKA |
| | (S122C, | DSSPVKAGVETTTPSKQSNNKYAASSYLSLTPEQWKSHRSYSCQ |
| | C212V) | VTHEGSTVEKTVAPTEVS |
| 61 | V12Cλ of | GQPKAAPSVRLFPPCSEELQANKATLVCLISDFYPGAVTVAWKA |
| | SEQ ID NO: | DSSPVKAGVETTTPSKQSNNKYAA |
| | 60 w/ T117R | SSYLSLTPEQWKSHRSYSCQVTHEGSTVEKTVAPTEVS |
| 62 | wt Cκ | RTVAAPSVFIFPPSDEQLKSGTASVVCLLNNFYPREAKVQWKVD |
| | | NALQSGNSQESVTEQDSKDSTYSLSSTLTLSKADYEKHKVYACE |
| | | VTHQGLSSPVTKSFNRGEC |
| 63 | wt Cκ of SEQ | RTVAAPSVFIFPPSDEQLKSGTASVECLLNNFYPREAKVQWKVD |
| | ID NO: 62 w/ | NALQSGNSQESVTEQDSKDSTYSLSSTLTLSKADYEKHKVYACE |
| | V133E | VTHQGLSSPVTKSFNRGEC |
| 64 | mutant Cκ | RTVAAPSVFIFPPCDEQLKSGTASVKCLLNNFYPREAKVQWKVD |
| | S121C, | NALQSGNSQESVTEQDSKDSTYSLSSTLTLSKADYEKHKVYACE |
| | V133K, | VTHQGLSSPVTKSFNRGEV |
| | C214V | |
| 65 | IgG1 wt CH1 | ASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSN |
| | | TKVDKRVEPKSC |
| 66 | V12 CH1 | ASTKGPSVCPLAPSSKSTSGGTAALGCLVKDYFPEPVTVSWNSGA |
| | (F126C, | LTSGVHTFPAVLQSSGLYSLSSVVTVPSSSLGTQTYICNVNHKPSN |
| | C220V) | TKVDKRVEPKSV |
| 67 | wt CH1 of | ASTKGPSVFPLAPSSKSTSGGTAALGCLVKDYFPEPVTVSWNSGA |
| | SEQ ID NO: | LTSGVHTFPAVLQSSGLYSLKSVVTVPSSSLGTQTYICNVNHKPS |
| | 65 w/ S183K | NTKVDKRVEPKSC |
| 68 | IgG1 wt CH2 | LLGGPSVFLFPPKPKDTLMISRTPEVTCVVVDVSHEDPEVKFNWY |
| | | VDGVEVHNAKTKPREEQYNSTYRVVSVLTVLHQDWLNGKEYK |
| | | CKVSNKALPAPIEKTIS |
| 69 | wt CH2 of | FEGGPSVFLFPPKPKDTLMISRTPEVTCVVVDVSHEDPEVKFNWY |
| | SEQ ID NO: | VDGVEVHNAKTKPREEQYNSTYRVVSVLTVLHQDWLNGKEYK |
| | 68 w/ L234F, | CKVSNKALPASIEKTIS |
| | L235E, and | |
| | P331S | |
| 70 | IgG1wt CH3 | GQPREPQVYTLPPSREEMTKNQVSLTCLVKGFYPSDIAVEWESN |
| | | GQPENNYKTTPPVLDSDGSFFLYSKLTVDKSRWQQGNVFSCSVM |
| | | HEALHNHYTQKSLSLSPGK |
| 71 | IgG1 wt | ASTKGPSVFPLAPSSKSTSGGTAALGCLVKDYFPEPVTVSWNSGA |
| | constant | LTSGVHTFPAVLQSSGLYSLSSVVTVPSSSLGTQTYICNVNHKPSN |
| | domain HC | TKVDKRVEPKSCDKTHTCPPCPAPELLGGPSVFLFPPKPKDTLMIS |
| | | RTPEVTCVVVDVSHEDPEVKFNWYVDGVEVHNAKTKPREEQYN |
| | | STYRVVSVLTVLHQDWLNGKEYKCKVSNKALPAPIEKTISKAKG |
| | | QPREPQVYTLPPSREEMTKNQVSLTCLVKGFYPSDIAVEWESNG |
| | | QPENNYKTTPPVLDSDGSFFLYSKLTVDKSRWQQGNVFSCSVMH |
| | | EALHNHYTQKSLSLSPGK |
| 72 | IgG1 CH3 | GQPREPQVYTLPPSREEMTKNQVSLSCAVKGFYPSDIAVEWESN |
| | T366S, | GQPENNYKTTPPVLDSDGSFFLVSKLTVDKSRWQQGNVFSCSVM |
| | L368A, | HEALHNHYTQKSLSLSPGK |
| | Y407V | |
| | [hole] | |
| 73 | IgG1 CH3 | GQPREPQVCTLPPSREEMTKNQVSLSCAVKGFYPSDIAVEWESN |
| | Y349C | GQPENNYKTTPPVLDSDGSFFLVSKLTVDKSRWQQGNVFSCSVM |
| | T366S, | HEALHNHYTQKSLSLSPGK |
| | L368A, | |
| | Y407V | |
| 74 | IgG1 CH3 | GQPREPQVYTLPPSREEMTKNQVSLWCLVKGFYPSDIAVEWESN |
| | T366W | GQPENNYKTTPPVLDSDGSFFLYSKLTVDKSRWQQGNVFSCSVM |
| | [knob] | HEALHNHYTQKSLSLSPGK |
| 75 | IgG1 CH3 | GQPREPQVYTLPPCREEMTKNQVSLWCLVKGFYPSDIAVEWESN |
| | S354C, | GQPENNYKTTPPVLDSDGSFFLYSKLTVDKSRWQQGNVFSCSVM |
| | T366W | HEALHNHYTQKSLSLSPGK |
| 76 | IgG1 hole | ASTKGPSVFPLAPSSKSTSGGTAALGCLVKDYFPEPVTVSWNSGA |
| | T366S, | LTSGVHTFPAVLQSSGLYSLSSVVTVPSSSLGTQTYICNVNHKPSN |
| | L368A, | TKVDKRVEPKSCDKTHTCPPCPAPELLGGPSVFLFPPKPKDTLMIS |
| | Y407V, | RTPEVTCVVVDVSHEDPEVKFNWYVDGVEVHNAKTKPREEQYN |
| | Y349C | STYRVVSVLTVLHQDWLNGKEYKCKVSNKALPAPIEKTISKAKG |
| | | QPREPQVCTLPPSREEMTKNQVSLSCAVKGFYPSDIAVEWESNG |
| | | QPENNYKTTPPVLDSDGSFFLVSKLTVDKSRWQQGNVFSCSVMH |
| | | EALHNHYTQKSLSLSPGK |
| 77 | IgG1 HC | ASTKGPSVFPLAPSSKSTSGGTAALGCLVKDYFPEPVTVSWNSGA |
| | knob CH1, | LTSGVHTFPAVLQSSGLYSLSSVVTVPSSSLGTQTYICNVNHKPSN |
| | CH2, CH3 | TKVDKRVEPKSCDKTHTCPPCPAPELLGGPSVFLFPPKPKDTLMIS |
| | T366W, | RTPEVTCVVVDVSHEDPEVKFNWYVDGVEVHNAKTKPREEQYN |
| | S354C | STYRVVSVLTVLHQDWLNGKEYKCKVSNKALPAPIEKTISKAKG |
| | | QPREPQVYTLPPCREEMTKNQVSLWCLVKGFYPSDIAVEWESNG |
| | | QPENNYKTTPPVLDSDGSFFLYSKLTVDKSRWQQGNVFSCSVMH |
| | | EALHNHYTQKSLSLSPGK |
| 78 | “V12” with | ASTKGPSVCPLAPSSKSTSGGTAALGCLVKDYFPEPVTVSWNSGA |
| | stabilizing | LTSGVHTFPAVLQSSGLYSLSSVVTVPSSSLGTQTYICNVNHKPSN |
| | cysteine, | TKVDKRVEPKSVDKTHTCPPCPAPELLGGPSVFLFPPKPKDTLMIS |
| | interchain | RTPEVTCVVVDVSHEDPEVKFNWYVDGVEVHNAKTKPREEQYN |
| | cysteine | STYRVVSVLTVLHQDWLNGKEYKCKVSNKALPAPIEKTISKAKG |
| | mutations, | QPREPQVYTLPPCREEMTKNQVSLWCLVKGFYPSDIAVEWESNG |
| | and “knob” | QPENNYKTTPPVLDSDGSFFLYSKLTVDKSRWQQGNVFSCSVMH |
| | mutations: | EALHNHYTQKSLSLSPGK |
| | T366W, | |
| | F126C, | |
| | C220V, | |
| | S354C | |
| 79 | SEQ ID NO: | ASTKGPSVCPLAPSSKSTSGGTADLGCLVKDYFPEPVTVSWNSGA |
| | 78 w/ A141D, | LTSGVHTFPAVLQSSGLYSLSSVVTVPSSSLGTQTYICNVNHKPSN |
| | L234F, | TKVDKRVEPKSVDKTHTCPPCPAPEFEGGPSVFLFPPKPKDTLMIS |
| | L235E, | RTPEVTCVVVDVSHEDPEVKFNWYVDGVEVHNAKTKPREEQYN |
| | P331S | STYRVVSVLTVLHQDWLNGKEYKCKVSNKALPASIEKTISKAKG |
| | | QPREPQVYTLPPCREEMTKNQVSLWCLVKGFYPSDIAVEWESNG |
| | | QPENNYKTTPPVLDSDGSFFLYSKLTVDKSRWQQGNVFSCSVMH |
| | | EALHNHYTQKSLSLSPGK |
| 80 | IgG1 hole w/ | ASTKGPSVCPLAPSSKSTSGGTAALGCLVKDYFPEPVTVSWNSGA |
| | CH1 F126C, | LTSGVHTFPAVLQSSGLYSLSSVVTVPSSSLGTQTYICNVNHKPSN |
| | C220V + | TKVDKRVEPKSVDKTHTCPPCPAPELLGGPSVFLFPPKPKDTLMIS |
| | CH2/CH3 of | RTPEVTCVVVDVSHEDPEVKFNWYVDGVEVHNAKTKPREEQYN |
| | SEQ ID NO: | STYRVVSVLTVLHQDWLNGKEYKCKVSNKALPAPIEKTISKAKG |
| | 76 | QPREPQVCTLPPSREEMTKNQVSLSCAVKGFYPSDIAVEWESNG |
| | | QPENNYKTTPPVLDSDGSFFLVSKLTVDKSRWQQGNVFSCSVMH |
| | | EALHNHYTQKSLSLSPGK |
| | STEAP2 LC | SEQ ID NO: 25 |
| | SEQ ID NO: | |
| | 8 + SEQ ID | |
| | NO: 63 (wt | |
| | Cκ but | |
| | V133E) | |
| | CD3 LC | SEQ ID NO: 35 |
| 81 | STEAP2/CD3 | EVQLVESGGGLVKPGGSLRLSCAASGFTFSSYGMSWVRQAPGKR |
| | knob HC: | LEWVATISSGGSYTFYPDIMKGRFTISRDNSKNTLYLQMNSLRAE |
| | SEQ ID NO: | DTAVYYCARRPYGTIYTFSFDAWGQGTTLTVSSASTKGPSVFPLA |
| | 7 VH | PSSKSTSGGTAALGCLVKDYFPEPVTVSWNSGALTSGVHTFPAVL |
| | STEAP2 + | QSSGLYSLKSVVTVPSSSLGTQTYICNVNHKPSNTKVDKRVEPKS |
| | SEQ ID NO: | CDGGGGSGGGGSEVQLVESGGALVKPGGSLRLSCAASGFTFSNA |
| | 67 wt CH1 w/ | WMHWVRQAPGKQLEWVAQIKDRSQTYATYYAESVKGRFTISRD |
| | S183K + “D” + | DSKNTLYLQMNSLKTEDTAVYYCRGVYYANAPFDYWGQGTLV |
| | SEQ I DNO: 86 | TVSSASTKGPSVCPLAPSSKSTSGGTADLGCLVKDYFPEPVTVSW |
| | linker + | NSGALTSGVHTFPAVLQSSGLYSLSSVVTVPSSSLGTQTYICNVN |
| | SEQ ID | HKPSNTKVDKRVEPKSVDKTHTCPPCPAPPVAGPSVFLFPPKPKD |
| | NO: 39 CD3 | TLMISRTPEVTCVVVDVKHEDPEVKFNWYVDGVEVHNAKTKPR |
| | VH SN75 + | EEQYNSTYRVVSVLTVLHQDWLNGKEYKCKVSNKALPAPIEKTI |
| | SEQ ID NO: | SKAKGQPREPQVYTLPPCREEMTKNQVSLWCLVKGFYPSDIAVE |
| | 82. | WESNGQPENNYKTTPPVLDSDGSFFLYSKLTVDKSRWQQGNVFS |
| | | CSVMHEALHNHYTQKSLSLSPGK |
| 82 | CH1 w/ | ASTKGPSVCPLAPSSKSTSGGTADLGCLVKDYFPEPVTVSWNSGA |
| | A141D, CH2, | LTSGVHTFPAVLQSSGLYSLSSVVTVPSSSLGTQTYICNVNHKPSN |
| | being IgG2 at | TKVDKRVEPKSVDKTHTCPPCPAPPVAGPSVFLFPPKPKDTLMIS |
| | 233-236, | RTPEVTCVVVDVKHEDPEVKFNWYVDGVEVHNAKTKPREEQY |
| | S267K, and | NSTYRVVSVLTVLHQDWLNGKEYKCKVSNKALPAPIEKTISKAK |
| | wt P at 331; | GQPREPQVYTLPPCREEMTKNQVSLWCLVKGFYPSDIAVEWESN |
| | CH3 S354C, | GQPENNYKTTPPVLDSDGSFFLYSKLTVDKSRWQQGNVFSCSVM |
| | T366W. | HEALHNHYTQKSLSLSPGK |
| 83 | CD8 VHH | EVQLLESGGGLVQPGGSLRLSCAASGFTFDDYAIGWFRQAPGKE |
| | | REGVSCIRVSDGSTYYADSVKGRFTISRDNSKNTLYLQMNSLRAE |
| | | DTAVYYCAAGSLYTCVQSIVWPARPYYDMDYWGQGTLVTVSS |
| 84 | CD8 VHH | DDYAIG |
| | CDR1 | |
| 85 | CD8 VHH | CIRVSDGSTYYADSVKG |
| | CDR2 | |
| 86 | CD8 VHH | GSLYTCVQSIVWPARPYYDMDY |
| | CDR3 | |
| 87 | hole CD8 | EVQLLESGGGLVQPGGSLRLSCAASGFTFDDYAIGWFRQAPGKE |
| | VHH | REGVSCIRVSDGSTYYADSVKGRFTISRDNSKNTLYLQMNSLRAE |
| | STEAP2 HC: | DTAVYYCAAGSLYTCVQSIVWPARPYYDMDYWGQGTLVTVSSG |
| | SEQ ID NO: | GGGSGGGGSEVQLVESGGGLVKPGGSLRLSCAASGFTFSSYGMS |
| | 83 CD8 VHH + | WVRQAPGKRLEWVATISSGGSYTFYPDIMKGRFTISRDNSKNTLY |
| | SEQ ID | LQMNSLRAEDTAVYYCARRPYGTIYTFSFDAWGQGTTLTVSSAS |
| | NO: 89 | TKGPSVFPLAPSSKSTSGGTAALGCLVKDYFPEPVTVSWNSGALT |
| | linker + | SGVHTFPAVLQSSGLYSLKSVVTVPSSSLGTQTYICNVNHKPSNT |
| | SEQ ID NO: | KVDKRVEPKSCDKTHTCPPCPAPPVAGPSVFLFPPKPKDTLMISRT |
| | 7 STEAP2 | PEVTCVVVDVKHEDPEVKFNWYVDGVEVHNAKTKPREEQYNST |
| | VH + SEQ ID | YRVVSVLTVLHQDWLNGKEYKCKVSNKALPAPIEKTISKAKGQP |
| | NO: 88 | REPQVCTLPPSREEMTKNQVSLSCAVKGFYPSDIAVEWESNGQPE |
| | | NNYKTTPPVLDSDGSFFLVSKLTVDKSRWQQGNVFSCSVMHEAL |
| | | HNHYTQKSLSLSPGK |
| 88 | IgG1 hole of | ASTKGPSVFPLAPSSKSTSGGTAALGCLVKDYFPEPVTVSWNSGA |
| | SEQ ID NO: | LTSGVHTFPAVLQSSGLYSLKSVVTVPSSSLGTQTYICNVNHKPS |
| | 76 w/ S183K, | NTKVDKRVEPKSCDKTHTCPPCPAPPVAGPSVFLFPPKPKDTLMI |
| | CH2 being | SRTPEVTCVVVDVKHEDPEVKFNWYVDGVEVHNAKTKPREEQY |
| | IgG2 at 233- | NSTYRVVSVLTVLHQDWLNGKEYKCKVSNKALPAPIEKTISKAK |
| | 236, and | GQPREPQVCTLPPSREEMTKNQVSLSCAVKGFYPSDIAVEWESNG |
| | S267K | QPENNYKTTPPVLDSDGSFFLVSKLTVDKSRWQQGNVFSCSVMH |
| | | EALHNHYTQKSLSLSPGK |
| 89 | linker | GGGGS |
| 90 | STEAP2 VH + | EVQLVESGGGLVKPGGSLRLSCAASGFTFSSYGMSWVRQAPGKR |
| | CH1: | LEWVATISSGGSYTFYPDIMKGRFTISRDNSKNTLYLQMNSLRAE |
| | SEQ ID NO: | DTAVYYCARRPYGTIYTFSFDAWGQGTTLTVSSASTKGPSVEPLA |
| | 7 + SEQ ID | PSSKSTSGGTAALGCLVKDYFPEPVTVSWNSGALTSGVHTFPAVL |
| | NO: 67 | QSSGLYSLKSVVTVPSSSLGTQTYICNVNHKPSNTKVDKRVEPKS |
| | | C |
| 91 | STEAP2 hole | ASTKGPSVFPLAPSSKSTSGGTAALGCLVKDYFPEPVTVSWNSGA |
| | CH1 SEQ ID | LTSGVHTFPAVLQSSGLYSLKSVVTVPSSSLGTQTYICNVNHKPS |
| | NO: 67, CH2 | NTKVDKRVEPKSCDKTHTCPPCPAPEFEGGPSVFLFPPKPKDTLM |
| | SEQ ID NO: | ISRTPEVTCVVVDVSHEDPEVKFNWYVDGVEVHNAKTKPREEQY |
| | 69, CH3 SEQ | NSTYRVVSVLTVLHQDWLNGKEYKCKVSNKALPASIEKTISKAK |
| | ID NO: 73 | GQPREPQVCTLPPSREEMTKNQVSLSCAVKGFYPSDIAVEWESN |
| | | GQPENNYKTTPPVLDSDGSFFLVSKLTVDKSRWQQGNVFSCSVM |
| | | HEALHNHYTQKSLSLSPGK |
| 92 | “knob” HC | EVQLVESGGALVKPGGSLRLSCAASGFTFSNAWMHWVRQAPGK |
| | for TED SEQ | QLEWVAQIKDRSQTYATYYAESVKGRFTISRDDSKNTLYLQMNS |
| | ID NO: 39 | LKTEDTAVYYCRGVYYANAPFDYWGQGTLVTVSSASTKGPSVC |
| | CD3 VH | PLAPSSKSTSGGTADLGCLVKDYFPEPVTVSWNSGALTSGVHTFP |
| | SN75 + SEQ | AVLQSSGLYSLSSVVTVPSSSLGTQTYICNVNHKPSNTKVDKRVE |
| | ID NO: 79 | PKSVDKTHTCPPCPAPEFEGGPSVFLFPPKPKDTLMISRTPEVTCV |
| | IgG1 HC | VVDVSHEDPEVKFNWYVDGVEVHNAKTKPREEQYNSTYRVVSV |
| | | LTVLHQDWLNGKEYKCKVSNKALPASIEKTISKAKGQPREPQVY |
| | | TLPPCREEMTKNQVSLWCLVKGFYPSDIAVEWESNGQPENNYKT |
| | | TPPVLDSDGSFFLYSKLTVDKSRWQQGNVFSCSVMHEALHNHYT |
| | | QKSLSLSPGK |
| 93 | hole STEAP2 | EVQLVESGGGLVKPGGSLRLSCAASGFTFSSYGMSWVRQAPGKR |
| | HC for TED2: | LEWVATISSGGSYTFYPDIMKGRFTISRDNSKNTLYLQMNSLRAE |
| | SEQ ID NO: | DTAVYYCARRPYGTIYTFSFDAWGQGTTLTVSSASTKGPSVFPLA |
| | 7 STEAP2 | PSSKSTSGGTAALGCLVKDYFPEPVTVSWNSGALTSGVHTFPAVL |
| | VH + SEQ ID | QSSGLYSLKSVVTVPSSSLGTQTYICNVNHKPSNTKVDKRVEPKS |
| | NO: 88 | CDKTHTCPPCPAPPVAGPSVFLFPPKPKDTLMISRTPEVTCVVVD |
| | | VKHEDPEVKFNWYVDGVEVHNAKTKPREEQYNSTYRVVSVLTV |
| | | LHQDWLNGKEYKCKVSNKALPAPIEKTISKAKGQPREPQVCTLPP |
| | | SREEMTKNQVSLSCAVKGFYPSDIAVEWESNGQPENNYKTTPPV |
| | | LDSDGSFFLVSKLTVDKSRWQQGNVFSCSVMHEALHNHYTQKS |
| | | LSLSPGK |
| 94 | STEAP2-2 | RGYGTIYTFSFDA |
| | Humanized1 | |
| | VH CDR3 | |
| 95 | STEAP2-2 | EVQLVESGGGLVKPGGSLRLSCAASGFTFSSYGMSWVRQAPGKR |
| | Humanized1 | LEWVATISSGGSYTFYPDIMKGRFTISRDNSKNTLYLQMNSLRAE |
| | VH | DTAVYYCARRGYGTIYTFSFDAWGQGTTLTVSS |
| 96 | STEAP2-2 | RGYGTIYTFSFDS |
| | VH CDR3 | |
| 97 | STEAP2-2 | EVQLVESGGDLVKPGGSLKLSCAASGFSFSSYGMSWVRQTPDKR |
| | VH | LEWVATISSGGSYTFYPDIMKGRFTISRDNAMNTLYLQMSSLKSE |
| | | DSAMYYCARRGYGTIYTFSFDSWGQGTTLTVSS |
| 98 | STEAP2-2 | RGYGTIYTFSFDA |
| | Humanized2 | |
| | VH CDR3 | |
| 99 | STEAP2-2 | EVQLLESGGGLVQPGGSLRLSCAASGFTFSSYGMSWVRQAPGKR |
| | Humanized2 | LEWVSTISSGGSYTFYPDIMKGRFTISRDNSKNTLYLQMNSLRAE |
| | VH | DTAVYYCARRGYGTIYTFSFDAWGQGTTLTVSS |
| 100 | STEAP2-8 | RASQSVASNLA |
| | VL CDR1 | |
| 101 | STEAP2-8 | GASTRAT |
| | VL CDR2 | |
| 102 | STEAP2-8 | QQYNNWPFT |
| | VL CDR3 | |
| 103 | STEAP2-8 | RNSAVWN |
| | VH CDR1 | |
| 104 | STEAP2-8 | RTYYRSKWYNDYAPSVKS |
| | VH CDR2 | |
| 105 | STEAP2-8 | GLRQNQFYYYMDV |
| | VH CDR3 | |
| 106 | STEAP2-8 | QVQLQQSGPGLVKPSQTLSLTCAISGDSVSRNSAVWNWIRQSPSR |
| | VH | GLEWLGRTYYRSKWYNDYAPSVKSRITINPDTSKNQFSLQLNSV |
| | | TPEDTAVYYCARGLRQNQFYYYMDVWGKGTTVTVSS |
| 107 | STEAP2-8 | EIVMTQSPATLSVSPGERATLSCRASQSVASNLAWYQQKPGQAP |
| | VL | RLLIYGASTRATGIPARFSGSGSGTEFTLTISSLQSEDFAVYYCQQ |
| | | YNNWPFTFGPGTKVDIK |
| 108 | STEAP2 | RASQSVNSNLA |
| | 40A3 VL | |
| | CDR1 | |
| 109 | STEAP2 | GASTRAT |
| | 40A3 VL | |
| | CDR2 | |
| 110 | STEAP2 | QQYNNWPFT |
| | 40A3 VL | |
| | CDR3 | |
| 111 | STEAP2 | RNSAVWN |
| | 40A3 VH | |
| | CDR1 | |
| 112 | STEAP2 | RTYYRSKWYNDYAVSVKS |
| | 40A3 VH | |
| | CDR2 | |
| 113 | STEAP2 | GLLQNNFYYYMDV |
| | 40A3 VH | |
| | CDR3 | |
| 114 | STEAP2 | QVQLQQSGPGLVKPSQTLSLTCAISGDSVSRNSAVWNWIRQSPSR |
| | 40A3 VH | GLEWLGRTYYRSKWYNDYAVSVKSRITINPDTSKNQFSLQVNSV |
| | | TPEDTAVYYCARGLLQNNFYYYMDVWGKGTTVTVSS |
| 115 | STEAP2 | EIVMTQSPATLSVSPGERATLSCRASQSVNSNLAWYQQKPGQAP |
| | 40A3 VL | RLLIYGASTRATGIPARFSGSGSGTEFTLTISSLQSEDFAVYYCQQ |
| | | YNNWPFTFGPGTKVDIK |
| 116 | STEAP2 LC: | DVLMTQTPLSLPVSLGDQASISCRSSQSVVHSNGNTYLEWYLQKP |
| | SEQ ID NO: | GQSPKLLIYKVSNRFSGVPDRFSGSGSGTDFTLKISRVEAEDLGVY |
| | 16 + SEQ ID | YCFQGSHVPYTFGGGTKLEIK |
| | NO: 63 [wt | RTVAAPSVFIFPPSDEQLKSGTASVECLLNNFYPREAKVQWKVD |
| | Cκ w/ V133E] | NALQSGNSQESVTEQDSKDSTYSLSSTLTLSKADYEKHKVYACE |
| | | VTHQGLSSPVTKSFNRGEC |
| 117 | STEAP2 LC: | DVVMTQSPLSLPVTLGQPASISCRSSQSVVHSNANTYLEWYLQKP |
| | SEQ ID NO: | GQSPQLLIYKVSNRFSGVPDRFSGSGSGTDFTLKISRVEAEDVGV |
| | 24 + SEQ ID | YYCFQGSHVPYTFGQGTKLEIK |
| | NO: 63 [wt | RTVAAPSVFIFPPSDEQLKSGTASVECLLNNFYPREAKVQWKVD |
| | Cκ w/ V133E] | NALQSGNSQESVTEQDSKDSTYSLSSTLTLSKADYEKHKVYACE |
| | | VTHQGLSSPVTKSFNRGEC |
| 118 | STEAP2 LC: | EIVMTQSPATLSVSPGERATLSCRASQSVASNLAWYQQKPGQAP |
| | SEQ ID NO: | RLLIYGASTRATGIPARFSGSGSGTEFTLTISSLQSEDFAVYYCQQ |
| | 107 + SEQ ID | YNNWPFTFGPGTKVDIK |
| | NO: 63 [wt | RTVAAPSVFIFPPSDEQLKSGTASVECLLNNFYPREAKVQWKVD |
| | Cκ] w/ | NALQSGNSQESVTEQDSKDSTYSLSSTLTLSKADYEKHKVYACE |
| | V133E] | VTHQGLSSPVTKSFNRGEC |
| 119 | STEAP2 LC: | EIVMTQSPATLSVSPGERATLSCRASQSVNSNLAWYQQKPGQAP |
| | SEQ ID NO: | RLLIYGASTRATGIPARFSGSGSGTEFTLTISSLQSEDFAVYYCQQ |
| | 115 + SEQ ID | YNNWPFTFGPGTKVDIK |
| | NO: 63 [wt | RTVAAPSVFIFPPSDEQLKSGTASVECLLNNFYPREAKVQWKVD |
| | Cκ w/ V133E] | NALQSGNSQESVTEQDSKDSTYSLSSTLTLSKADYEKHKVYACE |
| | | VTHQGLSSPVTKSFNRGEC |
| 120 | STEAP2 HC | EVQLVESGGDLVKPGGSLKLSCAASGFSFSSYGMSWVRQTPDKR |
| | “hole”: | LEWVATISSGGSYTFYPDIMKGRFTISRDNAMNTLYLQMSSLKSE |
| | STEAP2 | DSAMYYCARRPYGTIYTFSFDSWGQGTTLTVSS |
| | VH SEQ ID | ASTKGPSVFPLAPSSKSTSGGTAALGCLVKDYFPEPVTVSWNSGA |
| | NO: 15 + | LTSGVHTFPAVLQSSGLYSLKSVVTVPSSSLGTQTYICNVNHKPS |
| | SEQ ID NO: | NTKVDKRVEPKSCDKTHTCPPCPAPEFEGGPSVFLFPPKPKDTLM |
| | 67 wt CH1 w/ | ISRTPEVTCVVVDVSHEDPEVKFNWYVDGVEVHNAKTKPREEQY |
| | S183K + SEQ | NSTYRVVSVLTVLHQDWLNGKEYKCKVSNKALPASIEKTISKAK |
| | ID NO: 69 + | GQPREPQVCTLPPSREEMTKNQVSLSCAVKGFYPSDIAVEWESN |
| | SEQ ID NO: | GQPENNYKTTPPVLDSDGSFFLVSKLTVDKSRWQQGNVFSCSVM |
| | 73 | HEALHNHYTQKSLSLSPGK |
| 121 | STEAP2 HC | EVQLLESGGGLVQPGGSLRLSCAASGFTFSSYGMSWVRQAPGKR |
| | “hole”: | LEWVSTISSGGSYTFYPDIMKGRFTISRDNSKNTLYLQMNSLRAE |
| | STEAP2 VH | DTAVYYCARRPYGTIYTFSFDAWGQGTTLTVSS |
| | SEQ ID NO: | ASTKGPSVFPLAPSSKSTSGGTAALGCLVKDYFPEPVTVSWNSGA |
| | 23 + SEQ ID | LTSGVHTFPAVLQSSGLYSLKSVVTVPSSSLGTQTYICNVNHKPS |
| | NO: 67 wt | NTKVDKRVEPKSCDKTHTCPPCPAPEFEGGPSVFLFPPKPKDTLM |
| | CH1 w/ | ISRTPEVTCVVVDVSHEDPEVKFNWYVDGVEVHNAKTKPREEQY |
| | S183K + SEQ | NSTYRVVSVLTVLHQDWLNGKEYKCKVSNKALPASIEKTISKAK |
| | ID NO: 69 + | GQPREPQVCTLPPSREEMTKNQVSLSCAVKGFYPSDIAVEWESN |
| | SEQ ID NO: | GQPENNYKTTPPVLDSDGSFFLVSKLTVDKSRWQQGNVFSCSVM |
| | 73 | HEALHNHYTQKSLSLSPGK |
| 122 | STEAP2 HC | EVQLVESGGGLVKPGGSLRLSCAASGFTFSSYGMSWVRQAPGKR |
| | “hole”: | LEWVATISSGGSYTFYPDIMKGRFTISRDNSKNTLYLQMNSLRAE |
| | STEAP2 VH | DTAVYYCARRGYGTIYTFSFDAWGQGTTLTVSS |
| | SEQ ID NO: | ASTKGPSVFPLAPSSKSTSGGTAALGCLVKDYFPEPVTVSWNSGA |
| | 95 + SEQ ID | LTSGVHTFPAVLQSSGLYSLKSVVTVPSSSLGTQTYICNVNHKPS |
| | NO: 67 wt | NTKVDKRVEPKSCDKTHTCPPCPAPEFEGGPSVFLFPPKPKDTLM |
| | CH1 w/ | ISRTPEVTCVVVDVSHEDPEVKFNWYVDGVEVHNAKTKPREEQY |
| | S183K + SEQ | NSTYRVVSVLTVLHQDWLNGKEYKCKVSNKALPASIEKTISKAK |
| | ID NO: 69 + | GQPREPQVCTLPPSREEMTKNQVSLSCAVKGFYPSDIAVEWESN |
| | SEQ ID NO: | GQPENNYKTTPPVLDSDGSFFLVSKLTVDKSRWQQGNVFSCSVM |
| | 73 | HEALHNHYTQKSLSLSPGK |
| 123 | STEAP2 HC | EVQLVESGGDLVKPGGSLKLSCAASGFSFSSYGMSWVRQTPDKR |
| | “hole”: | LEWVATISSGGSYTFYPDIMKGRFTISRDNAMNTLYLQMSSLKSE |
| | STEAP2 VH | DSAMYYCARRGYGTIYTFSFDSWGQGTTLTVSS |
| | SEQ ID NO: | ASTKGPSVFPLAPSSKSTSGGTAALGCLVKDYFPEPVTVSWNSGA |
| | 97 + SEQ ID | LTSGVHTFPAVLQSSGLYSLKSVVTVPSSSLGTQTYICNVNHKPS |
| | NO: 67 + | NTKVDKRVEPKSCDKTHTCPPCPAPEFEGGPSVFLFPPKPKDTLM |
| | SEQ ID NO: | ISRTPEVTCVVVDVSHEDPEVKFNWYVDGVEVHNAKTKPREEQY |
| | 69 + SEQ ID | NSTYRVVSVLTVLHQDWLNGKEYKCKVSNKALPASIEKTISKAK |
| | NO: 73 | GQPREPQVCTLPPSREEMTKNQVSLSCAVKGFYPSDIAVEWESN |
| | | GQPENNYKTTPPVLDSDGSFFLVSKLTVDKSRWQQGNVFSCSVM |
| | | HEALHNHYTQKSLSLSPGK |
| 124 | STEAP2 HC | EVQLLESGGGLVQPGGSLRLSCAASGFTFSSYGMSWVRQAPGKR |
| | “hole”: | LEWVSTISSGGSYTFYPDIMKGRFTISRDNSKNTLYLQMNSLRAE |
| | STEAP2 VH | DTAVYYCARRGYGTIYTFSFDAWGQGTTLTVSS |
| | SEQ ID NO: | ASTKGPSVFPLAPSSKSTSGGTAALGCLVKDYFPEPVTVSWNSGA |
| | 99 + SEQ ID | LTSGVHTFPAVLQSSGLYSLKSVVTVPSSSLGTQTYICNVNHKPS |
| | NO: 67 + | NTKVDKRVEPKSCDKTHTCPPCPAPEFEGGPSVFLFPPKPKDTLM |
| | SEQ ID NO: | ISRTPEVTCVVVDVSHEDPEVKFNWYVDGVEVHNAKTKPREEQY |
| | 69 + SEQ ID | NSTYRVVSVLTVLHQDWLNGKEYKCKVSNKALPASIEKTISKAK |
| | NO: 73 | GQPREPQVCTLPPSREEMTKNQVSLSCAVKGFYPSDIAVEWESN |
| | | GQPENNYKTTPPVLDSDGSFFLVSKLTVDKSRWQQGNVFSCSVM |
| | | HEALHNHYTQKSLSLSPGK |
| 125 | STEAP2 HC | QVQLQQSGPGLVKPSQTLSLTCAISGDSVSRNSAVWNWIRQSPSR |
| | “hole”: | GLEWLGRTYYRSKWYNDYAPSVKSRITINPDTSKNQFSLQLNSV |
| | STEAP2 VH | TPEDTAVYYCARGLRQNQFYYYMDVWGKGTTVTVSS |
| | SEQ ID NO: | ASTKGPSVFPLAPSSKSTSGGTAALGCLVKDYFPEPVTVSWNSGA |
| | 106 + SEQ ID | LTSGVHTFPAVLQSSGLYSLKSVVTVPSSSLGTQTYICNVNHKPS |
| | NO: 67 + | NTKVDKRVEPKSCDKTHTCPPCPAPEFEGGPSVFLFPPKPKDTLM |
| | SEQ ID NO: | ISRTPEVTCVVVDVSHEDPEVKFNWYVDGVEVHNAKTKPREEQY |
| | 69 + SEQ ID | NSTYRVVSVLTVLHQDWLNGKEYKCKVSNKALPASIEKTISKAK |
| | NO: 73 | GQPREPQVCTLPPSREEMTKNQVSLSCAVKGFYPSDIAVEWESN |
| | | GQPENNYKTTPPVLDSDGSFFLVSKLTVDKSRWQQGNVFSCSVM |
| | | HEALHNHYTQKSLSLSPGK |
| 126 | STEAP2 HC | QVQLQQSGPGLVKPSQTLSLTCAISGDSVSRNSAVWNWIRQSPSR |
| | “hole”: | GLEWLGRTYYRSKWYNDYAVSVKSRITINPDTSKNQFSLQVNSV |
| | STEAP2 VH | TPEDTAVYYCARGLLQNNFYYYMDVWGKGTTVTVSS |
| | SEQ ID NO: | ASTKGPSVFPLAPSSKSTSGGTAALGCLVKDYFPEPVTVSWNSGA |
| | 114 + SEQ ID | LTSGVHTFPAVLQSSGLYSLKSVVTVPSSSLGTQTYICNVNHKPS |
| | NO: 67 + | NTKVDKRVEPKSCDKTHTCPPCPAPEFEGGPSVFLFPPKPKDTLM |
| | SEQ ID NO: | ISRTPEVTCVVVDVSHEDPEVKFNWYVDGVEVHNAKTKPREEQY |
| | 69 + SEQ ID | NSTYRVVSVLTVLHQDWLNGKEYKCKVSNKALPASIEKTISKAK |
| | NO: 73 | GQPREPQVCTLPPSREEMTKNQVSLSCAVKGFYPSDIAVEWESN |
| | | GQPENNYKTTPPVLDSDGSFFLVSKLTVDKSRWQQGNVFSCSVM |
| | | HEALHNHYTQKSLSLSPGK |
| 127 | STEAP2-3 | RNSAVWN |
| | VH CDR1 | |
| 128 | STEAP2-3 | RTYYRSKWYNDYAVSVKS |
| | VH CDR2 | |
| 129 | STEAP2-3 | GLLQNQFYYYMDV |
| | VH CDR3 | |
| 130 | STEAP2-3 | RASQSVSSNLA |
| | VL | |
| | CDR1(40A3G | |
| | L-LO7) | |
| 131 | STEAP2-3 | GASTRAT |
| | VL CDR2 | |
| 132 | STEAP2-3 | QQYNNWPFT |
| | VL CDR3 | |
| 133 | STEAP2-3 | QVQLQQSGPGLVKPSQTLSLTCAISGDSVSRNSAVWNWIRQSPSR |
| | VH | GLEWLGRTYYRSKWYNDYAVSVKSRITINPDTSKNQFSLQLNSV |
| | | TPEDTAVYYCARGLLQNQFYYYMDVWGKGTTVTVSS |
| 134 | STEAP2-3 | EIVMTQSPATLSVSPGERATLSCRASQSVSSNLAWYQQKPGQAPR |
| | VL | LLIYGASTRATGIPARFSGSGSGTEFTLTISSLQSEDFAVYYCQQY |
| | | NNWPFTFGPGTKVDIK |
| 135 | 40A3GL- | RNSAVWN |
| | LO11 VH | |
| | CDR 1 | |
| 136 | 40A3GL- | RTYYRSKWYNDYAPSVKS |
| | LO11 VH | |
| | CDR 2 | |
| 137 | 40A3GL- | GLLQNQFYYYMDV |
| | LO11 VH | |
| | CDR 3 | |
| 138 | 40A3GL- | RASQSVASNLA |
| | LO11 VL | |
| | CDR 1 | |
| 139 | 40A3GL- | GASTRAT |
| | LO11 VL | |
| | CDR 2 | |
| 140 | 40A3GL- | QQYNNWPFT |
| | LO11 VL | |
| | CDR 3 | |
| 141 | 40A3GL- | QVQLQQSGPGLVKPSQTLSLTCAISGDSVSRNSAVWNWIRQSPSR |
| | LO11 VH | GLEWLGRTYYRSKWYNDYAPSVKSRITINPDTSKNQFSLQLNSV |
| | | TPEDTAVYYCARGLLQNQFYYYMDVWGKGTTVTVSS |
| 142 | 40A3GL- | EIVMTQSPATLSVSPGERATLSCRASQSVASNLAWYQQKPGQAP |
| | LO11 VL | RLLIYGASTRATGIPARFSGSGSGTEFTLTISSLQSEDFAVYYCQQ |
| | | YNNWPFTFGPGTKVDIK |
| 143 | STEAP2 | RNSAVWN |
| | 40A3 LO12 | |
| | VH CDR1 | |
| 144 | STEAP2 | RTYYRSKWYNDYAVSVKS |
| | 40A3 LO12 | |
| | VH CDR2 | |
| 145 | STEAP2 | GLRQNQFYYYMDV |
| | 40A3 LO12 | |
| | VH CDR3 | |
| 146 | STEAP240A3 | RASQSVASNLA |
| | LO12 VL | |
| | CDR1 | |
| 147 | STEAP2ª40A3 | GASTRAT |
| | LO12 VL | |
| | CDR2 | |
| 148 | STEAP2ª40A3 | QQYNNWPFT |
| | LO12 VL | |
| | CDR3 | |
| 149 | STEAP2 VH | QVQLQQSGPGLVKPSQTLSLTCAISGDSVSRNSAVWNWIRQSPSR |
| | 40A3 LO12 | GLEWLGRTYYRSKWYNDYAVSVKSRITINPDTSKNQFSLQLNSV |
| | | TPEDTAVYYCARGLRQNQFYYYMDVWGKGTTVTVSS |
| 150 | STEAP2 VL | EIVMTQSPATLSVSPGERATLSCRASQSVASNLAWYQQKPGQAP |
| | 40A3 LO12 | RLLIYGASTRATGIPARFSGSGSGTEFTLTISSLQSEDFAVYYCQQ |
| | | YNNWPFTFGPGTKVDIK |
| 151 | TED4 LO12 | QVQLQQSGPGLVKPSQTLSLTCAISGDSVSRNSAVWNWIRQSPSR |
| | “Knob” | GLEWLGRTYYRSKWYNDYAVSVKSRITINPDTSKNQFSLQLNSV |
| | Heavy Chain | TPEDTAVYYCARGLRQNQFYYYMDVWGKGTTVTVSSASTKGPS |
| | | VFPLAPSSKSTSGGTAALGCLVKDYFPEPVTVSWNSGALTSGVHT |
| | | FPAVLQSSGLYSLKSVVTVPSSSLGTQTYICNVNHKPSNTKVDKR |
| | | VEPKSCDGGGGSGGGGSEVQLVESGGALVKPGGSLRLSCAASGF |
| | | TFTEAWMHWVRQAPGKQLEWVAQIKDRSQTYATYYAESVKGR |
| | | FTISRDDSKNTLYLQMNSLKTEDTAVYYCRGVYYANAPFDYWG |
| | | QGTLVTVSSASTKGPSVCPLAPSSKSTSGGTADLGCLVKDYFPEP |
| | | VTVSWNSGALTSGVHTFPAVLQSSGLYSLSSVVTVPSSSLGTQTY |
| | | ICNVNHKPSNTKVDKRVEPKSVDKTHTCPPCPAPPVAGPSVFLFP |
| | | PKPKDTLMISRTPEVTCVVVDVKHEDPEVKFNWYVDGVEVHNA |
| | | KTKPREEQYNSTYRVVSVLTVLHQDWLNGKEYKCKVSNKALPA |
| | | PIEKTISKAKGQPREPQVYTLPPCREEMTKNQVSLWCLVKGFYPS |
| | | DIAVEWESNGQPENNYKTTPPVLDSDGSFFLYSKLTVDKSRWQQ |
| | | GNVFSCSVMHEALHNHYTQKSLSLSPGK |
| 152 | TED4 LO12 | EIVMTQSPATLSVSPGERATLSCRASQSVASNLAWYQQKPGQAP |
| | Kappa Chain | RLLIYGASTRATGIPARFSGSGSGTEFTLTISSLQSEDFAVYYCQQ |
| | | YNNWPFTFGPGTKVDIKRTVAAPSVFIFPPSDEQLKSGTASVECLL |
| | | NNFYPREAKVQWKVDNALQSGNSQESVTEQDSKDSTYSLSSTLT |
| | | LSKADYEKHKVYACEVTHQGLSSPVTKSFNRGEC |
| 153 | TED4 LO12 | EVQLLESGGGLVQPGGSLRLSCAASGFTFDDYAIGWFRQAPGKE |
| | “hole” Heavy | REGVSCIRVSDGSTYYADSVKGRFTISRDNSKNTLYLQMNSLRAE |
| | Chain | DTAVYYCAAGSLYTCVQSIVWPARPYYDMDYWGQGTLVTVSSG |
| | | GGGSGGGGSQVQLQQSGPGLVKPSQTLSLTCAISGDSVSRNSAV |
| | | WNWIRQSPSRGLEWLGRTYYRSKWYNDYAVSVKSRITINPDTSK |
| | | NQFSLQLNSVTPEDTAVYYCARGLRQNQFYYYMDVWGKGTTVT |
| | | VSSASTKGPSVFPLAPSSKSTSGGTAALGCLVKDYFPEPVTVSWN |
| | | SGALTSGVHTFPAVLQSSGLYSLKSVVTVPSSSLGTQTYICNVNH |
| | | KPSNTKVDKRVEPKSCDKTHTCPPCPAPPVAGPSVFLFPPKPKDT |
| | | LMISRTPEVTCVVVDVKHEDPEVKFNWYVDGVEVHNAKTKPRE |
| | | EQYNSTYRVVSVLTVLHQDWLNGKEYKCKVSNKALPAPIEKTIS |
| | | KAKGQPREPQVCTLPPSREEMTKNQVSLSCAVKGFYPSDIAVEW |
| | | ESNGQPENNYKTTPPVLDSDGSFFLVSKLTVDKSRWQQGNVFSC |
| | | SVMHEALHNHYTQKSLSLSPGK |
| 154 | TED4 LO12 | DIVMTQSPLSLPVTPGEPASISCRSSQSLVHNTGNTYLSWYLQKPG |
| | Lambda | QSPQSLIYKVSNRASGVPDRFSGSGSGTDFTLKISRVEAEDVGVY |
| | Chain | YCGQGTQAPFTFGSGTKVEIKGQPKAAPSVRLFPPCSEELQANKA |
| | | TLVCLISDFYPGAVTVAWKADSSPVKAGVETTTPSKQSNNKYAA |
| | | SSYLSLTPEQWKSHRSYSCQVTHEGSTVEKTVAPTEVS |
| 155 | IgG1 Hinge | DKTHTCPPCPAPE |

1. A T cell engaging molecule comprising:
(a) an antigen binding arm that binds an epitope on human six transmembrane epithelial antigen of prostate-2 (STEAP2) and comprises a heavy chain comprising a heavy chain variable domain (VH) comprising a variable heavy chain complementarity determining region 1 (VH-CDR1) selected from SEQ ID NOs: 1, 9, 17, 103, 111, 127, 135, and 143; a VH-CDR2 selected from SEQ ID NOs: 2, 10, 18, 104, 112, 128, 136, and 144; a VH-CDR3 selected from SEQ ID NOs: 3, 11, 19, 94, 96, 98, 105, 113, 129, 137, and 145; a heavy chain CH1 domain; and a light chain comprising a light chain variable domain (VL) comprising a variable light chain complementarity determining region 1 (VL-CDR1) selected from SEQ ID NOs: 4, 12, 20, 100, 108, 130, 138, and 146; a VL-CDR2 selected from SEQ ID NOs: 5, 13, 21, 101, 109, 131, 139, and 147; a VL-CDR3 selected from SEQ ID NOs: 6, 14, 22, 102, 110, 132, 140, and 148; and a light chain constant domain;
(b) a first T cell binding arm that binds to cluster of differentiation 3 (CD3) comprising a heavy chain comprising a heavy chain variable domain (VH) comprising a VH-CDR1 selected from SEQ ID NOs: 36, 40, and 44; a VH-CDR2 selected from SEQ ID NOs: 37, 41, and 45; a VH-CDR3 selected from SEQ ID NOs: 38, 42, and 46; and a heavy chain CH1 domain; and a light chain comprising a light chain variable domain (VL) comprising a VL-CDR1 selected from SEQ ID NOs: 27 and 31; a VL-CDR2 selected from SEQ ID NOs: 28 and 32; a VL-CDR3 selected from SEQ ID NOs: 29 and 33; and a light chain constant domain; and
(c) a Fc domain, comprising a first Fc region and a second Fc region, each Fc region comprising a CH2 domain and a CH3 domain; said Fc domain further comprising at least one modification to promote heterodimerization.

2. A trivalent T cell engaging molecule comprising the T cell engaging molecule according to claim 1, further comprising (d) a second T cell binding arm that binds to cluster of differentiation 8 (CD8) and comprises a heavy chain comprising a heavy chain variable domain (VH) comprising a VH-CDR1 set forth in SEQ ID NO: 48, a VH-CDR2 set forth in SEQ ID NO: 49, and a VH-CDR3 set forth in SEQ ID NO: 50, and a heavy chain CH1 domain; and a light chain comprising a light chain variable domain (VL) comprising a VL-CDR1 set forth in SEQ ID NO: 51, a VL-CDR2 set forth in SEQ ID NO: 52, and a VL-CDR3 set forth in SEQ ID NO: 53, and a light chain constant domain.

3. The trivalent T cell engaging molecule according to claim 2, wherein the heavy chain of the second T cell binding arm is attached to the heavy chain of the first T cell binding arm through a linker.

4. The trivalent T cell engaging molecule according to claim 2, wherein the heavy chain of the second T cell binding arm is attached to the heavy chain of the antigen binding arm through a linker.

5. The trivalent T cell engaging molecule according to claim 3, wherein the linker comprises the amino acid sequence of SEQ ID NO: 89.

6. The trivalent T cell engaging molecule according to claim 5, wherein the linker comprises from 1 to about 10 copies of SEQ ID NO: 89.

7. The trivalent T cell engaging molecule according to claim 6, wherein the linker comprises 2 copies of SEQ ID NO: 89.

8. The T cell engaging molecule or trivalent T cell engaging molecule according to claim 1, wherein one of the two CH3 domains comprises a knob mutation, and the other of the two CH3 domains comprises a hole mutation.

9. The T cell engaging molecule or trivalent T cell engaging molecule according to claim 1, wherein the CH1 domain and the light chain constant domain of one or more of each of the (a) antigen binding arm, (b) the first T cell binding arm, and (d) the second T cell binding arm further comprises a charge pair substitution comprising a first charged amino acid substitution in the CH1 domain and a second charged amino acid substitution in the light chain constant domain, wherein the first charged amino acid substitution and the second charged amino acid substitution have an opposite charge.

10. The T cell engaging molecule or trivalent T cell engaging molecule according to claim 9, wherein the light chain constant domain of one or more of each of (a), (b), and (d) is a lambda light chain constant domain (CLλ) and wherein the charge pair is a lambda charge pair comprising a positively charged amino acid residue selected from arginine, lysine or histidine, and a negatively charged amino acid residue selected from aspartic acid, glutamic acid, serine or threonine, wherein the numbering is according to the EU index and wherein the charged amino acids of the lambda charge pair are located at one or more of the following positions:
(i) position 117 in the CLλ and position 141 in the CH1 domain;
(ii) position 117 in the CLλ and position 185 in the CH1 domain;
(iii) position 119 in the CLλ and position 128 in the CH1 domain;
(iv) position 134 in the CLλ and position 128 in the CH1 domain;
(v) position 134 in the CLλ and position 145 in the CH1 domain;
(vi) position 134 in the CLλ and position 183 in the CH1 domain;
(vii) position 136 in the CLλ and position 185 in the CH1 domain;
(viii) position 178 in the CLλ and position 173 in the CH1 domain; and/or
(ix) position 117 in the CLλ and position 187 in the CH1 domain.

11. The T cell engaging molecule or trivalent T cell engaging molecule according to claim 10, wherein (i) the charged amino acid at position 117 is arginine and the charged amino acid at position 141 is aspartic acid; the charged amino acid at position 117 is arginine and the charged amino acid at position 141 is glutamic acid; the charged amino acid at position 117 is arginine and the charged amino acid at position 141 is serine; the charged amino acid at position 117 is arginine and the charged amino acid at position 141 is threonine; the charged amino acid at position 117 is lysine and the charged amino acid at position 141 is aspartic acid; the charged amino acid at position 117 is lysine and the charged amino acid at position 141 is glutamic acid; the charged amino acid at position 117 is lysine and the charged amino acid at position 141 is serine; or the charged amino acid at position 117 is lysine and the charged amino acid at position 141 is threonine;
(ii) the charged amino acid at position 117 is arginine and the charged amino acid at position 185 is aspartic acid; the charged amino acid at position 117 is arginine and the charged amino acid at position 185 is glutamic acid; the charged amino acid at position 117 is arginine and the charged amino acid at position 185 is serine; the charged amino acid at position 117 is arginine and the charged amino acid at position 185 is threonine; the charged amino acid at position 117 is lysine and the charged amino acid at position 185 is aspartic acid; the charged amino acid at position 117 is lysine and the charged amino acid at position 185 is glutamic acid; the charged amino acid at position 117 is lysine and the charged amino acid at position 185 is serine; or the charged amino acid at position 117 is lysine and the charged amino acid at position 185 is threonine;
(iii) the charged amino acid at position 119 is arginine and the charged amino acid at position 128 is aspartic acid; the charged amino acid at position 119 is arginine and the charged amino acid at position 128 is glutamic acid; the charged amino acid at position 119 is arginine and the charged amino acid at position 128 is serine; the charged amino acid at position 119 is arginine and the charged amino acid at position 128 is threonine; the charged amino acid at position 119 is lysine and the charged amino acid at position 128 is aspartic acid; the charged amino acid at position 119 is lysine and the charged amino acid at position 128 is glutamic acid; the charged amino acid at position 119 is lysine and the charged amino acid at position 128 is serine; or the charged amino acid at position 119 is lysine and the charged amino acid at position 128 is threonine;
(iv) the charged amino acid at position 134 is arginine and the charged amino acid at position 128 is aspartic acid; the charged amino acid at position 134 is arginine and the charged amino acid at position 128 is glutamic acid; the charged amino acid at position 134 is arginine and the charged amino acid at position 128 is serine; the charged amino acid at position 134 is arginine and the charged amino acid at position 128 is threonine; the charged amino acid at position 134 is lysine and the charged amino acid at position 128 is aspartic acid; the charged amino acid at position 134 is lysine and the charged amino acid at position 128 is glutamic acid; the charged amino acid at position 134 is lysine and the charged amino acid at position 128 is serine; or the charged amino acid at position 134 is lysine and the charged amino acid at position 128 is threonine;
(v) the charged amino acid at position 134 is arginine and the charged amino acid at position 145 is aspartic acid; the charged amino acid at position 134 is arginine and the charged amino acid at position 145 is glutamic acid; the charged amino acid at position 134 is arginine and the charged amino acid at position 145 is serine; the charged amino acid at position 134 is arginine and the charged amino acid at position 145 is threonine; the charged amino acid at position 134 is lysine and the charged amino acid at position 145 is aspartic acid; the charged amino acid at position 134 is lysine and the charged amino acid at position 145 is glutamic acid; the charged amino acid at position 134 is lysine and the charged amino acid at position 145 is serine; or the charged amino acid at position 134 is lysine and the charged amino acid at position 145 is threonine;
(vi) the charged amino acid at position 134 is arginine and the charged amino acid at position 183 is aspartic acid; the charged amino acid at position 134 is arginine and the charged amino acid at position 183 is glutamic acid; the charged amino acid at position 134 is arginine and the charged amino acid at position 183 is serine; the charged amino acid at position 134 is arginine and the charged amino acid at position 183 is threonine; the charged amino acid at position 134 is lysine and the charged amino acid at position 183 is aspartic acid; the charged amino acid at position 134 is lysine and the charged amino acid at position 183 is glutamic acid; the charged amino acid at position 134 is lysine and the charged amino acid at position 183 is serine; or the charged amino acid at position 134 is lysine and the charged amino acid at position 183 is threonine;
(vii) the charged amino acid at position 136 is arginine and the charged amino acid at position 185 is aspartic acid; the charged amino acid at position 136 is arginine and the charged amino acid at position 185 is glutamic acid; the charged amino acid at position 136 is arginine and the charged amino acid at position 185 is serine; the charged amino acid at position 136 is arginine and the charged amino acid at position 185 is threonine; the charged amino acid at position 136 is lysine and the charged amino acid at position 185 is aspartic acid; the charged amino acid at position 136 is lysine and the charged amino acid at position 185 is glutamic acid; the charged amino acid at position 136 is lysine and the charged amino acid at position 185 is serine; or the charged amino acid at position 136 is lysine and the charged amino acid at position 185 is threonine;
(viii) the charged amino acid at position 178 is arginine and the charged amino acid at position 173 is aspartic acid; the charged amino acid at position 178 is arginine and the charged amino acid at position 173 is glutamic acid; the charged amino acid at position 178 is arginine and the charged amino acid at position 173 is serine; the charged amino acid at position 178 is arginine and the charged amino acid at position 173 is threonine; the charged amino acid at position 178 is lysine and the charged amino acid at position 173 is aspartic acid; the charged amino acid at position 178 is lysine and the charged amino acid at position 173 is glutamic acid; the charged amino acid at position 178 is lysine and the charged amino acid at position 173 is serine; or the charged amino acid at position 178 is lysine and the charged amino acid at position 173 is threonine; and/or
(ix) the charged amino acid at position 117 is arginine and the charged amino acid at position 187 is aspartic acid; the charged amino acid at position 117 is arginine and the charged amino acid at position 187 is glutamic acid; the charged amino acid at position 117 is arginine and the charged amino acid at position 187 is serine; the charged amino acid at position 117 is arginine and the charged amino acid at position 187 is threonine; the charged amino acid at position 117 is lysine and the charged amino acid at position 187 is aspartic acid; the charged amino acid at position 117 is lysine and the charged amino acid at position 187 is glutamic acid; the charged amino acid at position 117 is lysine and the charged amino acid at position 187 is serine; or the charged amino acid at position 117 is lysine and the charged amino acid at position 187 is threonine.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The trivalent T cell engaging molecule according to claim 1, wherein the trivalent T cell engaging molecule comprises SEQ ID NOs: 25, 26, 35, 55 and 58.

38. A trivalent T cell engaging molecule comprising:
(a) a first antigen binding arm and a second antigen binding arm that each bind an epitope on human six transmembrane epithelial antigen of prostate-2 (STEAP2) and each comprise a heavy chain comprising a heavy chain variable domain (VH) comprising a variable heavy chain complementarity determining region 1 (VH-CDR1) selected from SEQ ID NOs: 1, 9, 17, 103, 111, 127, 135, and 143; a VH-CDR2 selected from SEQ ID NOs: 2, 10, 18, 104, 112, 128, 136, and 144; a VH-CDR3 selected from SEQ ID NOs: 3, 11, 19, 94, 96, 98, 105, 113, 129, 137, and 145; a heavy chain CH1 domain; and a light chain comprising a light chain variable domain (VL) comprising a variable light chain complementarity determining region 1 (VL-CDR1) selected from SEQ ID NOs: 4, 12, 20, 100, 108, 130, 138, and 146; a VL-CDR2 selected from SEQ ID NOs: 5, 13, 21, 101, 109, 131, 139, and 147; a VL-CDR3 selected from SEQ ID NOs: 6, 14, 22, 102, 110, 132, 140, and 148; and a light chain constant domain;
(b) a first T cell binding arm that binds to cluster of differentiation 3 (CD3) and comprises a heavy chain comprising a VH comprising a VH-CDR1 selected from SEQ ID NOs: 36, 40, and 44; a VH-CDR2 selected from SEQ ID NOs: 37, 41, and 45; a VH-CDR3 selected from SEQ ID NOs: 38, 42, and 46; and a heavy chain CH1 domain; and a light chain comprising a VL comprising a VL-CDR1 selected from SEQ ID NOs: 27 and 31; a VL-CDR2 selected from SEQ ID NOs: 28 and 32; a VL-CDR3 selected from SEQ ID NOs: 29 and 33; and a light chain constant domain; and
(c) an Fc domain, comprising a first Fc region and a second Fc region, each Fc region comprising a CH2 domain and a CH3 domain; said Fc domain further comprising at least one modification to promote heterodimerization;
wherein the heavy chain of the first antigen binding arm and the heavy chain of the first T cell binding arm are attached to the Fc domain, and the heavy chain of the second antigen binding arm is attached to the heavy chain of the first T cell binding arm.

39. A tetravalent T cell engaging molecule comprising the trivalent T cell engaging molecule according to claim 38, further comprising (d) a second T cell binding arm that binds to cluster of differentiation 8 (CD8) and comprises a heavy chain comprising a variable heavy domain (VHH) comprising a VH-CDR1 set forth in SEQ ID NO: 84; a VH-CDR2 set forth in SEQ ID NO: 85; and a VH-CDR3 set forth in SEQ ID NO: 86.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. The tetravalent T cell engaging molecule according to claim 39, wherein the tetravalent T cell engaging molecule comprises SEQ ID NOs: 25, 35, 81, 25, and 87, or wherein the tetravalent T cell engaging molecule comprises SEQ ID NOs: 151, 152, 153, 154, and 152.

77. One or more nucleic acid(s) encoding the T cell engaging molecule of claim 76.

78. A vector comprising the nucleic acid(s) of claim 77.

79. An isolated host cell comprising the nucleic acid(s) of claim 77.

80. A pharmaceutical composition comprising the T cell engaging molecule according to claim 76 and a pharmaceutically acceptable carrier.

81. A method of treating a disease in a patient in need thereof, the method comprising administering to the patient an effective amount of the T cell engaging molecule according to claim 76.

82. (canceled)

83. (canceled)

84. (canceled)

85. (canceled)


SEQUENCE LISTING (155 sequences)

SEQ ID NO: 1 - protein, 5 aa, synthetic construct
  source 1..5: note = STEAP2 30D12 G96P VH CDR1
SYGMS

SEQ ID NO: 2 - protein, 17 aa, synthetic construct
  source 1..17: note = STEAP2 30D12 G96P VH CDR2
TISSGGSYTFYPDIMKG

SEQ ID NO: 3 - protein, 13 aa, synthetic construct
  source 1..13: note = STEAP2 30D12 G96P VH CDR3
RPYGTIYTFSFDA

SEQ ID NO: 4 - protein, 16 aa, synthetic construct
  source 1..16: note = STEAP2 30D12 G96P VL CDR1
RSSQSVVHSNANTYLE

SEQ ID NO: 5 - protein, 7 aa, synthetic construct
  source 1..7: note = STEAP2 30D12 G96P VL CDR2
KVSNRFS

SEQ ID NO: 6 - protein, 9 aa, synthetic construct
  source 1..9: note = STEAP2 30D12 G96P VL CDR3
FQGSHVPYT

SEQ ID NO: 7 - protein, 122 aa, synthetic construct
  source 1..122: note = STEAP2 30D12 G96P VH
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRPYGTIYTFSFDAWGQGTTLTVSS

SEQ ID NO: 8 - protein, 112 aa, synthetic construct
  source 1..112: note = STEAP2 30D12 G96P VL
DVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIK

SEQ ID NO: 9 - protein, 5 aa, synthetic construct
  source 1..5: note = STEAP2-2 VH CDR1
SYGMS

SEQ ID NO: 10 - protein, 17 aa, synthetic construct
  source 1..17: note = STEAP2-2 VH CDR2
TISSGGSYTFYPDIMKG

SEQ ID NO: 11 - protein, 13 aa, synthetic construct
  source 1..13: note = STEAP2-2 VH CDR3
RPYGTIYTFSFDS

SEQ ID NO: 12 - protein, 16 aa, synthetic construct
  source 1..16: note = STEAP2-2 VL CDR1
RSSQSVVHSNGNTYLE

SEQ ID NO: 13 - protein, 7 aa, synthetic construct
  source 1..7: note = STEAP2-2 VL CDR2
KVSNRFS

SEQ ID NO: 14 - protein, 9 aa, synthetic construct
  source 1..9: note = STEAP2-2 VL CDR3
FQGSHVPYT

SEQ ID NO: 15 - protein, 122 aa, synthetic construct
  source 1..122: note = STEAP2-2 G96P VH
EVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRPYGTIYTFSFDSWGQGTTLTVSS

SEQ ID NO: 16 - protein, 112 aa, synthetic construct
  source 1..112: note = STEAP2-2 VL
DVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 17 - protein, 5 aa, synthetic construct
  source 1..5: note = STEAP2-2 Humanized2 VH CDR1
SYGMS

SEQ ID NO: 18 - protein, 17 aa, synthetic construct
  source 1..17: note = STEAP2-2 Humanized2 VH CDR2
TISSGGSYTFYPDIMKG

SEQ ID NO: 19 - protein, 13 aa, synthetic construct
  source 1..13: note = STEAP2-2 Humanized2 G96P VH CDR3
RPYGTIYTFSFDA

SEQ ID NO: 20 - protein, 16 aa, synthetic construct
  source 1..16: note = STEAP2-2 Humanized2 VL CDR1
RSSQSVVHSNANTYLE

SEQ ID NO: 21 - protein, 7 aa, synthetic construct
  source 1..7: note = STEAP2-2 Humanized2 VL CDR2
KVSNRFS

SEQ ID NO: 22 - protein, 9 aa, synthetic construct
  source 1..9: note = STEAP2-2 Humanized2 VL CDR3
FQGSHVPYT

SEQ ID NO: 23 - protein, 122 aa, synthetic construct
  source 1..122: note = STEAP2-2 G96P VH
EVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRPYGTIYTFSFDAWGQGTTLTVSS

SEQ ID NO: 24 - protein, 112 aa, synthetic construct; identical to SEQ ID NO: 8
  source 1..112: note = STEAP2-2 Humanized2 VL

SEQ ID NO: 25 - protein, 219 aa, synthetic construct
  source 1..219: note = 30D12-TED3 Light Chain
DVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 452 aa, synthetic construct
  source 1..452: note = 30D12-TED/TED3 hole heavy chain
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRPYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 16 aa, synthetic construct
  source 1..16: note = CD3 Vk17 CDR1
RSSQSLVHNTGNTYLS

SEQ ID NO: 28 - protein, 7 aa, synthetic construct
  source 1..7: note = CD3 Vk17 CDR2
KVSNRAS

SEQ ID NO: 29 - protein, 9 aa, synthetic construct
  source 1..9: note = CD3 Vk17 CDR3
GQGTQAPFT

SEQ ID NO: 30 - protein, 112 aa, synthetic construct
  source 1..112: note = Vk17
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQAPFTFGSGTKVEIK

SEQ ID NO: 31 - protein, 16 aa, synthetic construct
  source 1..16: note = CD3 Vk29 CDR1
RSSQSLVHNTGNTYLS

SEQ ID NO: 32 - protein, 7 aa, synthetic construct
  source 1..7: note = CD3 Vk29 CDR2
KVSNRAS

SEQ ID NO: 33 - protein, 9 aa, synthetic construct
  source 1..9: note = CD3 Vk29 CDR3
GQGTQFPFT

SEQ ID NO: 34 - protein, 112 aa, synthetic construct
  source 1..112: note = Vk29
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQFPFTFGSGTKVEIK

SEQ ID NO: 35 - protein, 218 aa, synthetic construct
  source 1..218: note = TED3 CD3 Light Chain K29
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQFPFTFGSGTKVEIKGQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 36 - protein, 5 aa, synthetic construct
  source 1..5: note = CD3 VH CDR1 SN75
NAWMH

SEQ ID NO: 37 - protein, 20 aa, synthetic construct
  source 1..20: note = CD3 VH CDR2 SN75
QIKDRSQTYATYYAESVKGR

SEQ ID NO: 38 - protein, 12 aa, synthetic construct
  source 1..12: note = CD3 VH CDR3 SN75
RGVYYANAPFDY

SEQ ID NO: 39 - protein, 121 aa, synthetic construct
  source 1..121: note = CD3 VH SN75
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYANAPFDYWGQGTLVTVSS

SEQ ID NO: 40 - protein, 5 aa, synthetic construct
  source 1..5: note = CD3 VH CDR1 SN79
NAWMH

SEQ ID NO: 41 - protein, 20 aa, synthetic construct
  source 1..20: note = CD3 VH CDR2 SN79
QIKDRSQTYATYYAESVKGR

SEQ ID NO: 42 - protein, 12 aa, synthetic construct
  source 1..12: note = CD3 VH CDR3 SN79
RGVYYAVTPFDY

SEQ ID NO: 43 - protein, 121 aa, synthetic construct
  source 1..121: note = CD3 VH SN79
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYAVTPFDYWGQGTLVTVSS

SEQ ID NO: 44 - protein, 5 aa, synthetic construct
  source 1..5: note = CD3 VH CDR1 E75
EAWMH

SEQ ID NO: 45 - protein, 20 aa, synthetic construct
  source 1..20: note = CD3 VH CDR2 E75
QIKDRSQTYATYYAESVKGR

SEQ ID NO: 46 - protein, 12 aa, synthetic construct
  source 1..12: note = CD3 VH CDR3 E75
RGVYYANAPFDY

SEQ ID NO: 47 - protein, 121 aa, synthetic construct
  source 1..121: note = CD3 VH E75
EVQLVESGGALVKPGGSLRLSCAASGFTFTEAWMHWVRQAPGKQLEWVAQIKDRSQTYATYYAESVKGRFTISRDDSKNTLYLQMNSLKTEDTAVYYCRGVYYANAPFDYWGQGTLVTVSS

SEQ ID NO: 48 - protein, 5 aa, synthetic construct
  source 1..5: note = CD8 VH CDR1
DFGMN

SEQ ID NO: 49 - protein, 17 aa, synthetic construct
  source 1..17: note = CD8 VH CDR2
LIYYDGSNKFYADSVKG

SEQ ID NO: 50 - protein, 12 aa, synthetic construct
  source 1..12: note = CD8 VH CDR3
PHYDGYYHFFDS

SEQ ID NO: 51 - protein, 11 aa, synthetic construct
  source 1..11: note = CD8 VL CDR1
KGSQDINNYLA

SEQ ID NO: 52 - protein, 7 aa, synthetic construct
  source 1..7: note = CD8 VL CDR2
NTDILHT

SEQ ID NO: 53 - protein, 8 aa, synthetic construct
  source 1..8: note = CD8 VL CDR3
YQYNNGYT

SEQ ID NO: 54 - protein, 106 aa, synthetic construct
  source 1..106: note = CD8 VL
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 55 - protein, 213 aa, synthetic construct
  source 1..213: note = CD8 LC VL SEQ ID NO: 54 + mutant Ck SEQ ID NO: 64
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPCDEQLKSGTASVKCLLNNFYPREAKVQWKVDNALQSGNSQESVTEQDSKDSTYSLSSTLTLSKADYEKHKVYACEVTHQGLSSPVTKSFNRGEV

SEQ ID NO: 56 - protein, 121 aa, synthetic construct
  source 1..121: note = CD8 VH
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 57 - protein, 104 aa, synthetic construct
  source 1..104: note = CD8 CH1 CH1-portion of SEQ ID NO: 78 w/ S183E
ASTKGPSVCPLAPSSKSTSGGTAALGCLVKDYFPEPVTVSWNSGALTSGVHTFPAVLQSSGLYSLESVVTVPSSSLGTQTYICNVNHKPSNTKVDKRVEPKSVD

SEQ ID NO: 58 - protein, 686 aa, synthetic construct
  source 1..686: note = knob HC SEQ ID NO: 56 CD8 VH + SEQ ID NO: 57 CD8 CH1 + SEQ ID NO: 89 linker + SEQ ID NO: 39 CD3 VH SN75 + SEQ ID NO: 79 IgG1 HC
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVCPLAPSSKSTSGGTAALGCLVKDYFPEPVTVSWNSGALTSGVHTFPAVLQSSGLYSLESVVTVPSSSLGTQTYICNVNHKPSNTKVDKRVEPKSVDGGGGSGGGGSEVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYANAPFDYWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 106 aa, synthetic construct
  source 1..106: note = wt C[lambda]
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 60 - protein, 106 aa, synthetic construct
  source 1..106: note = V12 C[lambda] (S122C, C212V)
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 61 - protein, 106 aa, synthetic construct
  source 1..106: note = V12C[lambda] of SEQ ID NO: 60 w/ T117R
GQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 62 - protein, 107 aa, synthetic construct [sequence omitted]

SEQ ID NO: 63 - protein, 107 aa, synthetic construct
  source 1..107: note = wt Ck of SEQ ID NO: 62 w/ V133E
RTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 107 aa, synthetic construct
  source 1..107: note = mutant Ck S121C, V133K, C214V
RTVAAPSVFIFPPCDEQLKSGTASVKCLLNNFYPREAKVQWKVDNALQSGNSQESVTEQDSKDSTYSLSSTLTLSKADYEKHKVYACEVTHQGLSSPVTKSFNRGEV

SEQ ID NO: 65 - protein, 103 aa, synthetic construct
  source 1..103: note = IgG1 wt CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 66 - protein, 103 aa, synthetic construct
  source 1..103: note = V12 CH1 (F126C, C220V)
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSV

SEQ ID NO: 67 - protein, 103 aa, synthetic construct
  source 1..103: note = wt CH1 of SEQ ID NO: 65 w/ S183K
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSC

SEQ ID NO: 68 - protein, 104 aa, synthetic construct
  source 1..104: note = IgG1 wt CH2
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 69 - protein, 104 aa, synthetic construct
  source 1..104: note = wt CH2 of SEQ ID NO: 68 w/ L234F, L235E, and P331S
FEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTIS

SEQ ID NO: 70 - protein, 107 aa, synthetic construct
  source 1..107: note = IgG1wt CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 330 aa, synthetic construct
  source 1..330: note = IgG1 wt constant domain HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 107 aa, synthetic construct
  source 1..107: note = IgG1 CH3 T366S, L368A, Y407V [hole]
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 107 aa, synthetic construct
  source 1..107: note = IgG1 CH3 Y349C T366S, L368A, Y407V
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 107 aa, synthetic construct
  source 1..107: note = IgG1 CH3 T366W [knob]
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 107 aa, synthetic construct
  source 1..107: note = IgG1 CH3 S354C, T366W
GQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 330 aa, synthetic construct
  source 1..330: note = IgG1 hole T366S, L368A, Y407V, Y349C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 77 - protein, 330 aa, synthetic construct
  source 1..330: note = IgG1 HC knob CH1, CH2, CH3 T366W, S354C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 330 aa, synthetic construct
  source 1..330: note = V12 with stabilizing cysteine, interchain cysteine mutations, and knob mutations : T366W, F126C, C220V, S354C
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 330 aa, synthetic construct
  source 1..330: note = SEQ ID NO: 78 w/ A141D, L234F, L235E, P331S
ASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 330 aa, synthetic construct
  source 1..330: note = IgG1 hole w/ CH1 F126C, C220V + CH2/CH3 of SEQ ID NO: 76
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 81 - protein, 686 aa, synthetic construct
  source 1..686: note = STEAP2/CD3 knob HC: SEQ ID NO: 7 VH STEAP2 + SEQ ID NO: 67 wt CH1 w/ S183K + D + SEQ ID NO: 86 linker + SEQ ID NO: 39 CD3 VH SN75 + SEQ ID NO: 82.
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRPYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDGGGGSGGGGSEVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYANAPFDYWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 329 aa, synthetic construct
  source 1..329: note = CH1 w/ A141D, CH2, being IgG2 at 233-236, S267K, and wt P at 331; CH3 S354C, T366W.
ASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 131 aa, synthetic construct
  source 1..131: note = CD8 VHH
EVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSS

SEQ ID NO: 84 - protein, 6 aa, synthetic construct
  source 1..6: note = CD8 VHH CDR1
DDYAIG

SEQ ID NO: 85 - protein, 17 aa, synthetic construct
  source 1..17: note = CD8 VHH CDR2
CIRVSDGSTYYADSVKG

SEQ ID NO: 86 - protein, 22 aa, synthetic construct
  source 1..22: note = CD8 VHH CDR3
GSLYTCVQSIVWPARPYYDMDY

SEQ ID NO: 87 - protein, 592 aa, synthetic construct
  source 1..592: note = hole CD8 VHH STEAP2 HC: SEQ ID NO: 83 CD8 VHH + SEQ ID NO: 89 linker + SEQ ID NO: 7 STEAP2 VH + SEQ ID NO: 88
EVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSGGGGSEVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRPYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 329 aa, synthetic construct
  source 1..329: note = IgG1 hole of SEQ ID NO: 76 w/ S183K, CH2 being IgG2 at 233-236, and S267K
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 89 - protein, 5 aa, synthetic construct
  source 1..5: note = linker
GGGGS

SEQ ID NO: 90 - protein, 225 aa, synthetic construct
  source 1..225: note = STEAP2 VH + CH1: SEQ ID NO: 7 + SEQ ID NO: 67
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRPYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSC

SEQ ID NO: 91 - protein, 330 aa, synthetic construct
  source 1..330: note = STEAP2 hole CH1 SEQ ID NO: 67, CH2 SEQ ID NO: 69, CH3 SEQ ID NO: 73
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 451 aa, synthetic construct
  source 1..451: note = knob HC for TED SEQ ID NO: 39 CD3 VH SN75 + SEQ ID NO: 79 IgG1 HC
EVQLVESGGALVKPGGSLRLSCAASGFTFSNAWMHWVRQAPGKQLEWVAQIKDRSQTYATYYAESVKGRFTISRDDSKNTLYLQMNSLKTEDTAVYYCRGVYYANAPFDYWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 451 aa, synthetic construct
  source 1..451: note = hole STEAP2 HC for TED2: SEQ ID NO: 7 STEAP2 VH + SEQ ID NO: 88
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRPYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 13 aa, synthetic construct
  source 1..13: note = STEAP2-2 Humanized1 VH CDR3
RGYGTIYTFSFDA

SEQ ID NO: 95 - protein, 122 aa, synthetic construct
  source 1..122: note = STEAP2-2 Humanized1 VH
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSS

SEQ ID NO: 96 - protein, 13 aa, synthetic construct
  source 1..13: note = STEAP2-2 VH CDR3
RGYGTIYTFSFDS

SEQ ID NO: 97 - protein, 122 aa, synthetic construct
  source 1..122: note = STEAP2-2 VH
EVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRGYGTIYTFSFDSWGQGTTLTVSS

SEQ ID NO: 98 - protein, 13 aa, synthetic construct
  source 1..13: note = STEAP2-2 Humanized2 VH CDR3
RGYGTIYTFSFDA

SEQ ID NO: 99 - protein, 122 aa, synthetic construct
  source 1..122: note = STEAP2-2 Humanized2 VH
EVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRGYGTIYTFSFDAWGQGTTLTVSS

SEQ ID NO: 100 - protein, 11 aa, synthetic construct
  source 1..11: note = STEAP2-8 VL CDR1
RASQSVASNLA

SEQ ID NO: 101 - protein, 7 aa, synthetic construct
  source 1..7: note = STEAP2-8 VL CDR2
GASTRAT

SEQ ID NO: 102 - protein, 9 aa, synthetic construct
  source 1..9: note = STEAP2-8 VL CDR3
QQYNNWPFT

SEQ ID NO: 103 - protein, 7 aa, synthetic construct
  source 1..7: note = STEAP2-8 VH CDR1
RNSAVWN

SEQ ID NO: 104 - protein, 18 aa, synthetic construct
  source 1..18: note = STEAP2-8 VH CDR2
RTYYRSKWYNDYAPSVKS

SEQ ID NO: 105 - protein, 13 aa, synthetic construct
  source 1..13: note = STEAP2-8 VH CDR3
GLRQNQFYYYMDV

SEQ ID NO: 106 - protein, 125 aa, synthetic construct
  source 1..125: note = STEAP2-8 VH
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSS

SEQ ID NO: 107 - protein, 107 aa, synthetic construct
  source 1..107: note = STEAP2-8 VL
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIK

SEQ ID NO: 108 - protein, 11 aa, synthetic construct
  source 1..11: note = STEAP2 40A3 VL CDR1
RASQSVNSNLA

SEQ ID NO: 109 - protein, 7 aa, synthetic construct
  source 1..7: note = STEAP2 40A3 VL CDR2
GASTRAT

SEQ ID NO: 110 - protein, 9 aa, synthetic construct
  source 1..9: note = STEAP2 40A3 VL CDR3
QQYNNWPFT

SEQ ID NO: 111 - protein, 7 aa, synthetic construct
  source 1..7: note = STEAP2 40A3 VH CDR1
RNSAVWN

SEQ ID NO: 112 - protein, 18 aa, synthetic construct
  source 1..18: note = STEAP2 40A3 VH CDR2
RTYYRSKWYNDYAVSVKS

SEQ ID NO: 113 - protein, 13 aa, synthetic construct
  source 1..13: note = STEAP2 40A3 VH CDR3
GLLQNNFYYYMDV

SEQ ID NO: 114 - protein, 125 aa, synthetic construct
  source 1..125: note = STEAP2 40A3 VH
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 115 - protein, 107 aa, synthetic construct
  source 1..107: note = STEAP2 40A3 VL
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIK

SEQ ID NO: 116 - protein, 219 aa, synthetic construct
  source 1..219: note = STEAP2 LC: SEQ ID NO: 16 + SEQ ID NO: 63 [wt Ck w/ V133E]
DVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 219 aa, synthetic construct; identical to SEQ ID NO: 25
  source 1..219: note = STEAP2 LC: SEQ ID NO: 24 + SEQ ID NO: 63 [wt Ck w/ V133E]

SEQ ID NO: 118 - protein, 214 aa, synthetic construct
  source 1..214: note = STEAP2 LC: SEQ ID NO: 107 + SEQ ID NO: 63 [wt Ck] w/ V133E]
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 214 aa, synthetic construct
  source 1..214: note = STEAP2 LC: SEQ ID NO: 115 + SEQ ID NO: 63 [wt Ck w/ V133E]
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKRTVAAPSVFIFPPSDEQLKSGTASVECLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 452 aa, synthetic construct
  source 1..452: note = STEAP2 HC hole: STEAP2 VH SEQ ID NO: 15 + SEQ ID NO: 67 wt CH1 w/ S183K + SEQ ID NO: 69 + SEQ ID NO: 73
EVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRPYGTIYTFSFDSWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 121 - protein, 452 aa, synthetic construct
  source 1..452: note = STEAP2 HC hole: STEAP2 VH SEQ ID NO: 23 + SEQ ID NO: 67 wt CH1 w/ S183K + SEQ ID NO: 69 + SEQ ID NO: 73
EVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRPYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 122 - protein, 452 aa, synthetic construct
  source 1..452: note = STEAP2 HC hole: STEAP2 VH SEQ ID NO: 95 + SEQ ID NO: 67 wt CH1 w/ S183K + SEQ ID NO: 69 + SEQ ID NO: 73
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 123 - protein, 452 aa, synthetic construct
  source 1..452: note = STEAP2 HC hole: STEAP2 VH SEQ ID NO: 97 + SEQ ID NO: 67 + SEQ ID NO: 69 + SEQ ID NO: 73
EVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRGYGTIYTFSFDSWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 124 - protein, 452 aa, synthetic construct
  source 1..452: note = STEAP2 HC hole: STEAP2 VH SEQ ID NO: 99 + SEQ ID NO: 67 + SEQ ID NO: 69 + SEQ ID NO: 73
EVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRGYGTIYTFSFDAWGQGTTLTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 125 - protein, 455 aa, synthetic construct
  source 1..455: note = STEAP2 HC hole: STEAP2 VH SEQ ID NO: 106 + SEQ ID NO: 67 + SEQ ID NO: 69 + SEQ ID NO: 73
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 126 - protein, 455 aa, synthetic construct
  source 1..455: note = STEAP2 HC hole: STEAP2 VH SEQ ID NO: 114 + SEQ ID NO: 67 + SEQ ID NO: 69 + SEQ ID NO: 73
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 127 - protein, 7 aa, synthetic construct
  source 1..7: note = STEAP2-3 VH CDR1
RNSAVWN

SEQ ID NO: 128 - protein, 18 aa, synthetic construct
  source 1..18: note = STEAP2-3 VH CDR2
RTYYRSKWYNDYAVSVKS

SEQ ID NO: 129 - protein, 13 aa, synthetic construct
  source 1..13: note = STEAP2-3 VH CDR3
GLLQNQFYYYMDV

SEQ ID NO: 130 - protein, 11 aa, synthetic construct
  source 1..11: note = STEAP2-3 VL CDR1(40A3GL-LO7)
RASQSVSSNLA

SEQ ID NO: 131 - protein, 7 aa, synthetic construct
  source 1..7: note = STEAP2-3 VL CDR2
GASTRAT

SEQ ID NO: 132 - protein, 9 aa, synthetic construct
  source 1..9: note = STEAP2-3 VL CDR3
QQYNNWPFT

SEQ ID NO: 133 - protein, 125 aa, synthetic construct
  source 1..125: note = STEAP2-3 VH
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSS

SEQ ID NO: 134 - protein, 107 aa, synthetic construct
  source 1..107: note = STEAP2-3 VL
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 135 - protein, 7 aa, synthetic construct
  source 1..7: note = 40A3GL-LO11 VH CDR 1
RNSAVWN

SEQ ID NO: 136 - protein, 18 aa, synthetic construct
  source 1..18: note = 40A3GL-LO11 VH CDR 2
RTYYRSKWYNDYAPSVKS

SEQ ID NO: 137 - protein, 13 aa, synthetic construct
  source 1..13: note = 40A3GL-LO11 VH CDR 3
GLLQNQFYYYMDV

SEQ ID NO: 138 - protein, 11 aa, synthetic construct
  source 1..11: note = 40A3GL-LO11 VL CDR 1
RASQSVASNLA

SEQ ID NO: 139 - protein, 7 aa, synthetic construct
  source 1..7: note = 40A3GL-LO11 VL CDR 2
GASTRAT

SEQ ID NO: 140 - protein, 9 aa, synthetic construct
  source 1..9: note = 40A3GL-LO11 VL CDR 3
QQYNNWPFT

SEQ ID NO: 141 - protein, 125 aa, synthetic construct
  source 1..125: note = 40A3GL-LO11 VH
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLLQNQFYYYMDVWGKGTTVTVSS

SEQ ID NO: 142 - protein, 107 aa, synthetic construct; identical to SEQ ID NO: 107
  source 1..107: note = 40A3GL-LO11 VL

SEQ ID NO: 143 - protein, 7 aa, synthetic construct
  source 1..7: note = STEAP2 40A3 LO12 VH CDR1
RNSAVWN

SEQ ID NO: 144 - protein, 18 aa, synthetic construct
  source 1..18: note = STEAP2 40A3 LO12 VH CDR2
RTYYRSKWYNDYAVSVKS

SEQ ID NO: 145 - protein, 13 aa, synthetic construct
  source 1..13: note = STEAP2 40A3 LO12 VH CDR3
GLRQNQFYYYMDV

SEQ ID NO: 146 - protein, 11 aa, synthetic construct
  source 1..11: note = STEAP240A3 LO12 VL CDR1
RASQSVASNLA

SEQ ID NO: 147 - protein, 7 aa, synthetic construct
  source 1..7: note = STEAP2a40A3 LO12 VL CDR2
GASTRAT

SEQ ID NO: 148 - protein, 9 aa, synthetic construct
  source 1..9: note = STEAP2a 40A3 LO12 VL CDR3
QQYNNWPFT

SEQ ID NO: 149 - protein, 125 aa, synthetic construct
  source 1..125: note = STEAP2 VH 40A3 LO12
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSS

SEQ ID NO: 150 - protein, 107 aa, synthetic construct; identical to SEQ ID NO: 107
  source 1..107: note = STEAP2 VL 40A3 LO12

SEQ ID NO: 151 - protein, 689 aa, synthetic construct
  source 1..689: note = TED4 LO12 Knob Heavy Chain
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDGGGGSGGGGSEVQLVESGGALVKPGGSLRLSCAASGFTFTEAWMHWVRQAPGKQLEWVAQIKDRSQTYATYYAESVKGRFTISRDDSKNTLYLQMNSLKTEDTAVYYCRGVYYANAPFDYWGQGTLVTVSSASTKGPSVCPLAPSSKSTSGGTADLGCLVKDYFPEPVTVSWNSGALTSGVHTFPAVLQSSGLYSLSSVVTVPSSSLGTQTYICNVNHKPSNTKVDKRVEPKSVDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 214 aa, synthetic construct; identical to SEQ ID NO: 118
  source 1..214: note = TED4 LO12 Kappa Chain

SEQ ID NO: 153 - protein, 595 aa, synthetic construct
  source 1..595: note = TED4 LO12 hole Heavy Chain
EVQLLESGGGLVQPGGSLRLSCAASGFTFDDYAIGWFRQAPGKEREGVSCIRVSDGSTYYADSVKGRFTISRDNSKNTLYLQMNSLRAEDTAVYYCAAGSLYTCVQSIVWPARPYYDMDYWGQGTLVTVSSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKRVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 154 - protein, 218 aa, synthetic construct
  source 1..218: note = TED4 LO12 Lambda Chain
DIVMTQSPLSLPVTPGEPASISCRSSQSLVHNTGNTYLSWYLQKPGQSPQSLIYKVSNRASGVPDRFSGSGSGTDFTLKISRVEAEDVGVYYCGQGTQAPFTFGSGTKVEIKGQPKAAPSVRLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 155 - protein, 13 aa, synthetic construct
  source 1..13: note = IgG1 Hinge
DKTHTCPPCPAPE